SEQUENCE LISTING (4 sequences)

SEQ ID NO: 1 - DNA, 24 bp, synthetic construct
  source 1..24: mol_type = other DNA
gtgtcgcacactgtacccatttat

SEQ ID NO: 2 - DNA, 25 bp, synthetic construct
  source 1..25: mol_type = other DNA
atcttcatcatgtagtcggtcaagt

SEQ ID NO: 3 - DNA, 19 bp, synthetic construct
  source 1..19: mol_type = other DNA
aatcacagcacttgcacca

SEQ ID NO: 4 - DNA, 20 bp, synthetic construct
  source 1..20: mol_type = other DNA
gagggaagcaagaatggaac

(19) United States
(12) Patent Application Publication
(10) Pub. No.: US 2025/0374921 A1
(43) Pub. Date: Dec. 11, 2025

(54) PLANT DEFENSE INDUCER

(71) Applicant: KATHOLIEKE UNIVERSITEIT LEUVEN, Leuven (BE)

(72) Inventors: Bart Lievens, Leuven (BE); Bert Lagrain, Leuven (BE); Bert Sels, Leuven (BE); Bruno Cammue, Leuven (BE); Bruno Pandalone, Leuven (BE); Joëlle Mühlemann, Leuven (BE); Karin Thevissen, Leuven (BE); Korneel van Aelst, Leuven (BE); Ludo Cialdella, Leuven (BE); Thomas Nicolaï, Leuven (BE)

(21) Appl. No.: 19/096,361

(22) Filed: Mar. 31, 2025

(30) Foreign Application Priority Data
Jun. 5, 2024 (EP) 24180126.5
Jul. 1, 2024 (EP) 24185589.9

(51) Int. Cl. A01N 31/16; A01N 25/02; A01N 25/30; A01N 43/08; A01P 1/00; A01P 3/00; A01P 5/00; A01P 7/02; A01P 7/04; A01P 21/00

(52) U.S. Cl. CPC A01N 31/16; A01N 25/02; A01N 25/30; A01N 43/08; A01P 1/00; A01P 3/00; A01P 5/00; A01P 7/02; A01P 7/04; A01P 21/00; C07C 43/23; C07C 43/295

(57) ABSTRACT

Described are plant defense elicitors or plant elicitor compositions and their use in, for example, agricultural applications, more particularly, to protect plants against pests or pathogens. This includes the corresponding methods of and uses in the protection of plants and crops by application of these plant defense elicitors or plant elicitor compositions. The active ingredient(s) of the plant defense elicitor or plant elicitor comprise lignin-derived phenolic oligomers with a degree of polymerization (DP) of 2 to 8 (preferably 2-4) (such as lignin-derived dimeric (diphenolic), lignin-derived trimeric compounds (triphenolic) and lignin-derived tetrameric compounds) and the engineered or synthesized structurally similar compounds. More particularly, described is a plant defense elicitor wherein an active ingredient of the plant defense elicitor comprises depolymerized lignin containing as active ingredient lignin-derived phenolic oligomers (such as lignin-derived dimeric compounds (diphenolic), lignin-derived trimeric compounds (triphenolic), and lignin-derived tetrameric compounds) and engineered or synthesized structurally similar compounds.

### PRIORITY CLAIM

This application claims the benefit of the filing date of European Union Patent Application Serial No. EP24180126, filed Jun. 5, 2024, for “Plant Defense Inducer,” and to European Union Patent Application Serial No. EP24185589, filed Jul. 1, 2024, for “Plant Defense Inducer”, the contents of each of which are incorporated herein by this reference.

### STATEMENT ACCORDING TO 37 C.F.R. § 1.834(c)

Pursuant to 37 C.F.R. § 1.831 through 1.835, a Sequence Listing XML file entitled “3292-P18413US.xml,” 4.23 kilobytes in size, generated Mar. 10, 2025, has been submitted via EFS-Web in lieu of a paper copy. This Sequence Listing is hereby incorporated by reference in its entirety into the specification.

### TECHNICAL FIELD

This application is broadly in the field of agrochemical or phytopharmaceutical compounds and compositions for plant treatment and relates to these compounds or compositions as plant defense elicitors. These are also commonly known as “plant immune system-activator molecules”, or “bio-pesticides” in layman's terms, a class of environmental-friendly molecules that induce or boost plant's resistance against abiotic stressor or biotic stressors such as pests and, e.g., retard the infection and propagation of microbial and viral pathogens on plants and uses thereof. Insects may be a vector of such pathogens.

### BACKGROUND

A need exists for technology to efficiently produce high-quality agricultural products in a limited amount of cultivated land. Therefore, it is of high interest in agriculture to control diseases caused by pests, e.g., parasites or pathogens such as fungi, oomycetes, bacteria, viruses, nematodes and insects. For example, fungicides are chemical compounds or biologic substances used to kill or inhibit fungi or oomycetes or their spores. Fungicides sometimes also have an effect on other plant pathogens such as bacteria, viruses, nematodes or insects. A drawback of using certain fungicides is that fungicide residues can be found, in the environment and on food for human consumption sometimes posing a danger to biodiversity, human or animal health. In general, traditional pesticides, while effective, can harm the environment in several ways. They can pollute soil and water sources, harm beneficial insects and pollinators, and disrupt ecosystems. A need exists in the art to make them unnecessary or to decrease their use.

Thus, in order to promote sustainable crop production, there is a need to use more safe and natural substances with biological activity that can decrease the amounts of pest control chemicals and in particular chemical fungicides needed.

Lignin is the second largest biopolymer on earth. Lignin, with its polyaromatic network is an aromatic polymer is a major constituent in, e.g., wood, being the most abundant carbon source on Earth second only to cellulose. In recent years, with development and commercialization of technologies to extract lignin in a highly purified, solid and particularized form from the pulp-making process, it has attracted significant attention as a possible renewable substitute to primarily aromatic chemical precursors currently sourced from the petrochemical industry.

Native or pristine lignin, a complex polymer consisting of aromatic building blocks, results from a radical polymerization process of three 4-hydroxyphenylpropanoid building blocks or monolignols linking the latter via stable carbon-carbon bonds and more reactive ether bonds. The complex native lignin polymer can be broken down by various catalytic or thermosolvolytic methods of lignin depolymerization of the lignin source, such as reductive catalytic fractionation (RCF), non-catalytic thermosolvolytic depolymerization and fractioning or combinations thereof to cleave the interunit linkages within the lignin polymer (ether bonds) into a liquid (lignin oil) comprising low molecular weight oligomers (oligophenolics having three or more aromatic groups or a degree of polymerization (DP) of three and more), dimers (diphenolics having two aromatic groups or a DP of 2) and monomers (monophenols with one aromatic group, DP of 1). Such lignin oil can be further fractionated, e.g., by ultrafiltration and solubility-based methods, into different fractions of aromatic compounds with a specific DP.

Plant cells have evolved a sophisticated immune system comprising two main layers of defense known as pattern-triggered immunity (PTI) and effector-triggered immunity (ETI) (Song et al. New Phytologist (2022) 236:590-607), that constitute the so-called plant immune system. The plant immune system against insects involves a complex interplay of signaling pathways, defense mechanisms, and elicitors that work together to protect plants from herbivory.

ETI confers a narrow strain-specific resistance as it is initiated following the recognition of virulence effectors (A virulence-proteins) by cytoplasmic resistance genes (“R-genes”). This generally causes a strong site-specific accumulation of reactive oxygen species (ROS) leading to apoptosis. In contrast, PTI provide a broad-spectrum protection. Evolutionarily conserved pathogen-associated molecular-pattern (PAMPs) are sensed by plants through a plethora of plasma membrane-anchored pattern-recognition receptors (PRRs) (Albert et al. Surface Sensor Systems in Plant Immunity, Plant Physiol. 2020, vol. 182(4), 1582-1596).

Compounds, which when perceived by a plant give rise to such defense responses to abiotic and/or biotic stressors, are commonly referred to as plant defense elicitor, plant pest defense elicitor(s), plant immune system elicitor(s), plant defense elicitor(s), plant elicitor(s) or simply elicitor(s). Through the description, the term plant defense elicitor or plant defense elicitors is most used.

The agricultural industry is engaged in a relentless fight against plant pathogens, exacerbated by an ever-changing environment due to inter alia climate changes, striving to avoid major economically early losses and uncertainty in the food supply chain. While only a small variety of chemicals are in common use as pesticides or fungicides, even this reservoir is diminishing, due to emerging biological resistance in plant pathogens, and because of the side effects of some of these chemical compounds on human health. Therefore, there is an urgent need for new plant protection compounds and compositions.

The importance of natural plant defense elicitors lies in their ability to enhance plant defense mechanisms against various pest (pathogens and stressors) such as fungi, oomycetes, bacteria, viruses, nematodes and insects. Natural compounds that induce or boost plant immunity are crucial for activating plant defense responses, thereby inhibiting pathogen development and improving plant resilience against biotic and/or abiotic stress. Compounds, which when perceived by a plant give rise to such defense responses, are commonly referred to as plant defense elicitor, plant pest defense elicitor(s), plant immune system elicitor(s), plant defense elicitor(s), plant elicitor(s) or simply elicitor(s). Through the description the term plant defense elicitor or plant defense elicitors is most used.

### SUMMARY OF THE DISCLOSURE

The disclosure solves this problem by utilizing a novel plant defense elicitor and demonstrates how this can be obtained from a lignin depolymerization process and fractioning in compositions comprising oligophenolics with a degree of polymerization (DP) of 2 to 8 (preferably, 2-4) from depolymerized lignin or decomposed lignin or from structurally identical oligophenolics. They can increase resistance to adverse conditions (biotic or abiotic). The disclosure also demonstrates how these can be used in suitable compositions for plant defense.

The disclosure solves the problems of the related art of plant protection against pathogens and stressors by using more natural compounds or natural sources as a plant defense elicitor, comprising as an active ingredient of the plant defense elicitor depolymerized lignin. Furthermore, this depolymerized lignin comprises specific oligophenolics with a DP from 1 to 8.

Disclosed are new plant defense elicitors or plant elicitor compositions, and their use in agricultural applications, more particularly to protect plants against pests or pathogens or abiotic stressors. This includes the corresponding methods of and uses in the protection of plants and crops by application of these new plant defense elicitors or new plant elicitor compositions. This is characterized in that, an active ingredient(s) of the plant defense elicitor comprises lignin-derived oligophenolics or structural similar compounds with a DP of 2 to 8, and yet more preferably of 2-4 (FIG. 2A-2D). More particularly, the disclosure concerns a plant defense elicitor or plant elicitor described herein, characterized in that, an active ingredient of the plant defense elicitor comprises depolymerized lignin containing as active ingredient lignin-derived oligophenolics with a DP of 2 to 8, (preferably 2-4) or selected structural similar compounds (FIGS. 2A-2D).

Also disclosed are uses of and methods of employing depolymerized lignin or decomposed lignin oligophenolics (FIGS. 1A, 1B) with a DP of 2 to 8 (preferably 2-4) or structural identical oligophenolics as a plant defense elicitor. Also provided are phytopharmaceutical or agrochemical compositions comprising depolymerized lignin or decomposed lignin oligophenolics with a DP of 2 to 8 (preferably 2-4) or structural identical oligophenolics, and applications thereof. In certain preferred embodiments, the compositions may further comprise other plant elicitors or may comprise antifungal, antimicrobials or antiviral compounds. In certain preferred embodiments, the compositions may be produced by decomposition of lignin by reductive catalytic fractionation (“RCF”) (FIG. 1A), non-catalytic thermosolvolytic de-polymerization (FIG. 1A) and fractioning (FIG. 1C) of the lignin source.

An aspect of the disclosure concerns a plant elicitor (elicitor of natural plant defenses) characterized in that an active ingredient of the plant defense elicitor comprises phenolics derived from depolymerized lignin, including lignin-derived dimeric (diphenolic), lignin-derived trimeric compounds (triphenolic) and lignin derived tetrameric (tetraphenolic) compounds and engineered or synthesized structural similar compounds. More particularly, the disclosure concerns a plant defense elicitor described herein, wherein an active ingredient of the plant defense elicitor comprises depolymerized lignin containing as active ingredient lignin-derived diphenolics.

Disclosed is a method for controlling a plant disease comprising treating a plant with a plant defense elicitor or elicitor containing phenolics derived from depolymerized lignin of the RCF or the non-catalytic thermosolvolytic depolymerization process. This plant defense elicitor can be a lignin oil and fractions thereof (with an approach exemplified in FIG. 1C) applied, e.g., at a concentration of 0.05 to 20 mg/ml, preferably 0.2 to 10 mg/ml, more preferably 0.5 to 5 mg/ml, even more preferably 0.8 to 1.2 mg/ml and most preferably 1 mg/mL and, e.g., comprising molecular mass (weight average mass or Mw) of 180 g/mol to 1800 g/mol, preferably between 230 g/mol to 1000 g/mol, and yet more preferably between 230 g/mol to 650 g/mol of active ingredient compounds (FIGS. 2A-2D) or in case of a dry composition 0.5 to 30 wt % by dry weight of aromatic compounds, preferably 1 to 20 wt % by dry weight of aromatic compounds, more preferably 2 to 10 wt % by dry weight of aromatic compounds, wherein the aromatic compound comprise at least one aromatic compound from the following formulae:
I) at least one aromatic compound selected from the formulae

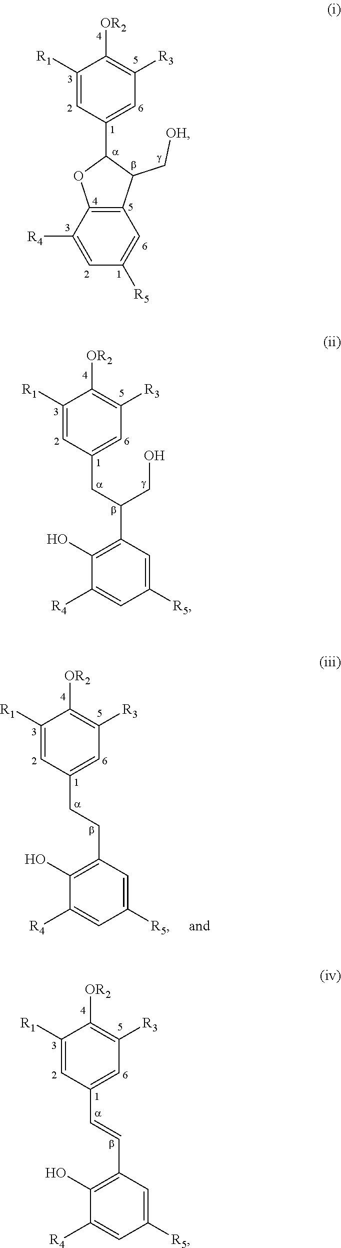

and
wherein each R₁, R₃, and R₄ is independently chosen from —H, —OH, O—CH3, a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or aromatic oligomer, or a carbon-oxygen linkage to an aromatic monomer or aromatic oligomer,
wherein R₂ is —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer, or a carbon-oxygen linkage to an aromatic monomer or aromatic oligomer,
wherein R₅ is selected from —H, a β-O-4 linkage to an aromatic monomer or aromatic oligomer, a β-5 linkage to an aromatic monomer or aromatic oligomer, a β-β linkage to an aromatic monomer or aromatic oligomer, a β-1 linkage to an aromatic monomer or aromatic oligomer, an end-unit selected from CH₃, —CH₂CH₃, —(CH₂)₂CH₃, —CH₂CH═CH₂, —CH═CHCH₃, —(CH₂)₂CH₂OH, —(CH₂)₂CHO, —CH═CHCH₂OH, —(CH₂)₂CH₂OCH₃, —CH═CHCH₂OCH₃, —(CH₂)₂CH₂OCH₂CH₃, —CH═CHCH₂OCH₂CH₃, —(CH₂)₂CH₂O(CH₂)₂CH₃, —CH═CHCH₂O(CH₂)₂CH₃, —(CH₂)₂CH₂OCH(CH₃)₂, —CH═CHCH₂OCH(CH₃)₂, —(CH₂)₂CH₂O(CH₂)₃CH₃, —CH═CHCH₂O(CH₂)₃CH₃, a carbon linkage to an aromatic monomer or aromatic oligomer;
II) at least one aromatic compound selected from the formulae

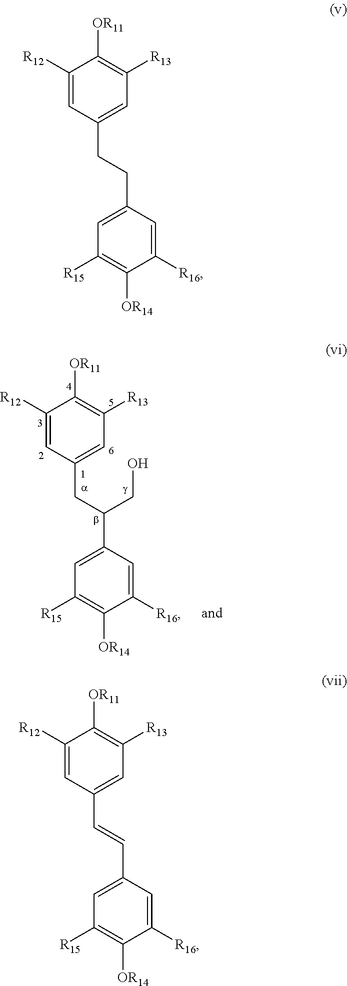

and
wherein each R₁₂, R₁₃, R₁₅ and R₁₆ is independently chosen from —H, —OH, O—CH₃, a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or aromatic oligomer, or a carbon-oxygen linkage to an aromatic monomer or aromatic oligomer,
wherein R₁₁ and R₁₄ is independently chosen from —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer or a carbon-oxygen linkage to an aromatic monomer or aromatic oligomer, and/or
III) wherein at the aromatic compounds comprise at least one aromatic compound selected from the formula (viii)

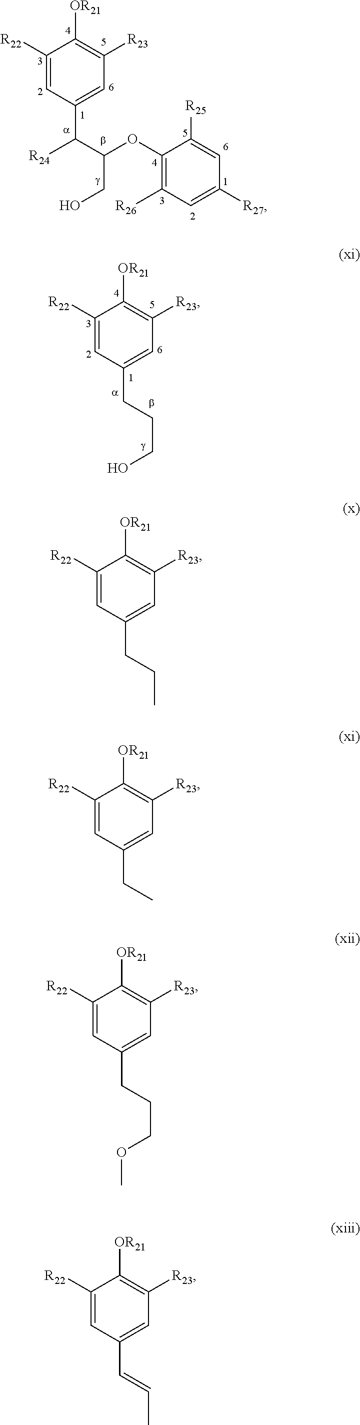

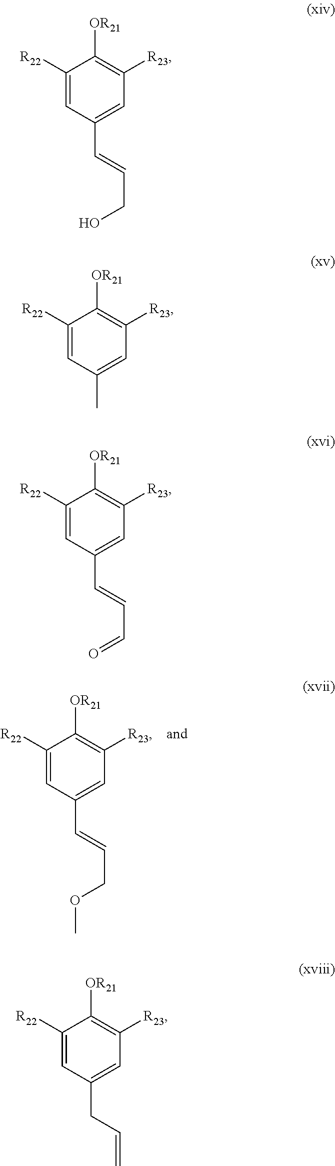

each with at least one linkage to an aromatic monomer or aromatic oligomer and
wherein each R₂₂, R₂₃, R₂₅ and R₂₆ is independently chosen from —H, —OH, O—CH₃, a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or an aromatic oligomer, or a 4 carbon-oxygen linkage to an aromatic monomer or aromatic oligomer,
wherein R₂₁ is independently chosen from —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer or a carbon-oxygen linkage to an aromatic monomer or aromatic oligomer,
wherein R₂₄ is independently chosen from —H, OH, or —O-Alkyl wherein the alkyl group is derived from the alcohol solvent of the process, wherein R₂₇ is independently chosen from —H, a β-O-4 linkage to an aromatic monomer or aromatic oligomer, a β-5 linkage to an aromatic monomer or aromatic oligomer, a β-β linkage to an aromatic monomer or aromatic oligomer, a β-1 linkage to an aromatic monomer or aromatic oligomer, end-unit selected from CH₃, —CH₂CH₃, —(CH₂)₂CH₃, —CH₂CH═CH₂, —CH═CHCH₃, —(CH₂)₂CH₂OH, —(CH₂)₂CHO, —CH═CHCH₂OH, —(CH₂)₂CH₂OCH₃, —CH═CHCH₂OCH₃, —(CH₂)₂CH₂OCH₂CH₃, —CH═CHCH₂OCH₂CH₃, —(CH₂)₂CH₂O(CH₂)₂CH₃, —CH═CHCH₂O(CH₂)₂CH₃, —(CH₂)₂CH₂OCH(CH₃)₂, —CH═CHCH₂OCH(CH₃)₂, —(CH₂)₂CH₂O(CH₂)₃CH₃, —CH═CHCH₂O(CH₂)₃CH₃, a carbon linkage to an aromatic monomer or an aromatic oligomer.

Further disclosed is an engineered composition comprising aromatic compounds, wherein the molecular mass (weight average or Mw) of the aromatic compounds is between 180 g/mol to 1000 g/mol (preferably between 230 g/mol to 650 g/mol), or wherein the aromatic compounds comprise, consist essentially of, or consist of lignin derived phenolics with a DP of 2 to 8, preferably 2-4 or synthesized structurally similar compounds. These compounds were obtained the RCF (FIG. 1A) or the non-catalytic thermosolvolytic depolymerization (FIG. 1B) process of lignin, lignocellulose or biomass comprising lignocellulose or lignin.

In one aspect, these defined phenolic structures are from lignin depolymerization.

Further scope of applicability of the disclosure will become apparent from the detailed description given herein. However, it should be understood that the detailed description and specific examples, while indicating preferred embodiments of the disclosure, are given by way of illustration only, since various changes and modifications within the spirit and scope of the disclosure will become apparent to those skilled in the art from this detailed description. It is to be understood that both the foregoing general description and the following detailed description are exemplary and explanatory only and are not restrictive of the disclosure, as claimed.

The compounds and combinations of or composition comprising the compounds as taught herein are put to use as plant defense elicitors. This term broadly encompasses any compounds or compositions that are capable of eliciting natural plant defenses, of activating plant defense and resistance reactions against plant pathogen, of stimulating the production of plant defense molecules against plant pathogen and/or of preventing, controlling or treating a plant against infection by a plant pathogen and/or against abiotic stressors, when administered to a plant, plant seed or an organ of a plant. Hence, when perceived by a plant, an elicitor can evoke molecular, biochemical and/or physiological defensive plant cell reactions, such as the synthesis, or increase of the synthesis, of plant defense molecule(s), for example, ethylene and/or salicylic and jasmonic acid, the production of reactive oxygen species (ROS), and/or expression of specific defense-related genes and proteins, for example, polygalacturonase inhibitor proteins (PGIP). The activation of signal transduction pathway(s) can subsequently lead to long-lasting defense gene expression and secondary metabolites production. As a result, a plant defense elicitor can enhance the ability of the plant to resist or battle a given pathogen. Plant defense elicitors are often subsumed by the group of agents colloquially known as biopesticides, which mainly include bioinsecticides, biofungicides, bionematicides, and others.

Hence, in certain embodiments, by means of the present compounds and/or compositions, a plant infection by a plant pathogen may be prevented, controlled or treated. While these terms are well-known as such, by means of further guidance, “preventing” may in particular mean avoiding occurrence of at least one adverse effect or symptom, preferably all adverse effects or symptoms induced by a plant pathogen infection; “controlling” may in particular mean stopping the progression of a plant pathogen infection, more precisely reducing or abolishing the plant pathogen spread across the healthy parts of a plant or of an organ of a plant, or from an infected plant to another plant, typically to a neighboring plant; and “treating” may in particular mean ameliorating the symptom(s) of an infection, or completely curing an infection, typically by reducing or completely eliminating a phytopathogen (typically a fungus or bacterium), i.e., by eliminating any viable phytopathogen in the plant or in an organ or several or each organ(s) of the plant.

A plant may be contacted with an effective amount of the present compounds or compositions, such as contacted via an organ of the plant, preferably an organ selected from leaves, roots and/or fruits, or via seeds of the plant. The contacting step may be performed once or several times (e.g., regularly or periodically, for example, on the appropriate season or at the appropriate plant development stage). The term “an effective amount” refers to an amount of the (active) compound or compounds as taught herein which induces or elicits plant natural defense, activate plant defense and resistance reaction against abiotic stressors or against plant pathogen, and/or stimulates the production of plant defenses molecules against plant pathogen resulting in obtaining a plant that is resistant to pathogen(s). The effective amount is understood to be variable, as it may be affected by many factors, including but not limited to the type of plant treated, treatment dosages and application rates, method of contacting, weather and seasonal conditions experienced during the plant growing cycle, pathogen susceptibility, etc. Such variables are commonly encountered and understood by the skilled person, who may adjust the prophylactic or treatment regimen, e.g., application rate, application timings and/or frequencies, and application way. Particularly suitable amounts or concentrations ranges are discussed and exemplified elsewhere in this specification. The terms “organ,” “organ of a plant” or “plant's organ” interchangeably refer to a part of a plant or to a plant propagation material. Examples of plant's organs include, but are not limited to, leaves, stems, fruits, seeds, cuttings, tubers, roots, bulbs, rhizomes, and the like. The contacting step with the plant or organ can be performed in various ways, for example, by spraying, drenching, soaking, dipping, injection, through soil feeding, and any combination thereof. Alternatively, the compounds or compositions can be applied on a plant or organ by supplying a volatile or vapor-based form of the compounds or compositions in the vicinity of the plant tissue and allowing the diffusion to the plant or organ through the atmosphere. The skilled person knows how to adapt the manner of administration to the particular use.

In the context of the disclosure, the term “plant” typically designates a plant infected by or presenting a susceptibility to infection by a plant pathogen or affected by an abiotic stressor. For example, the plant may belong to the clade of Angiosperm.

In certain embodiments, the plant may belong to the clade of dicots. Examples of plants from the dicots clade include, but are not limited to, the Solcmcicecie family, comprising Solcinum lycopersicum (tomato), Solcinum tuberosum (potatoes), Solcinum melongenci (eggplant), Capsicum genus (pepper) and Nicotiana tabacum (tobacco); the Vitaceae family comprising the Vitis genus (grapevines); the Brassicaceae family, comprising Brassica oleracea (cabbage), Brassica rapa (turnip and Chinese cabbage), mustard species and A. thaliana; the Rosaceae family, comprising Malus pumila (apple), Pyrus species (pear), and Fragaria ananassa (strawberry); the Fabaceae family comprising legumes such as pea, bean and soybean; the Asteraceae family, comprising sunflower; Amaranthaceae family, comprising sugar beet.

In certain embodiments, the plant may belong to the clade of monocots. An example of plants from the monocot clade includes, but is not limited to, the Gramineae or Poaceae family, such as maize, rice, barley, or wheat.

Hence, in certain embodiments, the plant may be a dicot plant, preferably selected from Brassicaceae, Solanaceae or Rosacea families, such as A. thaliana, cabbage, tomato, grapevine, soybean, apple, pear, or strawberries.

In certain embodiments, the plant pathogen may be a fungus or a bacterium a hemibiotrophic fungus or bacterium, or a biotrophic fungus. Hence, in such case the compositions as taught herein may also be conveniently denoted as antifungal and/or an antibacterial adjuvant, i.e., products that assist in the prevention or treatment of a plant disease typically caused by fungi or bacteria.

During the colonization of plant hosts, most fungal pathogens exhibit one of two modes of nutrition: biotrophy, in which nutrients are obtained from living host cells, and necrotrophy, in which nutrients are obtained from host cells which have been previously killed by the fungus. A third mode of nutrition is hemibiotrophy, where the pathogen has an initial period of biotrophy followed by a period of necrotrophy. Phytopathogenic pathogens, in particular fungi, can thus be distinguished depending on their mode of nutrition: necrotrophic (e.g., Botrytis cinerea), biotrophic (e.g., Ustilago maydis) or hemibiotrophic (e.g., Colletotrichum higginsianum). In particular embodiments, the plant pathogen may be a fungus, typically a phytopathogenic fungus. The expression “phytopathogen fungus” refers to fungi pathogens that infect plant organs. Examples of phytopathogenic fungi include, but are not limited to, fungi belonging to the Ascomycetes and Basidiomycetes classes, such as, for example, fungi of the order of Helotiales, such as, for example, family Sclerotiniaceae, Botrytis/Botryotinia, such as species Botrytis cinerea, fungi of the order of Hypocreales, such as, for example, family Nectriaceae, genus Fusarium, fungi of the order of Uredinales, such as, for example, family Pucciniaceae, genus Puccinia, fungi of the order of Ustilaginales, such as, for example, family Ustilaginaceae, genus Ustilago), fungi of the order of Sordariomycetes, such as, for example, family Glomerellaceae, genus Colletotrichum.

In further embodiments, the plant pathogen may be a bacterium, typically a phytopathogenic bacterium. The expression “phytopathogen bacterium” refers to bacterial pathogens that infect plant organs. Examples of phytopathogenic bacteria include, but are not limited to, bacteria of the order of Pseudomonadales, such as, for example, family Pseudomonadaceae, genus Pseudomonas, such as species Pseudomonas syringae, bacteria of the order of Burkholderiales, such as, for example, family Burkholderiaceae, genus Ralstonia, such as species Ralstonia solanacearum, bacteria of the order of Enterobacterales, such as, for example, family Erwiniaceae, genus Erwinia, such as species Erwinia amylovora, or family Pectobacteriaceae, genus.

Pectobacterium, such as species Pectobacterium carotovorum (formerly Erwinia carotovora, bacteria of the order of Xanthomonadales, such as, for example, family Xanthomonadaceae, genus Xylella, such as species Xylella fastidiosa, or genus Xanthomonas, such as species Xanthomonas campestris. Similarly to the fungi and based on the type of plant colonization, bacteria can also be classified into necrotrophic, biotrophic and hemibiotrophic sub-classes.

Particularly preferred may be necrotrophic fungi, preferably as Botrytis cinerea. Hence, in certain embodiments, the plant pathogen is a fungus or a bacterium, such as a necrotrophic fungus or bacterium, such as Botrytis cinerea, a hemibiotrophic fungus or bacterium, or a biotrophic fungus or bacterium, such as Pseudomonas syringae.

In the present context, the plant infection by a plant pathogen typically designates a plant infection by at least one phytopathogen. The infection can occur on any organ of the plant. By means of examples and without limitation, the plant infection may be, e.g., a B. cinerea infection, for example, a B. cinerea infection of A. thaliana, tomato, strawberry, sunflower, grapevine, or apple; a C. higginsianum infection, for example, a C. higginsianum infection of turnip, Chinese cabbage, mustard, A. thaliana, or apple; a U. maydis infection, for example, a U. maydis infection of maize; a R. solanacearum infection, for example, a R. solanacearum infection of tomato, potatoes, eggplant, pepper, or tobacco; a Pseudomonas syringae infection, for example, a Pseudomonas syringae infection on apple or pear; or any combination thereof, such as a B. cinerea and/or C. higginsianum infection of A. thaliana, an apple infection by B. cinerea and/or C. higginsianum or a tomato infection by B. cinerea and/or R. solanacearum.

In certain embodiments, the concentration of one or more oligophenolics with a DP of 2 to 8 (preferably 2-4) (FIGS. 2A-2D) from depolymerized lignin or decomposed lignin or from structural identical oligophenolics (e.g., the structures and the structures in a (phytopharmaceutical or agrochemical) composition as taught herein) is 180 g/mol to 1000 g/mol, and yet more preferably between 230 g/mol to 650 g/mol.

Some of the methods described herein may be embodied as that the lignin-derived aromatic oligomers are phenolics comprising two benzene rings directly bridged or bridged with a common bridging group of the group consisting of aliphatic chains, alkene groups, carbonyl groups and ether linkages or wherein the phenolic oligomers have two aromatic groups.

Yet some of the methods described herein may be embodied as that the lignin-derived aromatic oligomers are phenolics comprising two benzene rings directly bridged or bridged with a common bridging group of the group consisting of —CH₂— groups, —CH═CH—, —C(═O)-and —O—.

Preferably these lignin-derived aromatic oligomers are substantially free of acetic acid, methanol and ethanol, meaning it contains less than 0.1% of each acetic acid, methanol and ethanol. Furthermore, preferably these lignin-derived aromatic oligomers are comprised in a composition with a pH in the range of 4 to 10, preferably in the range of 5 to 8 or in origin have a pH in the range of 4.0 to 6.0.

In another aspect, the disclosure provides that the lignin-derived aromatic oligomers are comprised in a composition, further comprising an ingredient of the group consisting of a surfactant, a biosurfactant, a penetration enhancer, a dispersing agent, an emulsifier and a carrier or a combination thereof.

In another aspect, the disclosure provides that these lignin-derived aromatic oligomers are comprised in a composition, further comprising a repolymerization inhibitor of the group of a carbocation scavenger, aromatic scavengers and a polyhydric alcohol or a combination thereof or a repolymerization inhibitor of the group consisting of citric acid, salicylic acid, 2-naphthol, phenolic acids (e.g., vanillic acid, syringic acid), ethylene glycol, glycerol, mannitol (C₆H1₄O₆), sorbitol (C₆H₁₄O₆), xylitol (C₅H₁₂O₅), erythritol, maltitol (C₁₂H₂₄O₁₁) or a combination thereof.

In another aspect, the disclosure provides that these lignin-derived aromatic oligomers are 0.5 to 30 wt % by dry weight of aromatic compounds, preferably 1 to 20 wt % by dry weight of aromatic compounds, more preferably 2 to 10 wt % of the composition in dry state.

Some of the methods described herein may be embodied as promoting induced systemic resistance, for inducing latent host defenses, or for priming the intrinsic resistance mechanisms in a plant, comprising applying to a plant or a plant part, the composition described in these methods above.

Some of the methods described herein may be embodied as promoting induced systemic resistance, for inducing latent host defenses or for priming the intrinsic resistance mechanisms in a plant, comprising by spraying on the plant or contacting the roots of the plant with the lignin-derived aromatic oligomers or a composition therewith.

Some of the methods described herein may be embodied as the methods of the disclosure for protecting plants against plant pests, comprising applying an effective and substantially non-phytotoxic amount of the lignin-derived aromatic oligomers to the plants, e.g., wherein the plant pests are selected from the group comprising: fungi, oomycetes, bacteria, viruses, nematodes and insects.

Some of the methods described herein may be embodied as the method according to any of statements thereon hereinabove described, wherein the composition is applied before harvest or post-harvest to the whole plant, the leaves, the flowers, fruits, seeds, seedlings or seedlings pricking out, propagation material such as tubers or rhizomes, plants pricking out, and/or to the soil or inert substrate wherein the plant is growing or in which it is desired to grow, by spraying, drenching, soaking, dipping, injection or administration through fertilizing or irrigation systems.

These inventive methods of plant treatment comprising applying to a plant or a plant part, a composition comprising the plant defense elicitor of the disclosure are suitable for promoting induced systemic resistance in a plant, for inducing latent host defenses of a plant or for priming the intrinsic resistance mechanisms in the plant.

By using these inventive methods of plant treatment it is possible activating plant defense against a pathogen stressor and/or abiotic stressor in a plant selected from the group comprising: cotton, flax, vine, fruit, vegetable, major horticultural and forest crops such as: rosaceae sp., ribesioidae sp., juglandaceae sp., betulaceae sp., anacardiaceae sp., fagaceae sp., moraceae sp., oleaceae sp., actinidaceae sp., lauraceae sp., musaceae sp., rubiaceae sp., theaceae sp., sterculiceae sp., rutaceae sp., solanaceae sp., vitaceae sp., liliaceae sp., asteraceae sp., umbelliferae sp., cruciferae sp., chenopodiaceae sp., cucurbitaceae sp., papilionaceae sp., such as graminae sp., fabacae sp.

These methods of plant treatment hereof by spraying on the plant or contacting the roots of the plant with the plant defense elicitor of any one of above stated methods are suitable for promoting induced systemic resistance of a plant, for inducing latent host defenses of a plant or for priming the intrinsic resistance mechanisms in a plant.

A further embodiment of the disclosure concerns use of lignin-derived aromatic oligomers of the disclosure and described hereinabove as a plant defense elicitor. With this use, the induced systemic resistance can be promoted in a plant or the latent host defenses in a plant can be induced by priming of the intrinsic disease resistance mechanisms of a plant.

Also disclosed is the use of the plant defense elicitor of the disclosure on a plant or parts thereof wherein the latent host defenses are activated preventive to or in the event of an attack by a phytopathogenic pathogen or pest.

Also disclosed is the use of the plant defense elicitor of the disclosure on a plant or parts thereof wherein the latent host defenses are activated preventive to or in the event of an attack by a phytopathogenic pathogen or pest wherein the phytopathogenic pathogen or pest is selected from the group of a fungi, a bacteria, and an insect.

Also disclosed is the use of the plant defense elicitor of the disclosure on a plant or parts thereof wherein the latent host defenses are activated preventive to or in the event of an attack by a phytopathogenic of the group consisting of fungi, bacteria, viruses, viroids, mycoplasma-like organisms, protozoa, insects, acari, and nematodes.

Also disclosed is the use of the plant defense elicitor of the disclosure on a plant or parts thereof wherein the latent host defenses are activated preventive to or in the event of abiotic stress.

Also disclosed is the use of the plant defense elicitor of the disclosure on a plant or parts thereof to prime the intrinsic resistance mechanisms of a plant for stronger or faster induced plant defense preventive to or in the event of an attack by a phytopathogenic pathogen or pest or of abiotic stress, as compared to control or other plant defense inducers. Hereby, the plant defense elicitor can be used in foliar spray agents or in a root drench.

Also disclosed is the use of the plant defense elicitor of the disclosure in agricultural applications or to protect plants against plant pests. Such plant pests can be selected from the group comprising: phytopestic fungi, oomycetes, bacteria, viruses, nematodes and insects.

The disclosure further concerns the use of lignin-derived aromatic oligomers of the disclosure as plant defense elicitors to enhance the efficacy of the fungicide in the composition, or to stimulate the plant immune system. Such use thereof can be on a plant or parts thereof wherein the latent host defenses are activated preventive to or in the event of an attack by a phytopathogenic pathogen or pest or by an abiotic stressor, or such use can be on a plant or parts thereof wherein the latent host defenses are activated preventive to or in the event of an attack by a phytopathogenic pathogen or pest wherein the phytopathogenic pathogen or pest is selected from the group of a fungi, a bacteria, and an insect. It can be on a plant or parts thereof wherein the latent host defenses are activated preventive to or in the event of an attack by a phytopathogenic of the group consisting of fungi, bacteria, viruses, viroids, mycoplasma-like organisms, protozoa, insects, acari, and nematodes, on a plant or parts thereof wherein the latent host defenses are activated preventive to or in the event of abiotic stress or on a plant or parts thereof to prime the intrinsic resistance mechanisms of a plant for stronger or faster induced plant defense preventive to or in the event of an attack by a phytopathogenic pathogen or pest or of abiotic stress, as compared to control or other plant defense inducers.

A further embodiment is a method for producing phytopharmaceutical or agrochemical compositions of the disclosure, the method comprising a) subjecting lignin or lignocellulose in a liquid phase to solvolytic lignin depolymerization, b) fractionating the extract to obtain a purified fraction enriched in lignin oligomers, and c) formulating the purified fraction into a phytopharmaceutically or agrochemically acceptable dosage form.

Yet a further embodiment is a method for producing phytopharmaceutical or agrochemical compositions of the disclosure, wherein the liquid phase described here above contains one or more solvents selected from the group comprising water, methanol, ethanol, n-propanol and isopropyl alcohol and mixtures of two or more thereof.

In yet a further embodiment, the method for producing a phytopharmaceutical or agrochemical composition of the disclosure, wherein the solvolytic lignin depolymerization and fractioning is of the group consisting of reductive catalytic fractionation (RCF), non-catalytic thermosolvolytic depolymerization and oxidative catalytic fractioning (OCF).

According to the disclosure, there is provided a phytopharmaceutical or agrochemical composition, wherein an effective dose of plant defense elicitor compounds that are lignin-derived oligomer aromatics with a DP of 2 to 8 (preferably 2-4) or synthesized structurally similar compounds.

A further embodiment of the disclosure also provides that the plant defense elicitor compounds are from a lignin that is depolymerized or decomposed by reductive catalytic fractionation (RCF) of lignin or lignocellulose or that the plant defense elicitor compounds are from a lignin that is depolymerized or decomposed by non-catalytic thermosolvolytic depolymerization of lignin or lignocellulose. In another aspect, the disclosure provides that the plant defense elicitor compounds are from a lignin that is depolymerized or decomposed or 1) by a reductive catalytic fractionation (RCF) (FIG. 1A) of the lignin source with a heterogenous metal catalyst, including but not limited to Ru, Pd and Ni, on a support in an organic solvent or an organic solvent water mixture in a temperature range of 100° C. to 300° C., preferably 150° C. to 270° C. and most preferably 200° C. to 250° C. and containing a hydrogen donor, including but not limited to H2 or 2) by a non-catalytic thermosolvolytic de-polymerization (FIG. 1B) of the lignin source in an organic solvent or an organic solvent water mixture in a temperature range of 100° C. to 300° C., preferably 150° C. to 270° C. and most preferably 200° C. to 250° C. and under an inert atmosphere. These plant defense elicitor compounds can thus be the reaction product of these processes of lignin depolymerization. This embodiment of the disclosure advantageously comprises that the plant defense elicitor compounds are obtained from lignin depolymerization or decomposing by reductive catalytic fractionation (RCF), as this resulted to the most stable compositions when no re-polymerization where add.

In another aspect, the disclosure provides that the phytopharmaceutical or agrochemical composition has a pH in the range of 4 to 10, preferably in the range of 5 to 8 or that the plant defense elicitor compounds in origin have a pH in the range of 4.0 to 6.0. Some of the compositions described above may be embodied as substantially free of acetic acid, methanol and ethanol, meaning it contains less than 0.1% of each acetic acid, methanol and ethanol.

In a further embodiment, the compositions described above is characterized in that the plant defense elicitor compounds comprise, consist essentially of, or consist of I) at least one aromatic compound selected from the formulae

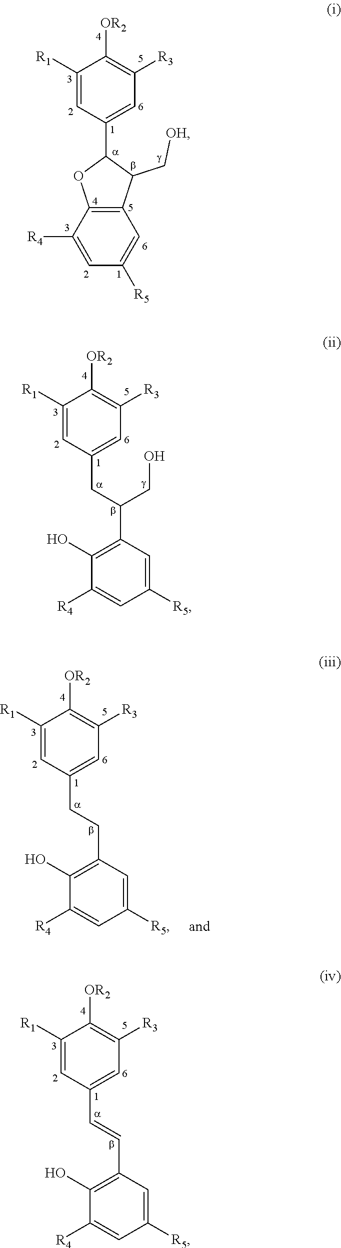

and
wherein each R₁, R₃, and R₄ is independently chosen from —H, —OH, O—CH₃, a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or aromatic oligomer, or a carbon-oxygen linkage to an aromatic monomer or aromatic oligomer,
wherein R₂ is —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer, or a carbon-oxygen linkage to an aromatic monomer or aromatic oligomer,
wherein R₅ is selected from —H, a β-O-4 linkage to an aromatic monomer or aromatic oligomer, a β-5 linkage to an aromatic monomer or aromatic oligomer, a β-β linkage to an aromatic monomer or aromatic oligomer, a β-1 linkage to an aromatic monomer or aromatic oligomer, an end-unit selected from CH₃, —CH₂CH₃, —(CH₂)₂CH₃, —CH₂CH═CH₂, —CH═CHCH₃, —(CH₂)₂CH₂OH, —(CH₂)₂CHO, —CH═CHCH₂OH, —(CH₂)₂CH₂OCH₃, —CH═CHCH₂OCH₃, —(CH₂)₂CH₂OCH₂CH₃, —CH═CHCH₂OCH₂CH₃, —(CH₂)₂CH₂O(CH₂)₂CH₃, —CH═CHCH₂O(CH₂)₂CH₃, —(CH₂)₂CH₂OCH(CH₃)₂, —CH═CHCH₂OCH(CH₃)₂, —(CH₂)₂CH₂O(CH₂)₃CH₃, —CH═CHCH₂O(CH₂)₃CH₃, a carbon linkage to an aromatic monomer or aromatic oligomer;
II) wherein the aromatic compounds comprise at least one aromatic compound selected from the formulae (v)

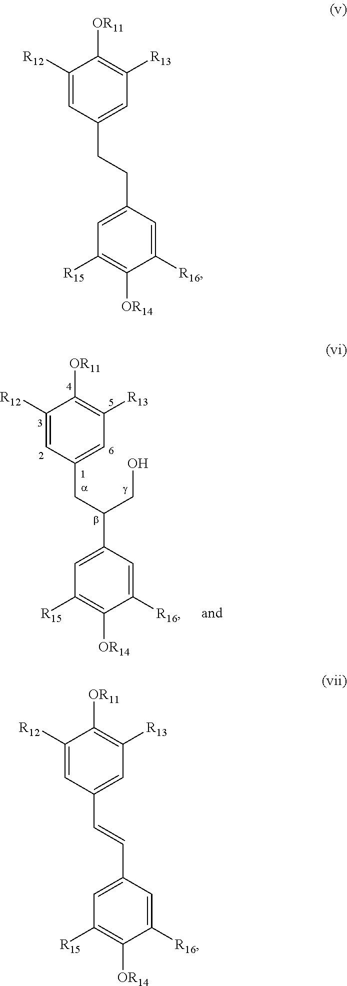

and
wherein each R₁₂, R₁₃, R₁₅ and R₁₆ is independently chosen from —H, —OH, O—CH₃, a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or aromatic oligomer, or a carbon-oxygen linkage to an aromatic monomer or aromatic oligomer,
wherein R₁₁ and R₁₄ is independently chosen from —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer or a carbon-oxygen linkage to an aromatic monomer or aromatic oligomer, and/or
III) wherein at the aromatic compounds comprise at least one aromatic compound selected from the formula (viii)

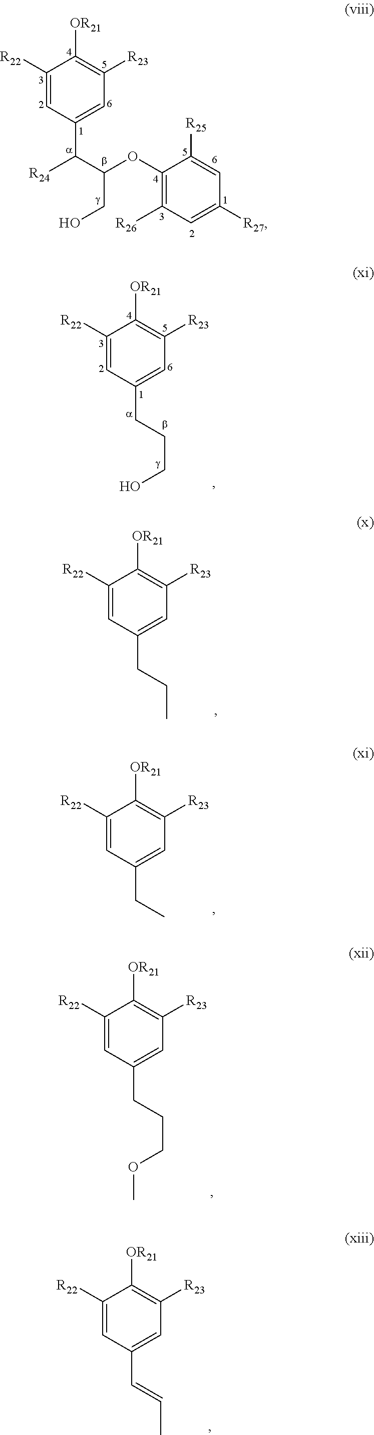

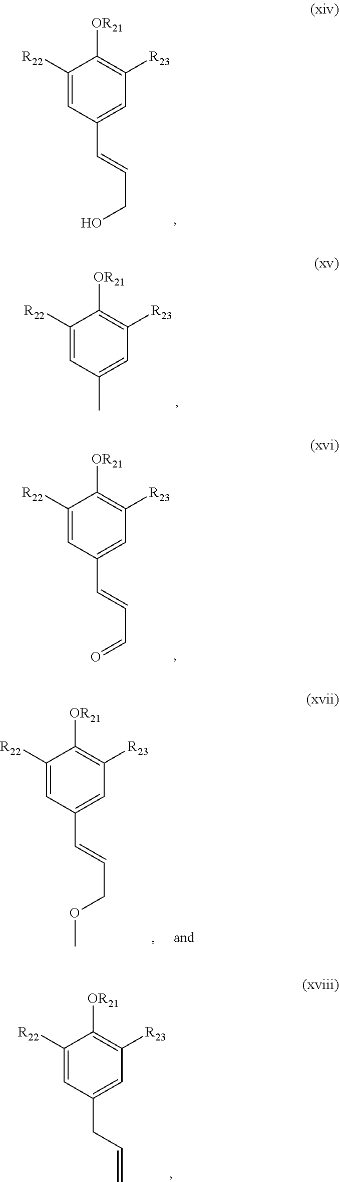

each with at least one linkage to an aromatic monomer or aromatic oligomer and
wherein R₂₁ is independently chosen from —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer or a carbon-oxygen linkage to an aromatic monomer or aromatic oligomer,
wherein each R₂₂ and R₂₃ is dependently chosen from —H, —OH, O—CH₃ or from a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or an aromatic oligomer, or a carbon-oxygen linkage to an aromatic monomer or aromatic oligomer,
wherein R₂₄ is independently chosen from —H, OH, or —O-Alkyl wherein the alkyl group is derived from the alcohol solvent of the process,
wherein each R₂₅ and R₂₆ is independently chosen from —H, —OH, O—CH₃, a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or an aromatic oligomer, or a carbon-oxygen linkage to an aromatic monomer or aromatic oligomer,
wherein R₂₇ is independently chosen from —H, a β-O-4 linkage to an aromatic monomer or aromatic oligomer, a β-5 linkage to an aromatic monomer or aromatic oligomer, a β-β linkage to an aromatic monomer or aromatic oligomer, a β-1 linkage to an aromatic monomer or aromatic oligomer, end-unit selected from CH₃, —CH₂CH₃, —(CH₂)₂CH₃, —CH₂CH═CH₂, —CH═CHCH₃, —(CH₂)₂CH₂OH, —(CH₂)₂CHO, —CH═CHCH₂OH, —(CH₂)₂CH₂OCH₃, —CH═CHCH₂OCH₃, —(CH₂)₂CH₂OCH₂CH₃, —CH═CHCH₂OCH₂CH₃, —(CH₂)₂CH₂O(CH₂)₂CH₃, —CH═CHCH₂O(CH₂)₂CH₃, —(CH₂)₂CH₂OCH(CH₃)₂, —CH═CHCH₂OCH(CH₃)₂, —(CH₂)₂CH₂O(CH₂)₃CH₃, —CH═CHCH₂O(CH₂)₃CH₃, a carbon linkage to an aromatic monomer or an aromatic oligomer.

In a further embodiment, the compositions described above is characterized in that the plant defense elicitor compounds comprise, or essentially consist of or consist of I) at least one aromatic compound selected from the formulae

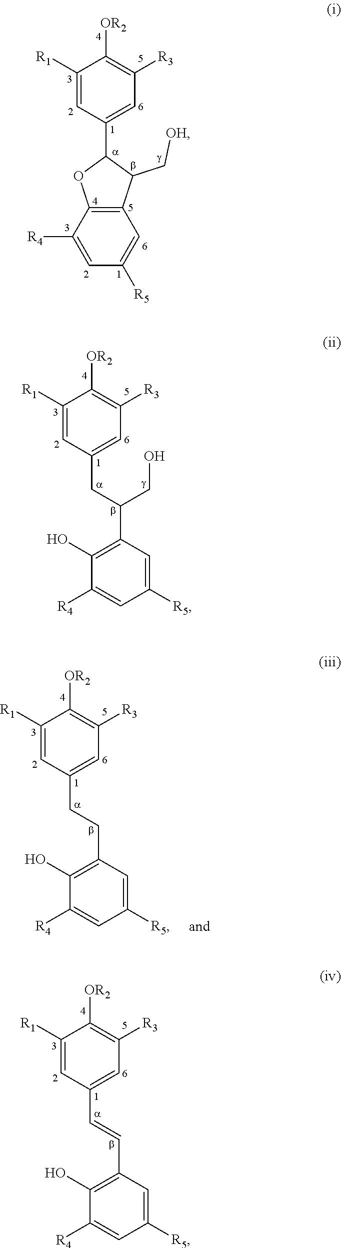

and
wherein each R₁, R₃, and R₄ is independently chosen from —H, —OH, O—CH₃,
wherein R₂ is —H,
wherein R₅ is selected from —H, an end-unit selected from CH₃, —CH₂CH₃, —(CH₂)₂CH₃, —CH₂CH═CH₂, —CH═CHCH₃, —(CH₂)₂CH₂OH, —(CH₂)₂CHO, —CH═CHCH₂OH, —(CH₂)₂CH₂OCH₃, —CH═CHCH₂OCH₃, —(CH₂)₂CH₂OCH₂CH₃, —CH═CHCH₂OCH₂CH₃, —(CH₂)₂CH₂O(CH₂)₂CH₃, —CH═CHCH₂O(CH₂)₂CH₃, —(CH₂)₂CH₂OCH(CH₃)₂, —CH═CHCH₂OCH(CH₃)₂, —(CH₂)₂CH₂O(CH₂)₃CH₃, —CH═CHCH₂O(CH₂)₃CH₃,
II) wherein the aromatic compounds comprise at least one aromatic compound selected from the formulae

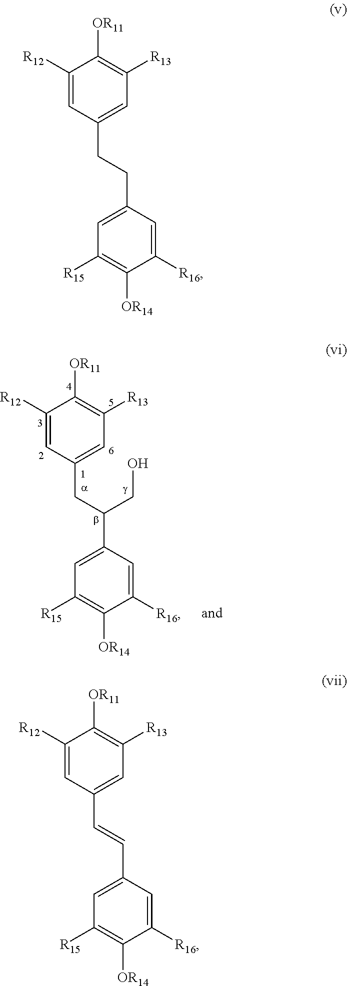

and
wherein each R₁₂, R₁₃, R₁₅ and R₁₆ is independently chosen from —H, —OH, O—CH3,
wherein R₁₁ and R₁₄ is independently chosen from —H, and/or
III) wherein at the aromatic compounds comprise at least one aromatic compound selected from the formula (viii)

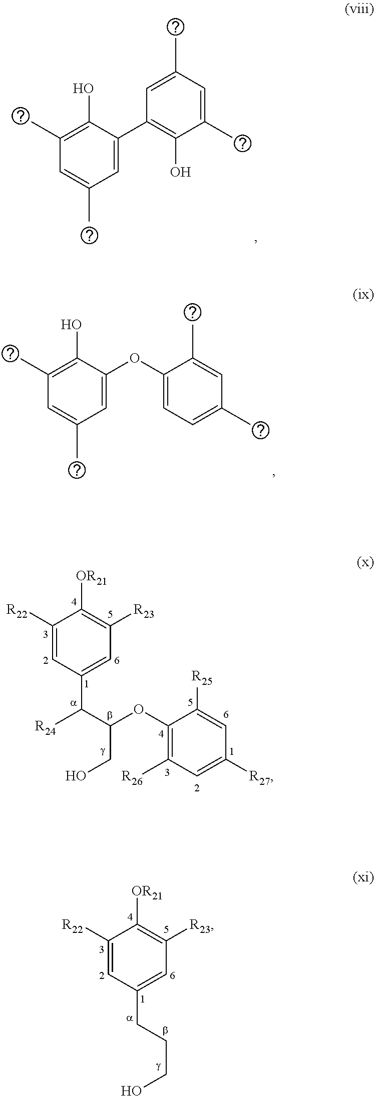

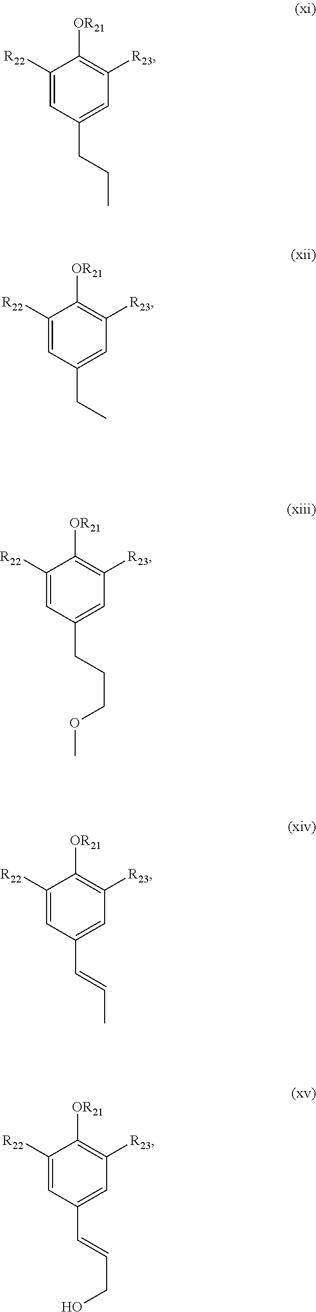

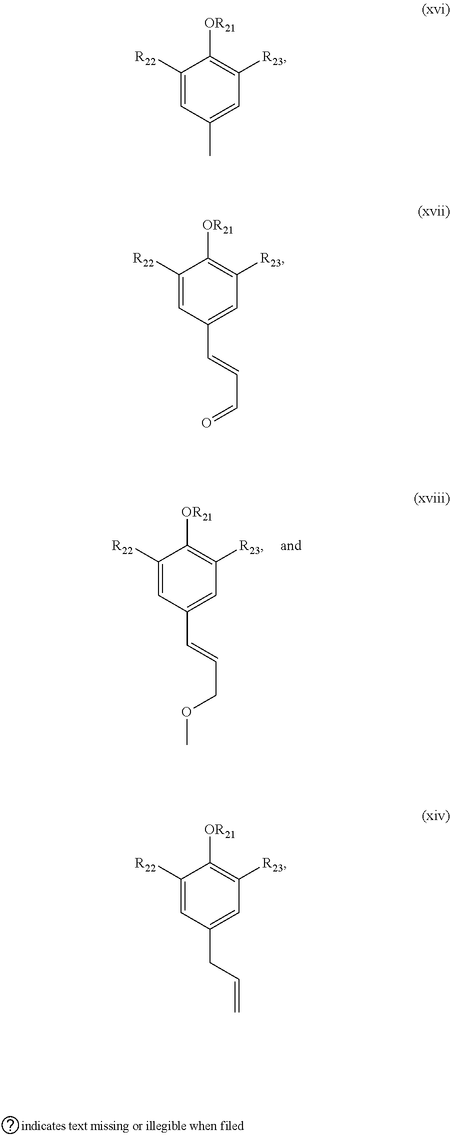

each with at least one linkage to an aromatic monomer or aromatic oligomer, and
wherein R₂₁ is independently chosen from —H, a β-O-4 linkage to an aromatic monomer, a 4-O-5 linkage to an aromatic monomer, an α-O-4 linkage to an aromatic monomer or a carbon-oxygen linkage to an aromatic monomer,
wherein each R₂₂ and R₂₃ is dependently chosen from —H, —OH, O—CH₃ or where at least R22 or R23 is independently chosen from a 4-O-5 linkage to an aromatic monomer, a 5-5 linkage to an aromatic monomer, a β-5 linkage to an aromatic monomer, a carbon linkage to an aromatic monomer, or a carbon-oxygen linkage to an aromatic monomer,
wherein R₂₄ is independently chosen from —H, OH, or —O-Alkyl wherein the alkyl group is derived from the alcohol solvent of the process,
wherein each R₂₅ and R₂₆ is independently chosen from —H, —OH, O—CH₃, a 4-O-5 linkage to an aromatic monomer, a 5-5 linkage to an aromatic monomer, a β-5 linkage to an aromatic monomer, a carbon linkage to an aromatic monomer, or a carbon-oxygen linkage to an aromatic monomer,
wherein R₂₇ is independently chosen from —H, end-unit selected from CH₃, —CH₂CH₃, —(CH₂)₂CH₃, —CH₂CH═CH₂, —CH═CHCH₃, —(CH₂)₂CH₂OH, —(CH₂)₂CHO, —CH═CHCH₂OH, —(CH₂)₂CH₂OCH₃, —CH═CHCH₂OCH₃, —(CH₂)₂CH₂OCH₂CH₃, —CH═CHCH₂OCH₂CH₃, —(CH₂)₂CH₂O(CH₂)₂CH₃, —CH═CHCH₂O(CH₂)₂CH₃, —(CH₂)₂CH₂OCH(CH₃)₂, —CH═CHCH₂OCH(CH₃)₂, —(CH₂)₂CH₂O(CH₂)₃CH₃, —CH═CHCH₂O(CH₂)₃CH₃.

In a further embodiment, the compositions described above is characterized in that the plant defense elicitor compounds comprise, or essentially consist of or consist of at least one aromatic compound selected from the formulae

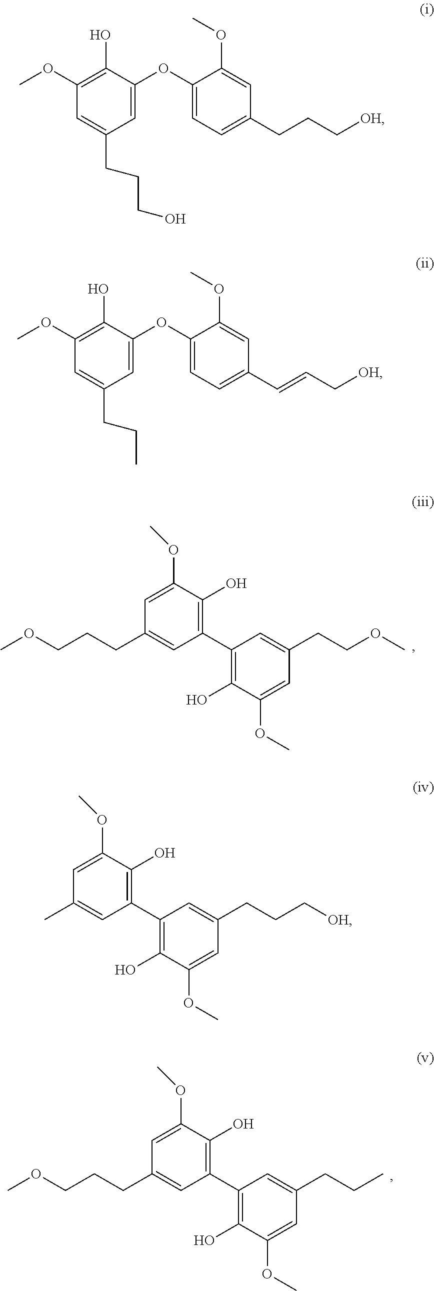

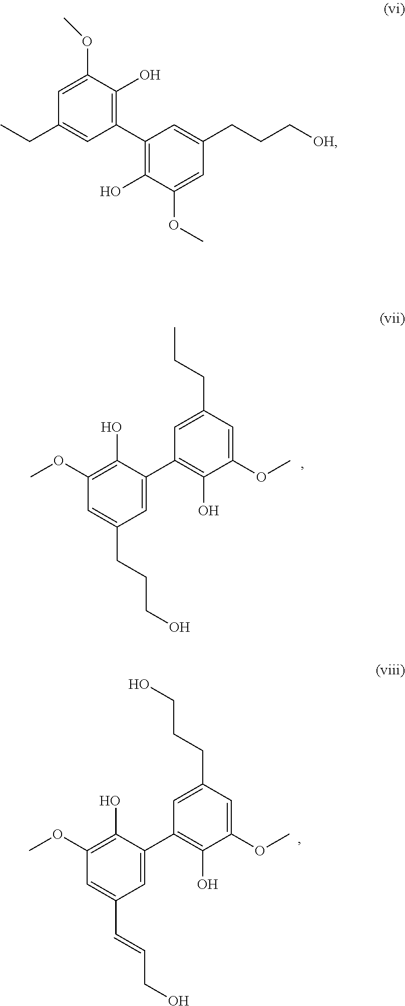

or a combination thereof.

In a further embodiment, the compositions described above is characterized in that the plant defense elicitor compounds are phenolic oligomers comprising two benzene rings directly bridged or bridged with a common bridging group of the group consisting of aliphatic chains, alkene groups, carbonyl groups and ether linkages or wherein the phenolic oligomers have two aromatic groups.

In a further embodiment, the compositions described above are characterized in that the plant defense elicitor compounds are phenolics oligomers comprising two benzene rings directly bridged or bridged with a common bridging group of the group consisting of —CH₂— groups, —CH═CH—, —C(═O)-and —O—.

In a further embodiment, the compositions described above are characterized in that the plant defense elicitor compounds are 0.5 to 30 wt % by dry weight of aromatic compounds, preferably 1 to 20 wt % by dry weight of aromatic compounds, more preferably 2 to 10 wt % of the composition in dry state.

In another aspect, the phytopharmaceutical or agrochemical composition hereof further comprises an ingredient of the group consisting of a surfactant, a biosurfactant, a penetration enhancer, a dispersing agent, an emulsifier and a carrier or a combination thereof.

In yet another aspect, the phytopharmaceutical or agrochemical composition hereof further comprises a polymerization inhibitor of the group of a carbocation scavenger, aromatic scavengers and a polyhydric alcohol or a combination thereof.

In yet another aspect, the phytopharmaceutical or agrochemical composition hereof further comprises a polymerization inhibitor wherein the repolymerization inhibitor is a compound of the group consisting of citric acid, salicylic acid, 2-naphthol, phenolic acids (e.g., vanillic acid, syringic acid), ethylene glycol, glycerol, mannitol (C₆H1₄O₆), sorbitol (C₆H₁₄O₆), xylitol (C₅H₁₂O₅), erythritol, maltitol (C₁₂H₂₄O₁₁).

In yet another aspect, the phytopharmaceutical or agrochemical composition hereof further comprises stabilizing agents of the group consisting of 1,4-butanediol, 2-hydroxy-1-naphthoic acid, 2-naphthol, 2-naphthol-7-sulfonat, 2-naththol, 3-hydroxy-2-naphthoic acid, 4-hydroxybenzoic acid, 6-hydroxy-2-naphthoic acid, ascorbic acid, bovine serum albumin, citric acid, salicylic acid, cysteine, dimethylphloroglucinol, histidine, mannitol, o-dihydroxybenzene, p-benzenediol, phloroglucinol, resorcinol, soy protein isolate, syringic acid and vanillic acid so to prevent repolymerization.

In yet another aspect, the phytopharmaceutical or agrochemical composition hereof further comprises a second plant defense elicitor of the group consisting of alginate, hexokinase, laminarin, sodium silicate, silicon, oligo-galacturonan, cellodextrin and/or chito-oligosaccharide or a plant defense elicitor selected from the group consisting of pectin fragment, oligogalacturonide, cellobiose, xyloglucan, non-branched P-1,3-glucan, chitin fragment, arabinose, arabinan, rhamnose, homogalacturonan, rhamnogalacturonan I and II, xylogalacturonan and starch or combinations thereof.

In yet another aspect, the phytopharmaceutical or agrochemical composition hereof further comprises a fungicide, an antimicrobial, an insecticidal, and/or an antiviral for instance.

In a practical embodiment, the phytopharmaceutical or agrochemical composition hereof comprises the lignin-derived oligomer aromatics with a DP of 2 to 8 (preferably 2-4) from lignin that is from coconut husk, softwood trees, hardwood trees, a grass (such as bamboo, corn stalks and stover; wheat straw, rice straw, barley straw, miscanthus), flax shives or hemp stalk or a combination thereof.

In another practical embodiment, the phytopharmaceutical or agrochemical composition hereof comprises an ingredient of the group consisting of a surfactant, a biosurfactant, a penetration enhancer, a dispersing agent, an emulsifier and a carrier or a combination thereof.

In yet another practical embodiment, the phytopharmaceutical or agrochemical composition hereof further comprises a second plant defense elicitor, e.g., of the group consisting of alginate, hexokinase, laminarin, sodium silicate, silicon, oligo-galacturonan, cellodextrin and/or chito-oligosaccharide or a plant defense elicitor selected from the group consisting of pectin fragment, oligogalacturonide, cellobiose, xyloglucan, non-branched P-1,3-glucan, chitin fragment, arabinose, arabinan, rhamnose, homogalacturonan, rhamnogalacturonan I and II, xylogalacturonan, starch, and combinations thereof.

In yet another practical embodiment, the phytopharmaceutical or agrochemical composition hereof further comprises a fungicide, an antimicrobial, an insecticidal, or an antiviral.

In yet another practical embodiment, the phytopharmaceutical or agrochemical composition hereof further comprises a fungicide selected from the group consisting of phosphonates, benzamides, carbamates, dithiocarbamates, phthalimides, triazoles, quinolines, sulfur, and cyanoimidazoles or a fungicide selected from the group consisting of 2,6-dichloro-N-[3-chloro-5-(trifluoromethyl)-2-pyridinylmethyl]benzamide; propyl 3-(dimethylamino)propylcarbamate hydrochloride; (2RS,3SR)-1-[3-(2-chlorophenyl)-2,3-epoxy-2-(4-fluorophenyl)propyl]-1H-1,2,4-triazole; 5,7-dichloro-4-quinolyl 4-fluorophenyl ether; sulfur; 4-chloro-2-cyano-N,N-dimethyl-5-(4-methylphenyl)-1H-imidazole-1-sulfonamide; N-(trichloromethylthio)phthalimide; manganese ethylenebis(dithiocarbamate) (polymeric) complex with zinc salt and methyl (E)-methoxyimino-{(E)-α-[1-(α,α,α-trifluoro-m-tolyl)ethylideneaminooxy]-o-tolyl}acetate; or a combination thereof.

In another aspect, provided is a phytopharmaceutical or agrochemical composition hereof further comprises stabilizing agents of the group consisting of 1,4-butanediol, 2-hydroxy-1-naphthoic acid, 2-naphthol, 2-naphthol-7-sulfonat, 2-naththol, 3-hydroxy-2-naphthoic acid, 4-hydroxybenzoic acid, 6-hydroxy-2-naphthoic acid, ascorbic acid, bovine serum albumin, citric acid, salicylic acid, cysteine, dimethylphloroglucinol, histidine, mannitol, o-dihydroxybenzene, p-benzenediol, phloroglucinol, resorcinol, soy protein isolate, syringic acid and vanillic acid so to prevent repolymerization.

In another aspect, provided is a phytopharmaceutical or agrochemical composition hereof that comprises a) the plant defense elicitor of the disclosure and as described hereabove and b) a fungicide, e.g., a fungicide selected from the group consisting of selected from the group comprising: phosphonates, benzamides, carbamates, dithiocarbamates, phthalimides, triazoles, quinolines, sulfur, and cyanoimidazoles or a fungicide selected from the group consisting of 2,6-dichloro-N-[3-chloro-5-(trifluoromethyl)-2-pyridinylmethyl]benzamide; propyl 3-(dimethylamino)propylcarbamate hydrochloride; (2RS,3SR)-1-[3-(2-chlorophenyl)-2,3-epoxy-2-(4-fluorophenyl)propyl]-1H-1,2,4-triazole; 5,7-dichloro-4-quinolyl 4-fluorophenyl ether; sulfur; 4-chloro-2-cyano-N,N-dimethyl-5-(4-methylphenyl)-1H-imidazole-1-sulfonamide; N-(trichloromethylthio)phthalimide; manganese ethylenebis(dithiocarbamate) (polymeric) complex with zinc salt and methyl (E)-methoxyimino-{(E)-α-[1-(α,α,α-trifluoro-m-tolyl)ethylideneaminooxy]-o-tolyl}acetate; or a combination thereof.

In another aspect, provided is a phytopharmaceutical or agrochemical composition hereof that also comprises another type of plant defense elicitor such as alginate, hexokinase, laminarin, sodium silicate, silicon, oligo-galacturonan, cellodextrin and/or chito-oligosaccharide or a plant defense elicitor selected from the group consisting of pectin fragment, oligogalacturonide, cellobiose, xyloglucan, non-branched P-1,3-glucan, chitin fragment, arabinose, arabinan, rhamnose, homogalacturonan, rhamnogalacturonan I and II, xylogalacturonan, starch, and combinations thereof.

In another aspect, provided is a phytopharmaceutical or agrochemical composition hereof that further comprises a co-formulant selected from the group comprising: detergents, emulsifiers, dispersing agents, anti-foaming agents, penetration enhancers, humectants, wetting agents of ionic or non-ionic type, anti-freeze agents, preservative agents, absorbent agents, thickeners, buffers, sticker agents, diluents or a mixture thereof, preferably a surfactant selected from the group comprising: detergents, emulsifiers, dispersing agents, anti-foaming agents, penetration enhancers, humectants or wetting agents of ionic or non-ionic type, or a mixture thereof.

In another aspect, provided is a phytopharmaceutical or agrochemical composition hereof that further comprises a surfactant comprising one or more of the following components: castor oil ethoxylate, rapeseed methyl ester, alkyl phosphates, tributyl phosphate, tripropyl phosphate, naphthalenesulphonic acid salts, a combination of organic sulfonate and 2-methylpentane-2,4-diol, alkylpolyglucoside, siloxanes derivates, alkylsulfonates, polycarboxylates, lignosulfonates, alkoxylated triglycerides, fatty amines polymers, dioctylsulfosuccinates or polyoxyethylene (20) sorbitan monolaurate, preferably C18-castor-oil-ethoxylate, a combination of organic sulfonate and 2-methylpentane-2,4-diol, or polyoxyethylene (20) sorbitan monolaurate.

Described is a method of activating plant defense against a pathogen stressor and/or abiotic stressor, comprising contacting the plant with an effective amount of a lignin-derived aromatic oligomers with a DP of 2 to 8 (preferably 2-4), or a synthesized structurally similar compound. This embodiment of the disclosure advantageously comprises activating plant defense preventive or activating plant defense in case of a stressor plant attack. In an aspect, the disclosure provides that the lignin-derived aromatic oligomers are obtained from a lignin that is depolymerized or decomposed by reductive catalytic fractionation (RCF) of lignin or lignocellulose. This embodiment of the disclosure advantageously provides very stable lignin-derived aromatic oligomers oils, even at room temperature and when no re-polymerization inhibitors are added. In another application, the method according to the disclosure activating plant defense against a pathogen stressor and/or abiotic stressor provides that the lignin-derived aromatic oligomers are from a lignin that is depolymerized or decomposed by non-catalytic thermosolvolytic depolymerization of lignin or lignocellulose.

Some of the methods described herein may be embodied where the lignin-derived aromatic oligomers comprise, consist essentially of, or consist of I) at least one aromatic compound selected from the formulae

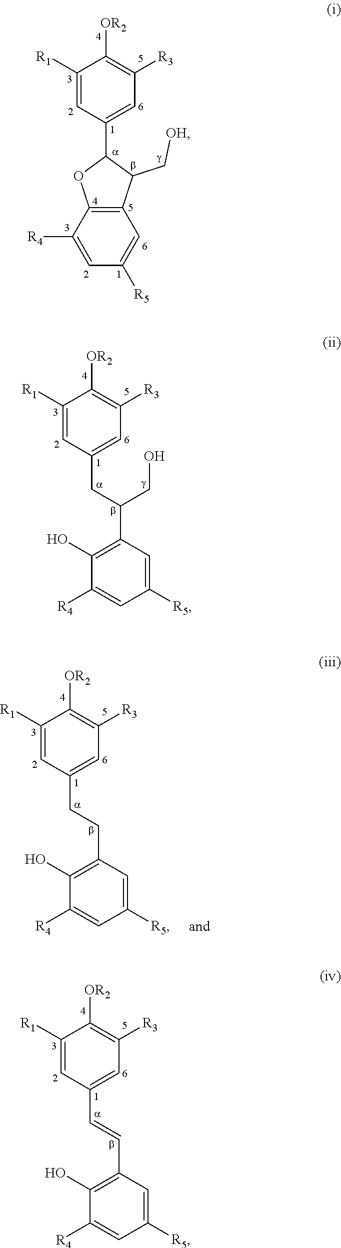

and
wherein each R₁, R₃, and R₄ is independently chosen from —H, —OH, O—CH₃, a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or aromatic oligomer, or a carbon-oxygen linkage to an aromatic monomer or aromatic oligomer,
wherein R₂ is —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer, or a carbon-oxygen linkage to an aromatic monomer or aromatic oligomer,
wherein R₅ is selected from —H, a β-O-4 linkage to an aromatic monomer or aromatic oligomer, a β-5 linkage to an aromatic monomer or aromatic oligomer, a β-β linkage to an aromatic monomer or aromatic oligomer, a β-1 linkage to an aromatic monomer or aromatic oligomer, an end-unit selected from CH₃, —CH₂CH₃, —(CH₂)₂CH₃, —CH₂CH═CH₂, —CH═CHCH₃, —(CH₂)₂CH₂OH, —(CH₂)₂CHO, —CH═CHCH₂OH, —(CH₂)₂CH₂OCH₃, —CH═CHCH₂OCH₃, —(CH₂)₂CH₂OCH₂CH₃, —CH═CHCH₂OCH₂CH₃, —(CH₂)₂CH₂O(CH₂)₂CH₃, —CH═CHCH₂O(CH₂)₂CH₃, —(CH₂)₂CH₂OCH(CH₃)₂, —CH═CHCH₂OCH(CH₃)₂, —(CH₂)₂CH₂O(CH₂)₃CH₃, —CH═CHCH₂O(CH₂)₃CH₃, a carbon linkage to an aromatic monomer or aromatic oligomer;
II) wherein the aromatic compounds comprise at least one aromatic compound selected from the formulae

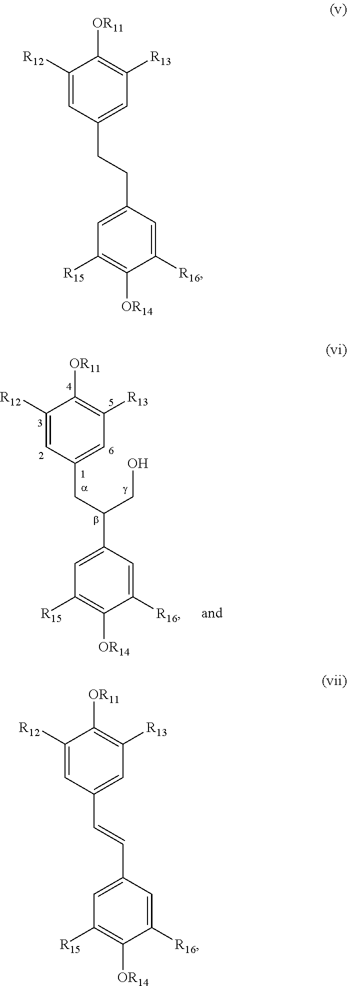

and
wherein each R₁₂, R₁₃, R₁₅ and R₁₆ is independently chosen from —H, —OH, O—CH₃, a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or aromatic oligomer, or a carbon-oxygen linkage to an aromatic monomer or aromatic oligomer,
wherein R₁₁ and R₁₄ is independently chosen from —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer or a carbon-oxygen linkage to an aromatic monomer or aromatic oligomer, and/or
III) wherein at the aromatic compounds comprise at least one aromatic compound selected from the formula

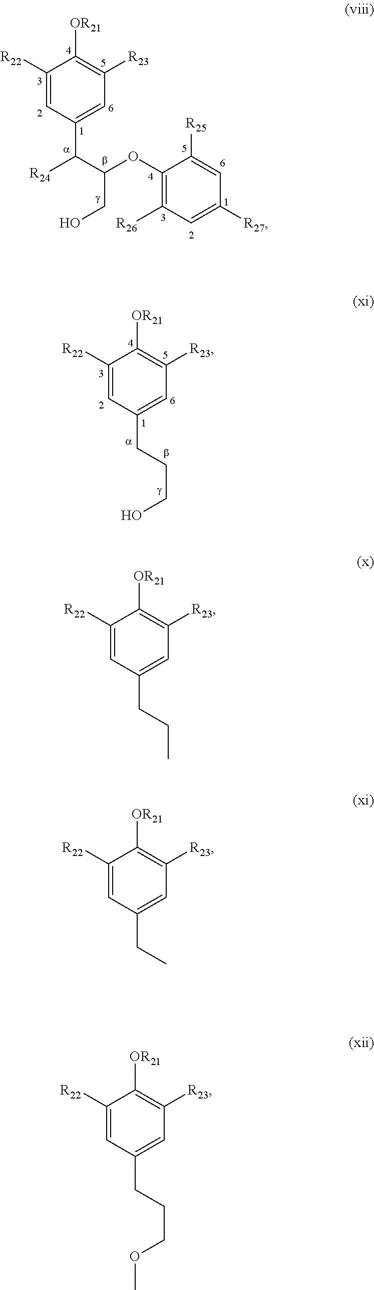

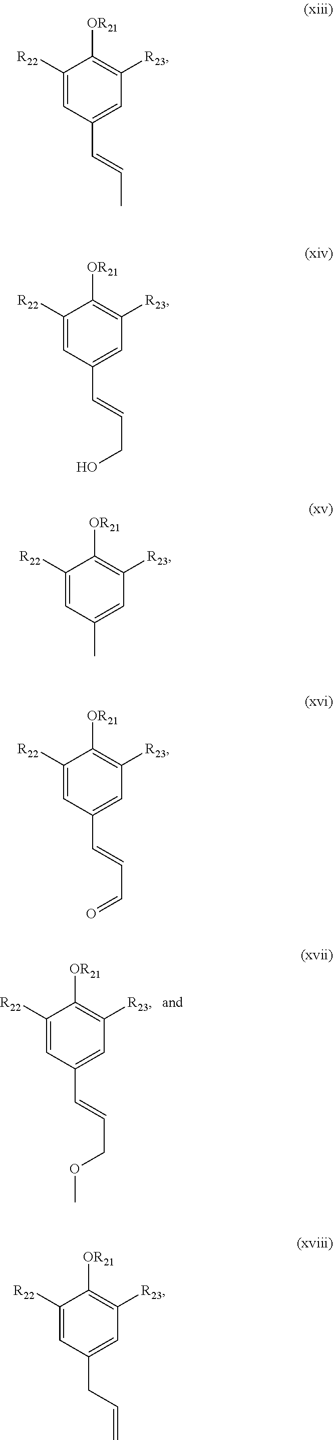

each with at least one linkage to an aromatic monomer or aromatic oligomer and
wherein R₂₁ is independently chosen from —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer or a carbon-oxygen linkage to an aromatic monomer or aromatic oligomer,
wherein each R₂₂ and R₂₃ is dependently chosen from —H, —OH, O—CH₃ or from a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or an aromatic oligomer, or a carbon-oxygen linkage to an aromatic monomer or aromatic oligomer,
wherein R₂₄ is independently chosen from —H, OH, or —O-Alkyl wherein the alkyl group is derived from the alcohol solvent of the process,
wherein each R₂₅ and R₂₆ is independently chosen from —H, —OH, O—CH₃, a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or an aromatic oligomer, or a carbon-oxygen linkage to an aromatic monomer or aromatic oligomer,
wherein R₂₇ is independently chosen from —H, a β-O-4 linkage to an aromatic monomer or aromatic oligomer, a β-5 linkage to an aromatic monomer or aromatic oligomer, a β-β linkage to an aromatic monomer or aromatic oligomer, a β-1 linkage to an aromatic monomer or aromatic oligomer, end-unit selected from CH₃, —CH₂CH₃, —(CH₂)₂CH₃, —CH₂CH═CH₂, —CH═CHCH₃, —(CH₂)₂CH₂OH, —(CH₂)₂CHO, —CH═CHCH₂OH, —(CH₂)₂CH₂OCH₃, —CH═CHCH₂OCH₃, —(CH₂)₂CH₂OCH₂CH₃, —CH═CHCH₂OCH₂CH₃, —(CH₂)₂CH₂O(CH₂)₂CH₃, —CH═CHCH₂O(CH₂)₂CH₃, —(CH₂)₂CH₂OCH(CH₃)₂, —CH═CHCH₂OCH(CH₃)₂, —(CH₂)₂CH₂O(CH₂)₃CH₃, —CH═CHCH₂O(CH₂)₃CH₃, a carbon linkage to an aromatic monomer or an aromatic oligomer.

Some of the methods described herein may be embodied as that the lignin-derived aromatic oligomers comprise, or essentially consist of or consist of I) at least one aromatic compound selected from the formulae

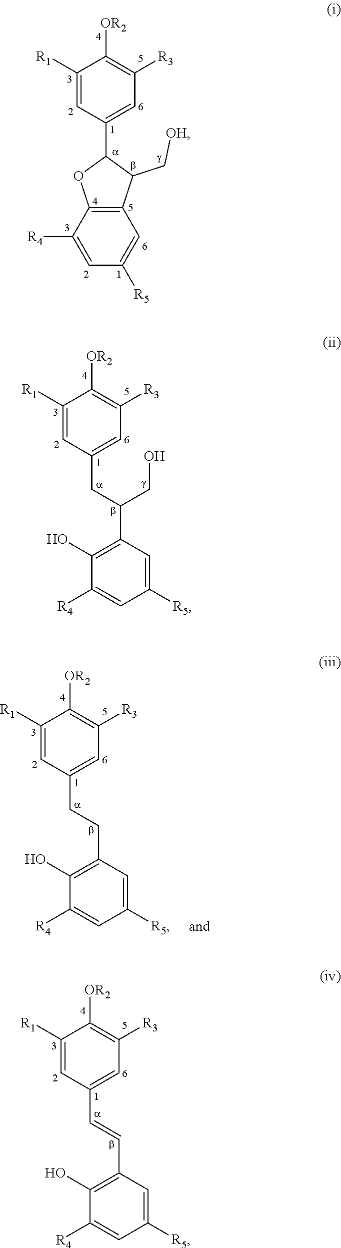

and
wherein each R₁, R₃, and R₄ is independently chosen from —H, —OH, O—CH₃,
wherein R₂ is —H,
wherein R₅ is selected from —H, an end-unit selected from CH₃, —CH₂CH₃, —(CH₂)₂CH₃, —CH₂CH═CH₂, —CH═CHCH₃, —(CH₂)₂CH₂OH, —(CH₂)₂CHO, —CH═CHCH₂OH, —(CH₂)₂CH₂OCH₃, —CH═CHCH₂OCH₃, —(CH₂)₂CH₂OCH₂CH₃, —CH═CHCH₂OCH₂CH₃, —(CH₂)₂CH₂O(CH₂)₂CH₃, —CH═CHCH₂O(CH₂)₂CH₃, —(CH₂)₂CH₂OCH(CH₃)₂, —CH═CHCH₂OCH(CH₃)₂, —(CH₂)₂CH₂O(CH₂)₃CH₃, —CH═CHCH₂O(CH₂)₃CH₃,
II) wherein the aromatic compounds comprise at least one aromatic compound selected from the formulae

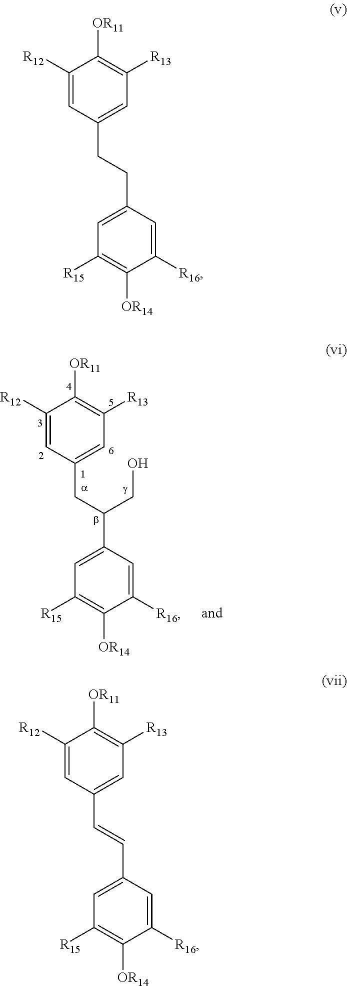

and
wherein each R₁₂, R₁₃, R₁₅ and R₁₆ is independently chosen from —H, —OH, O—CH₃,
wherein R₁₁ and R₁₄ is independently chosen from —H,
III) wherein at the aromatic compounds comprise at least one aromatic compound selected from the formula (viii)

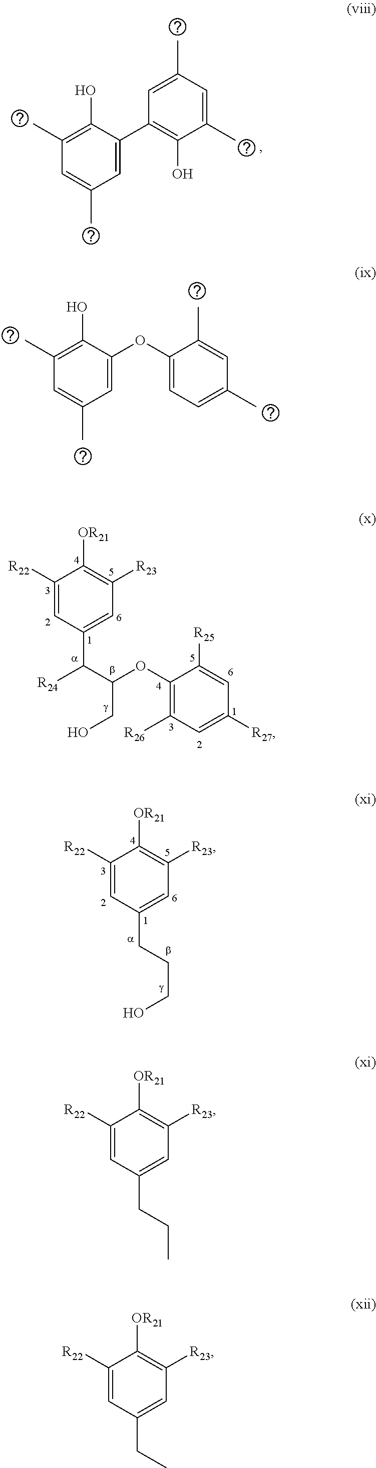

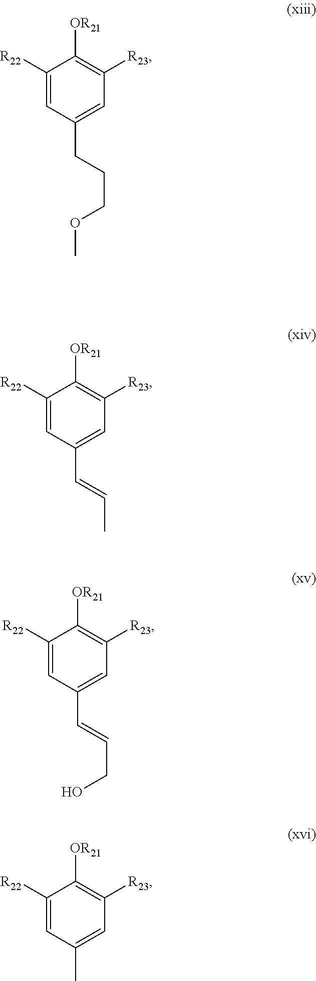

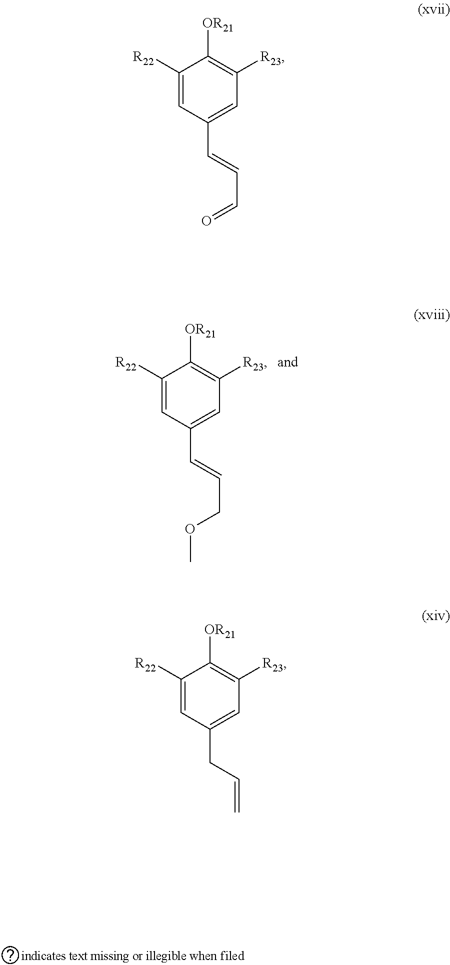

each with at least one linkage to an aromatic monomer or aromatic oligomer, and
wherein R₂₁ is independently chosen from —H, a β-O-4 linkage to an aromatic monomer, a 4-O-5 linkage to an aromatic monomer, an α-O-4 linkage to an aromatic monomer or a carbon-oxygen linkage to an aromatic monomer,
wherein each R₂₂ and R₂₃ is dependently chosen from —H, —OH, O—CH₃ or where at least R₂₂ or R₂₃ is independently chosen from a 4-O-5 linkage to an aromatic monomer, a 5-5 linkage to an aromatic monomer, a β-5 linkage to an aromatic monomer, a carbon linkage to an aromatic monomer, or a carbon-oxygen linkage to an aromatic monomer,
wherein R₂₄ is independently chosen from —H, OH, or —O-Alkyl wherein the alkyl group is derived from the alcohol solvent of the process,
wherein each R₂₅ and R₂₆ is independently chosen from —H, —OH, O—CH₃, a 4-O-5 linkage to an aromatic monomer, a 5-5 linkage to an aromatic monomer, a β-5 linkage to an aromatic monomer, a carbon linkage to an aromatic monomer, or a carbon-oxygen linkage to an aromatic monomer,
wherein R₂₇ is independently chosen from —H, end-unit selected from CH₃, —CH₂CH₃, —(CH₂)₂CH₃, —CH₂CH═CH₂, —CH═CHCH₃, —(CH₂)₂CH₂OH, —(CH₂)₂CHO, —CH═CHCH₂OH, —(CH₂)₂CH₂OCH₃, —CH═CHCH₂OCH₃, —(CH₂)₂CH₂OCH₂CH₃, —CH═CHCH₂OCH₂CH₃, —(CH₂)₂CH₂O(CH₂)₂CH₃, —CH═CHCH₂O(CH₂)₂CH₃, —(CH₂)₂CH₂OCH(CH₃)₂, —CH═CHCH₂OCH(CH₃)₂, —(CH₂)₂CH₂O(CH₂)₃CH₃, —CH═CHCH₂O(CH₂)₃CH₃.

Some of the methods described herein may be embodied as that the lignin-derived aromatic oligomers comprise, or essentially consist of or consist of at least one aromatic compound selected from the formulae (i)

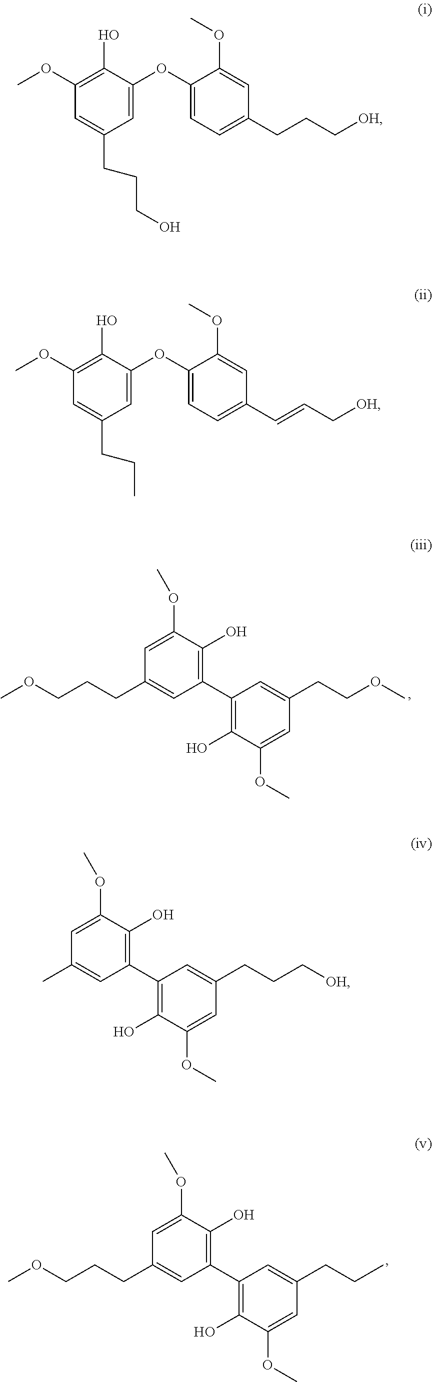

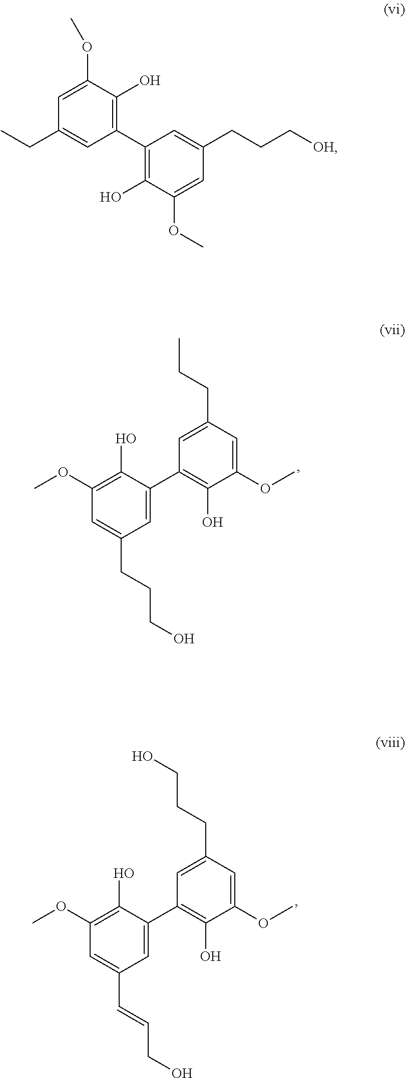

or a combination thereof.

Particular and preferred aspects of the disclosure are set out in the accompanying independent and dependent claims. Features from the dependent claims may be combined with features of the independent claims and with features of other dependent claims as appropriate and not merely as explicitly set out in the claims.

Thus, the claims following the detailed description are hereby expressly incorporated into this detailed description, with each claim standing on its own as a separate embodiment of this disclosure.

### BRIEF DESCRIPTION OF THE DRAWINGS

The disclosure will become more fully understood from the detailed description given hereinbelow and the accompanying drawings which are given by way of illustration only, and thus are not limitative of the disclosure.

A figure can contain different panels, e.g., FIG. 1 contains panel A, B, C, which can be marked FIG. 1A, FIG. 1B and FIG. 1C. “Pine” refers to biomass from the Pine family (Pinaceae).

FIGS. 1A-1C are schematic diagrams showing depolymerized lignin extraction from biomass through reductive catalytic fractionation (RCF) (FIG. 1A), with use of heterogenous catalysts such as ruthenium or palladium on carbon with use of an organic solvent (or mixture) in a reductive environment, and non-catalytic thermosolvolytic fractionation (FIG. 1B) without catalyst and under inert atmosphere. Subsequent fractionation of the lignin oil by liquid-liquid extraction is depicted in FIG. 1C.

FIGS. 2A-2E are graphs showing the gel permeation chromatography (GPC) of depolymerized lignin oil and fractions thereof obtained through liquid-liquid extraction from pine and poplar biomass obtained through RCF in FIGS. 2A, 2B, 2C and 2D or in a reductive environment or a non-catalytic thermosolvolytic fractionation of pine biomass without catalyst and under inert atmosphere in FIG. 2E. The RCF and non-catalytic lignin oil (fractions) are named after i) the biomass used (e.g., poplar, FIGS. 2A and 2B; or pine, FIGS. 2C, 2D, 2E) ii) the presence of a catalyst used during the lignin extraction process or not as indicated by the acronym of the catalyst (e.g., Ru (FIGS. 2A and 2C) and Pd (FIGS. 2B and 2D) for heterogenous catalyst containing ruthenium or palladium, respectively), iii) the final solvent composition used during subsequent liquid-liquid extraction, being a mixture of heptane and ethyl acetate in a certain ratio (% v/v, e.g., 80% heptane and 20% ethyl acetate, H80E20) and iv) whether the obtained fraction is either the dissolved liquid (“L”) or the residue (“R”) when using the respective solvent composition. For example, the lignin oil obtained using pine biomass with ruthenium catalyst in a RCF reaction and the obtained residue thereof by subsequent fractionation with 100% heptane is called PineRuH100R.

FIGS. 3A-3C are bar graphs that show in FIG. 3A the relative pathogen proliferation of Hyaloperonospora arabidopsidis in A. thaliana plants treated with reductive catalytic fractionation (RCF) lignin oil or fractions thereof (H100R or H100L) from two different biomass types (poplar and pine) using two different heterogenous catalysts (ruthenium, Ru, and palladium, Pd). In FIG. 3B, a dose study on the plant disease resistance-inducing capabilities of the lignin-derived di/triphenolics fraction heptane insoluble fraction (PineRuH100R) from RCF of pine biomass. FIG. 3C is a graphic display of a dose-response study on the plant disease resistance-inducing capabilities of PineRuH100R=compared to the PimeRu80E20L fraction obtained through liquid-liquid extraction of PineRuH100R (FIG. 1C). On A. thaliana after the treatment with the lignin derivative H. arabidopsidis pathogen spores were used as shown in FIG. 3A to identify the most active lignin fraction; FIG. 3B to optimize the treatment concentration; and in FIG. 3C identify the active IR-eliciting dimers within the PineRuH100R fraction. Therefore, A. thaliana seedlings were treated with RCF extracts 8 days post seeding by spraying plant leaves until run-off. 3 days post treatment, plants were harvested, subsequently genomic DNA was extracted and qPCR was done to determine the disease index. Treatment concentration in panel 3A was 1 mg/ml. The concentration of the PineRuH80E20L subfraction of the PineRuH100R (FIG. 1C) is adjusted based on the relative weight of the fractions obtained after liquid-liquid extraction from the PineRuH100R, in this case being 0.238 mg/ml. *p<0.05; **p<0.01; ***p<0.001; ****p<0.0001.

FIGS. 4A-4C are bar graphs showing the IR activity of the heptane insoluble fraction from a depolymerized lignin obtained from the catalytic RCF process (PineRuH100R) in comparison to the heptane insoluble fraction and a non-catalytic thermosolvolytic fractioning process (PineH100R) both emulsified in 1% v/v DMSO at a concentration of 1 mg/ml prior to immediate testing (FIG. 4A) or after an accelerated stability test at higher temperatures for a shorter period of time (FIG. 4B, 30 days 54° C.) and stability test at lower temperature for a prolonged period of time (FIG. 4C, 58 days, 4° C.). It concerns stability tests with lignin oil in solution/emulsified form in 1% v/v DMSO at 54° C. (accelerated) and at 4° C. with subsequent testing of the ISR activity. A. thaliana is treated with the H. arabidopsidis pathogen spores. FIG. 4A shows induced plant disease resistance activity of the depolymerized lignin fraction of the non-catalytic thermosolvolytic fractioning process (PineH100R) and of the RCF process (PineRuH100R); FIG. 4B shows the difference in stability of the PineRuH100R and PineH100R with the lignin-derived di/triphenolics after storage for 30 days at 54° C. and FIG. 4C 58 days at 4° C. It can be concluded that the disease resistance-inducing activity was initially observed, however in solution/emulsification of 1% v/v DMSO at 4° C. for 58 days or at 54° C. for 30 days, the plant disease resistance potential is clearly absent in the lignin oil fraction obtained from the non-catalytic thermosolvolytic fractioning process (PineH100R) compared to the one from the catalytic RCF process (PineRuH100R) which maintained its activity. A repolymerization stabilizer needs to be incorporated in the final formulation to maintain the plant disease resistance potential of the depolymerized lignin oil from the non-catalytic thermosolvolytic fractioning process. PineRuH100R shows higher stability than the non-catalytic thermosolvolytic fractioning equivalent PineH100R, when kept at similar conditions at 54° C. for 30 days and 4° C. for 58 days.

FIG. 5 is a bar graph showing infection of A. thaliana by H. arabidopsidis pathogen to determine optimal time between treatment by the plant disease resistance inducer comprises lignin-derived di/triphenolics (PineRuH100R from RCF of pine biomass) and infection. Based on this data we concluded that the biggest relative reduction in pathogen proliferation is observed when plants are treated 24 hours before exposure to H. arabidopsidis.

FIGS. 6A and 6B are bar graphs displaying in FIG. 6A the plant disease resistance activity of various tomato (Solanum lycopersicum) cultivars after treatment with PineRuH100R (from RCF of pine biomass), which comprises lignin-derived diphenolics, triphenolics and tetraphenolics, and infection with Botrytis cinerea. Solid, darker bars are the infected tomato cultivars that were not treated with the PineRuH100R which comprises lignin-derived diphenolics, triphenolics and tetraphenolics. FIG. 6B shows that IR induction is at least maintained for 12 days post PineRuH100R treatment (dpt) in S. lycopersicum when infected with B. cinerea. Briefly, 24 days post seeding, S. lycopersicum plants were treated with PineRuH100R by spraying the leaves with compound solution until run-off. Treatment with the solvent 1% v/v DMSO was included as mock treatment (indicated with “-”). Three, twelve and seventeen days after treatment, five leaflets per plant were inoculated with 5 μl droplets of a B. cinerea R16 strain spore suspension of 5×10⁵ spores/ml in potato dextrose broth. The hydroponics tanks were placed inside an infection box, containing a moist mat to obtain high humidity, in the growth chamber. The disease symptoms were quantified by measuring the diameter parallel to the midrib of the developing necrotic lesions at 2 dpi. The lesion area was calculated using the formula below. Lesion area=(Lesion diameter/2)\hat{ }2*π. *p<0.05; **p<0.01; ***p<0.001; ****p<0.0001.

FIG. 7 is a bar graph concerning induction by RCF lignin of increased resistance in tomato (S. lycopersicum) against insects and quantifies the number of necrotic rings caused by Nesidiocoris tenuis (also known as the tomato bug, tobacco leaf bug, tomato mirid, or green tobacco capsid) feeding post mock (1% v/v DMSO) and PineRuH100R (1 mg/ml, emulsified in 1% v/v DMSO) treatment in accordance with Example 9. It is clearly demonstrated that the treatment with the depolymerized lignin is protective against insect feeding on tomato plants.

FIG. 8 is a bar graph displaying the difference in tomato transpiration rate between mock and PineRuH100R sprayed plants under heat stress conditions (38° C.), in a treatment in accordance with Example 10.

FIG. 9 is a bar graph showing the seedling survival post mock (1% v/v DMSO) and PineRuH100R (1 mg/ml in 1% v/v DMSO) 6 days after heat stress exposure. The PineRuH100R renders the seedlings more protected to heat stress and had a higher survival rate.

FIG. 10 is a schematic diagram showing depolymerized lignin extraction from biomass through oxidative catalytic fractionation (OCF), with use of heterogenous catalysts with use of an alkaline aqueous solution in an oxidative environment. In Example 11, the heterogenous catalysts, CuO, is used as heterogenous catalysts for OCF on birch wood at 160° C.

FIGS. 11A and 11B show the A. thaliana (ecotype: Columbia-0) plants seedlings in little pots (20 well-developed, freestanding seedlings) treated after eight days after sowing treated with mock (1% v/v DMSO) (FIG. 11A.), and depolymerized lignin monomers from OCF (OCF 4.1, 1 mg/ml) (FIG. 11B.) fraction by spraying the leaves with compound solution until run-off. The figures clearly show the toxic impact of the monomers from OCF (OCF 4.1) on the plants.

FIG. 12 shows the relative pathogen proliferation of H. arabidopsidis in A. thaliana plants treated with the heptane insoluble fraction from a depolymerized lignin obtained from the catalytic RCF process of pine wood (PineRuH100R) or OCF oligomers (OCF 4.2) obtained from birch wood using a catalytic OCF process demonstrating equal elicitor activity of depolymerized lignin oligomers (DP≥2) from RCF and OCF.

### ABBREVIATIONS IN THE FIGURES

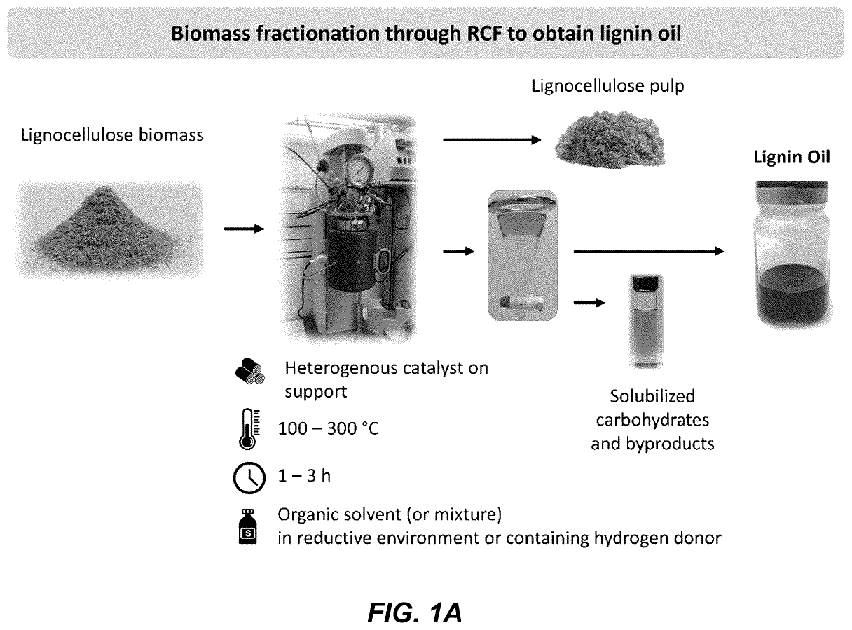

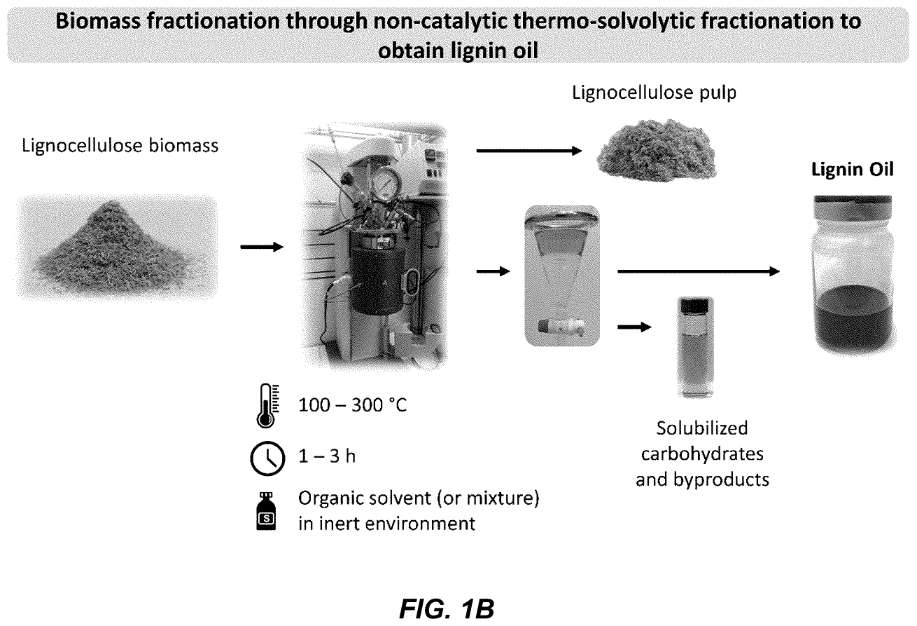

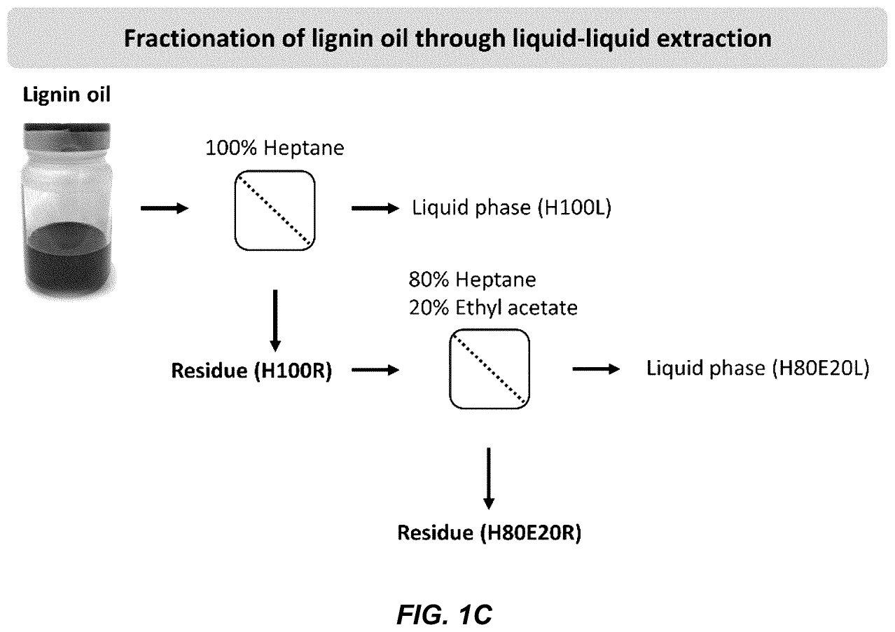

H80E20L=the dissolved liquid (“L”) subfraction (of the H100R) obtained in the fractioning of lignin oil through liquid-liquid extraction from mixture of heptane and ethyl acetate in a certain ratio (% v/v, e.g., 80% heptane and 20% ethyl acetate), see FIG. 1C.

H80E20R=the residue (“R”) subfraction (of the H100R) obtained in the fractioning of lignin oil through liquid-liquid extraction from mixture of heptane and ethyl acetate in a certain ratio (% v/v, e.g., 80% heptane and 20% ethyl acetate), see FIG. 1C.

PineH100R=the depolymerized lignin (lignin oil) residue (“R”) fraction from the 100% heptane extraction of the non-catalytic thermosolvolytic fractioning process using pine biomass, see FIG. 1C.

PineH100L=the depolymerized lignin (lignin oil) dissolved liquid (“L”) fraction from the 100% heptane extraction of the non-catalytic thermosolvolytic fractioning process using pine biomass.

PineRuH100R=the depolymerized lignin (lignin oil) residue (“R”) fraction from the 100% heptane extraction of the reductive catalytic fractionation (RCF) process using pine biomass with ruthenium catalyst, see FIG. 1C.

PineCdH100R=the depolymerized lignin (lignin oil) residue (“R”) fraction from the 100% heptane extraction of the reductive catalytic fractionation (RCF) process using pine biomass with cadmium catalyst, see FIG. 1C.

PineRuH80E20L=the dissolved liquid (“L”) subfraction (of the PineRuH100R) obtained in the fractioning of lignin oil through liquid-liquid extraction from mixture of heptane and ethyl acetate in a certain ratio (% v/v, e.g., 80% heptane and 20% ethyl acetate), see FIG. 1C.

PineCdH80E20L=the dissolved liquid (“L”) subfraction (of the PineCdH100R) obtained in the fractioning of lignin oil through liquid-liquid extraction from mixture of heptane and ethyl acetate in a certain ratio (% v/v, e.g., 80% heptane and 20% ethyl acetate), see FIG. 1C.

PopH100R=the depolymerized lignin (lignin oil) residue (“R”) fraction from the 100% heptane extraction of the non-catalytic thermosolvolytic fractioning process using poplar biomass, see FIG. 1C.

PopH100L=the depolymerized lignin (lignin oil) dissolved liquid (“L”) fraction from the 100% heptane extraction of the non-catalytic thermosolvolytic fractioning process using poplar biomass, see FIG. 1C.

PopRuH100R=the depolymerized lignin (lignin oil) residue (“R”) fraction from the 100% heptane extraction of the reductive catalytic fractionation (RCF) process using poplar biomass with ruthenium catalyst, see FIG. 1C.

PopCdH100R=the depolymerized lignin (lignin oil) residue (“R”) fraction from the 100% heptane extraction of the reductive catalytic fractionation (RCF) process using poplar biomass with cadmium catalyst, see FIG. 1C.

PopRuH80E20L=the dissolved liquid (“L”) subfraction (of the PopRuH100R) obtained in the fractioning of lignin oil through liquid-liquid extraction from mixture of heptane and ethyl acetate in a certain ratio (% v/v, e.g., 80% heptane and 20% ethyl acetate), see FIG. 1C.

PopRuOil=the lignin oil obtained by lignin depolymerized lignin by reductive catalytic fractionation (RCF) process using poplar biomass with ruthenium catalyst.

PopRuOil=the lignin oil obtained by lignin depolymerized lignin by reductive catalytic fractionation (RCF) process using poplar biomass with ruthenium catalyst.

PineRuOil=the lignin oil obtained by lignin depolymerized lignin by reductive catalytic fractionation (RCF) process using pine biomass with ruthenium catalyst.

PineRuOil=the lignin oil obtained by lignin depolymerized lignin by reductive catalytic fractionation (RCF) process using pine biomass with ruthenium catalyst.

PineOil=The lignin oil obtained by lignin depolymerized lignin by non-catalytic thermosolvolytic depolymerization.dpt=days post treatment.

### DETAILED DESCRIPTION

The following detailed description of the disclosure refers to the accompanying drawings. Also, the following detailed description does not limit the disclosure. Instead, the scope of the disclosure is defined by the appended claims and equivalents thereof.

Several documents are cited throughout the text of this specification. Each of the documents herein (including any manufacturer's specifications, instructions, etc.) are hereby incorporated by reference; however, there is no admission that any document cited is indeed prior art of the disclosure.

The disclosure will be described with respect to particular embodiments and with reference to certain drawings, but the disclosure is not limited thereto but only by the claims. The drawings described are only schematic and are non-limiting. In the drawings, the size of some of the elements may be exaggerated and not drawn to scale for illustrative purposes. The dimensions and the relative dimensions do not correspond to actual reductions to practice of the disclosure.

Furthermore, the terms “first,” “second,” “third” and the like in the description and in the claims, are used for distinguishing between similar elements and not necessarily for describing a sequential or chronological order. It is to be understood that the terms so used are interchangeable under appropriate circumstances and that the embodiments of the disclosure described herein are capable of operation in other sequences than described or illustrated herein.

It is to be noticed that the term “comprising,” used in the claims, should not be interpreted as being restricted to the means listed thereafter; it does not exclude other elements or steps. It is thus to be interpreted as specifying the presence of the stated features, integers, steps or components as referred to but does not preclude the presence or addition of one or more other features, integers, steps or components, or groups thereof. Thus, the scope of the expression “a device comprising means A and B” should not be limited to the devices consisting only of components A and B. It means that with respect to the disclosure, the only relevant components of the device are A and B.

The term “consisting of” refers to compositions, methods, and respective components thereof as described herein, which are exclusive of any element not recited in that description of the embodiment.

As used herein, the term “consisting essentially of” refers to those elements required for a given embodiment. The term permits the presence of additional elements that do not materially affect the basic and novel or functional characteristic(s) of that embodiment of the technology.

Reference throughout this specification to “one embodiment” or “an embodiment” means that a particular feature, structure or characteristic described in connection with the embodiment is included in at least one embodiment of the disclosure. Thus, appearances of the phrases “in one embodiment” or “in an embodiment” in various places throughout this specification are not necessarily all referring to the same embodiment but may. Furthermore, the particular features, structures or characteristics may be combined in any suitable manner, as would be apparent to one of ordinary skill in the art from this disclosure, in one or more embodiments.

Similarly, it should be appreciated that in the description of exemplary embodiments of the disclosure, various features of the disclosure are sometimes grouped together in a single embodiment, figure, or description thereof for the purpose of streamlining the disclosure and aiding the understanding of one or more of the various inventive aspects. This method of disclosure, however, is not to be interpreted as reflecting an intention that the claimed disclosure requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this disclosure.

Furthermore, while some embodiments described herein include some, but not other, features included in other embodiments, combinations of features of different embodiments are meant to be within the scope of the disclosure, and form different embodiments, as would be understood by those in the art. For example, in the following claims, any of the claimed embodiments can be used in any combination.

In the description provided herein, numerous specific details are set forth. However, it is understood that embodiments of the disclosure may be practiced without these specific details. In other instances, well-known methods, structures and techniques have not been shown in detail in order not to obscure an understanding of this description.

Other embodiments of the disclosure will be apparent to those skilled in the art from consideration of the specification and practice of the disclosure disclosed herein.

It is intended that the specification and examples be considered as exemplary only.

Each and every claim is incorporated into the specification as an embodiment of the disclosure. Thus, the claims are part of the description and are a further description and are in addition to the preferred embodiments of the disclosure.

Each of the claims set out a particular embodiment of the disclosure.

Definitions. The following terms are provided solely to aid in the understanding of the disclosure.

A plant pest concerns anything that has a negative impact on a plant, including insects, fungi, bacteria, virus and parasites.

Lignin is a complex organic substance that acts like a glue, binding the cells, fibers, and vessels that make up plants. It is the second most abundant biopolymer on earth, after cellulose, and plays a vital role in giving plants their strength and rigidity. Lignin is a complex aromatic polymer made up of various phenolic subunits that are mainly interlinked by ether and carbon-carbon bonds. Unlike cellulose, which is a carbohydrate, lignin is not easily broken down by microorganisms.

Lignin depolymerization is a process that breaks down lignin, a complex molecule found in plants, into smaller, components. In this particular disclosure, lignin depolymerization refers specifically to the breaking of ether bonds, and in particular Beta-O-4 ether bonds, that occur in native lignin yielding a liquid lignin oil comprising of components like phenolic monomers, dimers and trimers or specific oligophenolics with a DP from 1 to 8. This can be fractionated in fractions with selected DP.

Thermosolvolytic depolymerization of lignin (FIG. 1B) is a biorefinery method that combines lignocellulose biomass fractionation (solvent, heat and pressure disrupting the structure of the biomass and dissolves lignin and some of the hemicellulose) with lignin depolymerization under an inert gas or atmosphere. For instance, an inert gas or atmosphere selected from the group consisting of nitrogen, argon, helium and hydrogen.

As used herein, the terms “induce” or “inducing” refers to cause, or causing and to enhance, or enhancing and to boost or boosting and to activate or activating.

Described is a non-catalytic thermosolvolytic fractioning a depolymerization (FIG. 1B) process without the presence of a redox catalyst and under inert atmosphere. As we experienced the resulting lignin oils from such process, when not stored refrigerated, are prone to re-polymerization in an aqueous solution containing DMSO. In that case, the process of re-polymerization is suppressed or prevented by suppressors or stabilizing agents of the group consisting of 1,4-butanediol, 2-hydroxy-1-naphthoic acid, 2-naphthol, 2-naphthol-7-sulfonat, 2-naththol, 3-hydroxy-2-naphthoic acid, 4-hydroxybenzoic acid, 6-hydroxy-2-naphthoic acid, ascorbic acid, bovine serum albumin, citric acid, salicylic acid, cysteine, dimethylphloroglucinol, histidine, mannitol, o-dihydroxybenzene, p-benzenediol, phloroglucinol, resorcinol, soy protein isolate, syringic acid, and vanillic acid.

Reductive catalytic fractionation (RCF) (FIG. 1A) of lignocellulose is a biorefinery method that combines biomass fractionation (solvent, heat and pressure disrupting the structure of the biomass and dissolves lignin and some of the hemicellulose) with lignin depolymerization enabled by heterogeneous catalysis, more particularly a redox-active catalyst in a reductive environment (e.g., with the addition of hydrogen or H-donating agents). The output is carbohydrate enriched pulp (mainly cellulose) and a lignin oil comprising fractions of low molecular weight oligomeric and monomeric units of aromatic compounds which molecular weight fractions can be separated. RCF is a solvolytic process. As RCF typically uses a solvent system (e.g., methanol, ethanol, or water) to facilitate the solvolytic depolymerization of lignin from the lignocellulosic matrix. In addition, as the solvent plays a dual role by dissolving lignin fragments and stabilizing them against recondensation. Catalysts (often heterogeneous, like supported Ru, Ni, or Pd) operate within the solvent medium, enabling selective hydrogenolysis and hydrogenation reactions to depolymerize and stabilize lignin-derived fragments. The catalysts (often heterogeneous, like supported Ru, Ni, or Pd) operate within the solvent medium, enabling selective hydrogenolysis and hydrogenation reactions to depolymerize and stabilize lignin-derived fragments. Molecular hydrogen (H₂) or hydrogen donors (e.g., alcohol solvents like ethanol or formic acid) in solvolysis are commonly used to create a reductive environment, further facilitating solvolytic depolymerization. The solvent helps target lignin selectively, leaving cellulose and much of the hemicellulose largely unaffected in the solid phase (selective depolymerization), consistent with solvolytic principles. However, RCF in this application is also meant to comprise the reductive catalytic depolymerization of lignin solved in a suitable solvent (solvolytic catalytic depolymerization). Without post-treatment the pH of RCF liquids containing depolymerized lignin generally range from 4 to 6, reflecting the presence of phenolic and carboxylic acids and with hydrogenation stabilization this can shift to a neutral range (pH 6-7), as acidic groups are reduced or neutralized. When using ethanol/water systems with Ru/C catalysts, the pH after RCF typically stabilizes around 5-6 due to phenolic compounds. While employing stronger hydrogen donors or additional base (e.g., NaOH) to assist depolymerization can push the pH closer to neutral. We found that catalysis overcomes the tendency of lignin oils or lignin oil fractions to repolymerize. This can occur for lignin oils obtained from thermosolvolytic methods in absence of hydrogenation catalysis.

A suitable solvent for lignin depolymerization via RCF or non-catalytic thermosolvolytic fractioning the process is an aliphatic alcohol such as methanol, ethanol, 1-propanol, 1-butanol, 1-pentanol, 2-pentanol, 2-butanol, 2-pentanol, 3-pentanol, 2-methyl-1-propanol, 2-methyl-1-butanol, or 3-methyl-1-butanol or binary mixture with water thereof. Preferably the solvent of the group consisting of ethanol, methanol, butanol, acetone, ethyl acetate, toluene, tetrahydrofuran (THF), tetrahydrofuran (THF), gamma-valerolactone (GVL), and ionic liquid.

Micromixing means that the features of mixing are achieved at the molecular scale.

A polar aprotic solvent is one that has typically a dipole moment in the range of 2-5 D, a high dielectric constant of typically >15 and no hydrogen bond donors (i.e., they lack N—H or O—H groups). Suitable polar aprotic solvents for the plant defense elicitor formulations of the disclosure are, e.g., δ-valerolactone (DVL), cyrene, (dihydrolevoglucosenone), ethyl acetate, methyl lactate, acetone (dimethyl ketone) and limonene oxide).

PG is the abbreviation for the identified monomer, 4-propyl guaiacol.

PS is the abbreviation for the identified monomer, 4-propyl syringol.

DP is the abbreviation for degree of polymerization referring to the number of aromatic groups in the lignin oligomer.

Pulping in terms of RCF or non-catalytic thermosolvolytic fractioning is the separation cellulose fibers from the lignin or the other components, in generally via the organic solvents and sometimes water.

Fractionation in terms is the process of separating a mixture into its constituent parts based on their different properties. Fractionation allows processors to further separate the depolymerized lignin or lignin oils into fractions with different compositions, molecular weights or molecular sizes. In RCF, liquid-liquid extraction (LLE) is often used for separating lignin-derived phenolic monomers, dimers, and oligomers from the reaction mixture after depolymerization.

ISR is an abbreviation for induced systemic resistance. ISR is a defense mechanism in plants involving activation of the plant's immune response systemically (throughout the entire plant) after exposure to certain beneficial elicitors.

A “disease index” is a numerical representations of disease severity or incidence and it can quantify the impact of a disease on a plant population. The effect of ISR on disease suppression can be reflected indirectly and for evaluating the effectiveness of ISR-inducing treatments, researchers measure disease severity, pathogen growth, or symptom development, measurements that contribute to disease indices. A lower disease index in ISR-treated plants indicates successful suppression of diseases due to induced resistance.

A “disease index” in the context of A. thaliana is a quantitative measure used to evaluate the resistance of A. thaliana accessions to specific pathogens. This index is particularly useful in studying the genetic basis of disease resistance in plants. The disease index is calculated based on the number of necrotic lesions (areas of plant tissue that have died) that develop on the leaves of A. thaliana after inoculation with a pathogen.

The “disease index” for H. arabidopsidis in A. thaliana is a measure used to quantify the severity of infection by this oomycete pathogen. This index is based on the number of sporangiophores (structures that bear spores) observed on the plants after inoculation. The disease severity is scored by determining the number of sporangiophores per plant, with more sporangiophores indicating a higher level of infection or disease severity.

As used herein, the term “biomass” is used for the term “lignocellulosic material” and lignocellulosic material may be in the meaning of lignocellulose or material comprising lignocellulose.

The term “dry” or “dried” referring to a compound, component or composition means that the water content has been significantly reduced from the original form thereof. This is typically achieved through processes like dehydration, which remove water from the compound, component or composition by evaporation or other methods. The amount of moisture left in a dry compound, component or composition powder can vary depending on the specific type of compound, component or composition and the drying method used. It has to be interpreted to have a moist content under 12%, preferably under 10% and 7% and even having a moisture content of around 5% or even having have a moisture content of around 3%.

The term “elicitor” or “plant defense elicitor” as used herein refers to an exogenous defense-triggering molecule, e.g., inducer of the plant immune system or elicitors of natural plant defenses against pests (pathogens), abiotic and biotic stressors. When plants are attacked by pest such as pathogens, they defend themselves with an arsenal of mechanisms directed to fight infection or make the plant less attractive to that pest. As in most cellular responses to the environment, defense mechanisms can be activated when receptors directly or indirectly come in contact with pathogens. The ligands of these plant receptors are elicitors of the plant immune system. There is a wide variety of elicitors, including so-called non-specific elicitors or PAMPs (pathogen associated molecular patterns), e.g., degradation products of cell wall components of pathogens or derived from a plant cell wall, and pathogen-specific elicitors or effectors, e.g., avirulence gene products of pathogens such as AVR, such as AVR proteins, which play a crucial role in the gene-for-gene interactions between plants and pathogens and which avirulence gene products are recognized by plant resistance (R) genes, triggering defense responses in the host plant. Elicitors of the plant immune system comprise proteins, oligosaccharides, polysaccharides, lipids, glycolipids, glycoproteins, peptides of diverse origin, lipopeptides, algal extracts, extracts from the walls of plant material and/or fungal material, fungi, bacterial material and viral material, or yeast material and/or extracts. Elicitors also comprise salicylic acid, jasmonic acid, lipid peroxidation products and/or one or more of their esters. Elicitors of the plant immune system against insects play a crucial role in activating defense mechanisms to protect plants from herbivory. One such key elicitor is the jasmonate pathway, which is central in promoting resistance to a broad spectrum of insects. This pathway involves signal transduction pathways that include calcium ion fluxes, phosphorylation cascades, and the production of jasmonates, which are essential for plant defense against insect herbivores. Such a plant defense elicitor triggers the activation of defense responses, such as the biosynthesis of jasmonic acid, to defend against insect attacks.

Some exogenous defense-triggering molecules (plant defense elicitors) can induce a plant's defense system associated with extensive transcriptional and metabolic reprogramming of the genome. Elicitation of plants with elicitor molecules can result in the activation of a series of defense responses, including cell wall reinforcement by deposition of lignin and induction of an array of defense enzymes. Diverse plant defense responses induced by elicitors involve de novo synthesis and accumulation of antimicrobial phytoalexins, induction of cell death (hypersensitive response), production of activated oxygen species (oxidative burst), and modification of plant cell walls by deposition of callose (Bektas, 2022; Sudhamoy et al. 2010; Wang et al. 2004).

Elicitors can induce a range of responses in plants, including the production of antimicrobial compounds, cell death (hypersensitive response), generation of reactive oxygen species, and modification of cell walls. These responses contribute to enhancing plant resistance to pathogens and herbivores (Wang et al. 2004). The effects of elicitors on plants can vary depending on factors such as plant genotype, developmental stage, and environmental conditions (Chalal et al. 2015). Additionally, the application of elicitors can lead to the enhanced production of secondary metabolites in plants, which are crucial for defense against biotic stresses (Hatami et al. 2019).

Elicitors might be specifically recognized by the plant and subsequently induce defense responses against pathogens or herbivores in the attacked host (Maffei et al. 2012). Elicitor recognition by the plant is assumed to be mediated by specific receptors in the plant cell, localized either on the cell surface for some fungal elicitors or within the cell for certain bacterial elicitors, which initiate signaling processes that activate plant defenses (Angelova et al. 2006; Shinya et al. 2006).

Some plant defense elicitors have been recently discovered and tested. For instance, silicon has also been identified as a key element that can upregulate plant defense pathways against insects, providing physical and biochemical defense mechanisms (Alhousari and Greger, Plants 2018, 7(2), 33). Plant cells also recognize chitin fragments for defense signaling through a plasma membrane receptor. Chitin oligosaccharides are a representative general plant defense elicitor-inducing defense responses in a wide range of plant cells (Kaku et al. (2006) 11086-11091 PNAS Jul. 18, 2006 vol. 103 no. 29). Laminarin, a β-1,3 glucan oligosaccharide, oligo-galacturonan and sodium alginate are also plant defense elicitors and also found to be a plant defense elicitor (Priya Dey et al. PLoS ONE 14(9) (2019); Chalal et al. Front. Plant Sci., 19 May 2015 Sec. Plant Pathogen Interactions Vol. 6-2015 (2015) and Bektas (Horticulturae 2022, 8, 484). Cellodextrins act as elicitors of plant defense (Aziz et al. 2007 J. Exp. Bot. 58, 1463-1472; Souza et al. 2017 Plant Physiol. 173, 2383-2398; Johnson et al. 2018 Plant Physiol. 176, 2496-2514). In particular C1-oxidized (aldonic) cellodextrins, oligosaccharides composed of glucose units linked by β-1,4-glycosidic bonds, and the C4-oxidized cellodextrins, with the same basic structure as cellodextrins (glucose chains linked together β-1,4 glycosidic bonds) but an additional oxygen atom (an oxygen-containing functional groups, such as hydroxyl (—OH) or carboxyl (—COOH) group) bonded to the fourth carbon (C4) of the glucose unit at the non-reducing end are both known improve solubility and reactivity. With their modified structure containing oxygen-containing functional groups at the C1 and C4 positions, can act as molecular signals that plants recognize as indicators of potential danger. When plants perceive these signals, they may initiate defense responses, such as the production of defense-related proteins, activation of signaling pathways, or synthesis of secondary metabolites with antimicrobial properties (Josman Velasco et al. ACS Sustainable Chem. Eng. 2022,10, 16969-16984).

As used herein, the term “oligo-galacturonan” encompasses a chain of o-(1-4)-linked D-galacturonic acids. Oligo-galacturonans are derived from pectin, which is a major constituent of plant cell walls. Pectin consists of a complex set of polysaccharides, including homogalacturonans, which are linear chains of a-(1-4)-linked D-galacturonic acids. Oligo-galacturonans are released from these galacturonans through the action of pectolytic enzymes. Oligo-galacturonans particularly suitable as plant defense elicitor have a degree of polymerization higher than 8, preferably comprised between 9 and 20 or between 9 and 15.

The terms “chito-oligosaccharide” and “chitosan oligosaccharide” are used interchangeably herein and refer to a linear oligosaccharide composed of randomly distributed -(1-4)-linked D-glucosamine (deacetylated unit) and N-acetyl-D-glucosamine (acetylated unit). Chitosan is naturally found in few organisms, but is mostly produced industrially by deacetylation of chitin, which is the structural element in the exoskeleton of crustaceans (crabs, shrimp, etc.), insects, and in the cell walls of some fungi and other organisms. Chito-oligosaccharides used particularly suitable as plant defense elicitor have a degree of acetylation lower than 50%, lower than 40%, or lower than 30%, preferably about 25% and a degree of polymerization higher than 5, preferably comprised between 5 and 10.

Salts can be added to the composition hereof to ensure good ionic conditions and sucrose can be added to the composition hereof Preferably, sucrose is added in a concentration of from about 1 mM to 20 mM, most preferably about 5 to 10 mM sucrose. Sucrose triggers signaling through hexokinase and is also a wetting agent. Sucrose can act as wetting agents improve the spreading and sticking of water-based sprays on plant surfaces. This is crucial for applying pesticides, herbicides, and fungicides effectively. And hexokinase, the enzyme sucrose binds to, acts as a sensor, a binding that can activate genes involved in defense against pathogens or insects.

The term “fungicide” encompasses chemical or biological substances or compositions used to kill or inhibit fungi or oomycetes, e.g., by preventing sporulation, or their spores. Fungicides can exert their biological effect by different modes of action, for example, but not limited to, by interference with nucleic acid synthesis, mitosis and cell division, respiration, amino acids and protein synthesis, signal transduction, lipids and membrane synthesis, sterol biosynthesis, glucan synthesis in the pathogen or by inducing host plant defense.

Any fungicide can be included in the composition of the disclosure, such as, for example, a fungicide selected from: acylalanines (benalaxyl), anilinopyrimidines (cyprodinil or pyrimethanil), benzamides (fluopicolide or zoxamide), benzimidazoles (fuberidazole, thiabendazole or metrafenone), benzothiadiazoles (acibenzolar-S-methyl), carbamates (benthiavalicarb, iprovalicarb or propamocarb), carboxamides (boscalid), chloronitriles (chlorothalonil), chlorophenyls (tolclophos-methyl), cyanoacetamide oximes (cymoxanil), cyanoimidazoles (cyazofamid), dicarboximides (iprodione), dithiocarbamates (thiram, metiram, mancozeb, manebe or propineb), guanidines (dodine), hydroxyanilides (fenhexamid), imidazoles (fenamidone, imazalil or triflumizole), morpholines (dimethomorph, fenpropimorph, spiroxamine or dodemorph), phosphonates (fosetyl), oxathiins (flutolanil), oxazoles (famoxadone or hymexazol), phenylamides (metalaxyl or metalaxy-M), phenylpyridinamides (fluazinam), phenylpyrroles (fludioxonil), phthalimides (captan or folpet), quinazolinones (proquinazide), quinolins (quinoxyfen), strobilurins (dimoxystrobin, fluoxastrobin, kresomin-methyl, pyraclostrobin, trifloxystrobin or picoxystrobin), thiophenes (silthiofam), triazoles (difenoconazole, epoxyconazole, fenbuconazole, flusilazole, metconazole, myclobutanil, penconazole, propiconazole, tebuconazole, tetraconazole, triadimenol, triticonazole orprothioconazole), copper derivates (copper oxychloride, copper hydrochloride, copper oxide or copper sulphate) and sulfur. Preferably, the fungicide is chosen from the list comprising: phosphonates, benzamides, carbamates, dithiocarbamates, phthalimides, triazoles, quinolines, sulfur and cyanoimidazoles.

Phosphonates: The mode of action of the phosphonates is largely unknown but could involve inhibition of mitochondrial ATP synthase. Suitable examples of phosphonates are phosphorous acid derivatives, including phosphorous acid itself and its alkali metal or alkaline-earth metal salts. In a preferred embodiment the fungicides are ethyl hydrogenphosphonates such as fosetyl-AI, fosetyl-K and fosetyl-Na. Mention can be made of the phosphonates sold under the trade names Aliette™, Autograph™, Avalon™, Flanker™, Legion™, Linebacker™, Novasource™ Prodigy Signature™ and Quali-Pro™, which all comprise fosetyl-AI as active ingredient, and Magellan™ and Phostrol™, which comprise phosphorous acid as active ingredient.

Benzamides interfere with mitosis and cell division. In a preferred embodiment, the benzamides used in the composition of the disclosure contain 2,6-dichloro-N-[3-chloro-5-(trifluoromethyl)-2-pyridinyl]benzamide (fluopicolide) as active ingredient. Mention can be made of Infinito™.

Carbamates act by interfering with lipids and membrane synthesis. In a preferred embodiment, the carbamates used in the composition of the disclosure contain propamocarb, preferably propamocarb hydrochloride (propyl[3-(dimethylamino)propyl]carbamate hydrochloride) as the active ingredient. Mention can be made of the carbamates sold under the trade names Infinito™ and Stellar™ (comprising fluopicolide and propamocarb hydrochloride), Banol™ Previcur™, Proplant™ (comprising propamocarb hydrochloride) and Previcur™ Energy (comprising propamocarb and fosetyl-AI). Dithiocarbamates show multi-site contact activity. In a preferred embodiment, dithiocarbamates containing manganese ethylenebis(dithiocarbamate) (polymeric) complex with zinc salt (mancozeb) as active ingredient, are used in the composition of the disclosure. Mention can be made of the dithiocarbamates sold under the trade names Acrobat MZ™, CleviS™, Cuprofix MZ™, Dithane™, Evolve™, Fore™, Gaucho™ Gavel™, Junction™ Mancozide™, Manhandle™, Manzate™, Maxim™, Moncoat™, Nubark™, Penncozeb™ Pentathlon™, Potato Seed Treater™, Protect™, Ridomil Gold MZ™, SA-50™, Stature™, Tops MZ™, Wingman™ and Zyban™. Phthalimides also show multi-site contact activity. In a preferred embodiment, the phthalimides used in the plant defense elicitor composition of the disclosure comprise A/-(trichloromethylthio)phthalimide or 2-[(trichloromethyl)thio]-1-isoindole-1,3(2H)-dione (Folpet™) as active ingredient. Mention can be made of the phthalimides sold under the trade names Folpet™ and Fungitrol™.

Triazoles act by interfering with sterol biosynthesis in membranes. In a preferred embodiment, the triazoles used in the composition of the disclosure contain (2RS,3RS)-3-(2-chloorfenyl)-2-(4-fluorfenyl)-[(1H-1,2,4-triazool-1-yl)methyl]oxiraan (epoxyconazole) as active ingredient. Mention can be made of the triazole fungicide sold under the trade name Opus™.

Cyanoimidazoles act by interfering with the electron transport chain at the level of complex III in the inner membrane of mitochondria, which blocks oxidative phosphorylation powered by electron transfer. In a preferred embodiment, the cyanoimidazoles used in the composition of the disclosure contain 4-chloro-2-cyano-/V,/ /-dimethyl-5-(4-methylphenyl)-1-imidazole-1-sulfonamide (cyazofamid) as active ingredient. Mention can be made of the cyanoimidazole sold under the trade name Ranman™.

Quinolines act by interfering with, e.g., blocking, signal transduction. In a preferred embodiment, the quinolines used in the composition of the disclosure contain 5,7-dichloro-4-quinolyl 4-fluorophenyl ether (quinoxyfen) as active ingredient. Mention can be made of the quinoline fungicide sold under the trade names Legend™ or Quintec™.

Sulfur-containing fungicides show multi-site contact activity and contain sulfur as the active ingredient. Mention can be made of the sulfur-containing fungicide sold under the trade name THIOVIT®.

The compositions of the disclosure will typically contain additional components, known as co-formulants or adjuvants, to obtain a product with good handling, efficacy and stability properties. As used herein, the terms “co-formulant” or “adjuvant” designate any substance other than the main oligosaccharidic complex plant defense elicitor component defined herein, that is intentionally added to the plant defense elicitor composition of the disclosure.

In a certain embodiment, the composition hereof further comprises a co-formulant or adjuvant selected from the group comprising: surfactants, anti-freeze agents (including urea, ethylene glycol, propylene glycol or glycerol), preservative agents (including potassium sorbate, paraben and its derivates, 1,2-benzisothiazolin-3(2H)-one or essential oils), absorbent agents (including raids of corn or sawdust), thickeners (including clays or xanthan gum), buffers, sticker agents (including latex, silicon or alkoxylated alkyl), diluents (including rapeseed methyl ester) or any standard inert ingredient conventionally used in agricultural compositions, or a mixture thereof. In a particularly preferred embodiment, the composition hereof further comprises a surfactant.

With “surfactant” is meant herein a compound that lowers the surface tension of a liquid, allowing easier spreading. The surfactant can be a detergent, an emulsifier (including alkyl polyglucosides glycerol ester or polyoxyethylene (20) sorbitan monolaurate), or natural plant lecithin, or a biosurfactant, or a dispersing agent (including sodium chloride, potassium chloride, potassium nitrate, calcium chloride or starch of corn), a foaming agent (including derivates of tartric acid, malic acid or alcohols), a penetration enhancer, a humectant (including ammonium sulfate, glycerin or urea) or a wetting agent of ionic or non-ionic type or a mixture of such surfactants. The surfactants used in the disclosure are penetration enhancers, dispersing agents or emulsifiers.

The term “penetration enhancer” is understood herein as a compound that accelerates the uptake of active ingredient through the cuticle of a plant into the plant, i.e. the rate of uptake, and/or increases the amount of active ingredient absorbed into the plant. Classes of substances known as penetration enhancers, include alkyl phosphates, such as tributyl phosphate and tripropyl phosphate, and naphthalenesulphonic acid salts. Mention may be made, for example, of surfactants sold under the trade name DEHSCOFIX®, comprising castor oil and ethoxylated fatty acids, such as DEHSCOFIX CO 95® (available from Huntsman, USA), comprising C18 ethoxylated fatty acids from castor oil.

With “dispersing agent” is meant a substance added to a suspension, usually a colloid, to improve the separation of particles and to prevent settling or clumping. Mention can be made of the dispersing agent which is sold under the trade name Tensiofix Dp400™ (available from Ajinomoto OmniChem), essentially comprising organic sulfonate and 2-methylpentane-2,4-diol.

The term “abiotic” stress is used herein to refer to non-living chemical and/or physical factors in the environment that affect plant growth and/or development. Examples include extreme temperatures (heat or cold), water availability (e.g., drought), salinity (e.g., salt), and the like. Such abiotic factors are considered “stressors” when they influence the environment beyond its normal range of variation to adversely affect plant growth and/or development.

The “dry” state in the disclosure refers to a state that the water content is about 20 mass % or less and the water activity value is 0.85 or less. In addition, the water content is more preferably 15 mass % or less, more preferably 10 mass % or less, and more preferably 5 mass % or more. The lower limit is not particularly limited and is usually 0.1 mass % or more. Furthermore, the water activity value is preferably 0.80 or less and more preferably 0.75 or less.

The term “emulsifier” as used herein refers to a substance that stabilizes an emulsion, i.e. a mixture of two or more liquids. Mention can be made of the emulsifiers sold under the trade names TWEEN® 20, which essentially comprises polyoxyethylene (20) sorbitan monolaurate (polysorbate 20), and RADIA®, which essentially comprises alkyl polyglycosides. In a preferred embodiment, the surfactant comprises one or more of the following components: castor oil ethoxylate, rapeseed methyl ester, alkyl phosphates, tributyl phosphate, tripropyl phosphate, naphthalenesulphonic acid salts, organic sulfonate/2-methylpentane-2,4-diol, alkylpolyglucoside, siloxanes derivates, alkylsulfonates, polycarboxylates lignosulfonates, alkoxylated triglycerides, fatty amines polymers, dioctylsulfosuccinates or polyoxyethylene (20) sorbitan monolaurate (polysorbate 20), more preferably, the surfactant is C18-castor-oil-ethoxylate (DEHSCOFIX®), organic sulfonate/2-methylpentane-2,4-diol (Tensiofix™ Dp40) or polyoxyethylene (20) sorbitan monolaurate. The disclosure also discloses a composition comprising polar aprotic compounds with weak acidity like, for example, dimethyl sulfoxide (DMSO), enabling emulsification of the depolymerized lignin in water.

The term “biosurfactant” is understood a surface-active molecule produced by living organisms, typically microorganisms like bacteria or fungi. These special molecules can reduce surface tension and enhance the solubility and mobility of hydrophobic substances in aqueous environments and they have a unique structure with two key parts: a hydrophilic head, often composed of things like sugars, amino acids, or phosphate groups and a hydrophobic tail usually made of fatty acids or long chains of hydrocarbons. Biosurfactants are classified into different classes based on their chemical composition, including low molecular weight surface-active agents called biosurfactants and high molecular weight bioemulsifiers.

Any compound as used herein may be a part of a composition. The term “composition” generally refers to a thing composed of two or more components, and more specifically particularly denotes a mixture or a blend of two or more materials, such as elements, molecules, substances, biological molecules, or microbiological materials, as well as reaction products and decomposition products formed from the materials of the composition. By means of an example, a composition may comprise any compound as taught herein in combination with one or more other compounds or substances, be it one or more other compounds as taught herein or one or more other compounds or substances. For example, a composition may be obtained by combining, such as admixing, a compound as taught herein with the one or more other compounds or substances. For example, a composition may be obtained by decomposing a starting material, such as cellulose, into a mixture of a plurality of decomposition products.

In certain embodiments, the instant compositions may be configured as phytopharmaceutical or agrochemical compositions for treatment of a plant or plant protection composition. Phytopharmaceutical or agrochemical compositions typically comprise one or more active ingredients (chemically and/or biologically active materials having one or more beneficial effects on plant health) and one or more phytopharmaceutically acceptable carriers. As used herein, the active ingredient or one of the active ingredients is a plant defense elicitor. Compositions as typically used herein may be liquid, semisolid (e.g., gel), solid, or volatile or vapor-based, and may include solutions or dispersions, such as, for example, suspensions, emulsions, oil-in-water emulsions, water-in-oil emulsions, jellified aqueous solution or dispersion, solutions comprising a volatile organic solvent, etc. Examples of solid forms include, without limitation, powder, granules, pellets, water dispersible powder, water dispersible granules or water dispersible pellets. The composition may be formulated as a concentrate to be diluted before use, such as, for example, a soluble concentrate, an emulsifiable concentrate, a liquid concentrate and the like. Such composition may also be described as agrochemical composition. This a formulated mixture of chemical or biological substances designed for agricultural use. These compositions can include active ingredients, carriers, adjuvants, and other additives that enhance their efficacy, stability, or application properties. As used herein, the active ingredient or one of the active ingredients is a plant defense elicitor. Such compositions are used to protect plants from biotic stress such as pests and diseases or from abiotic stress.

As used herein, the term “carrier” broadly includes any and all solvents, diluents, bulking agents, buffers for pH control, dispersant, solubilizers, surfactants, wetting agents, emulsifiers, tackifiers, thickeners, binders, preservatives, antioxidants, cuticle solubilizing molecules, natural or regenerated mineral substances, and the like, and combinations thereof. Such materials should be non-toxic to the plants and should not interfere with the activity of the actives.

As used herein “solvolytic lignin depolymerization” means any one of the following processes reductive catalytic fractionation (RCF) of lignin or lignocellulose, non-catalytic thermosolvolytic depolymerization of lignin or lignocellulose or oxidative catalytic fractioning (OCF).

Typical salicylic acid pathway activators are salicylic acid (SA), benzothiadiazole (BTH), acibenzolar-S-methyl (ASM), chitosan, oligogalacturonides (OGs) and typical jasmonic acid pathway activators are jasmonic acid (JA), methyl jasmonate (MeJA), coronatine, hexanoic acid, and volicitin.

A preferred example of a plant treating phytopharmaceutically or agrochemically acceptable solvent is water, hence, compositions as taught herein may comprise water, i.e., may be aqueous solutions or dispersions. Further examples of suitable solvents include, but are not limited to, aromatic hydrocarbons, such as, for example, xylene mixtures or substituted naphthalenes; phthalates, such as, for example, dibutyl phthalate or dioctyl phthalate; aliphatic hydrocarbons, such as, for example, cyclohexane or paraffins; alcohols and glycols and their ethers and esters, such as, for example, ethanol, ethylene glycol, ethylene glycol mono methyl or monoethyl ether; ketones, such as, for example, cyclohexanone; strongly polar solvents, such as, for example, N-methyl-2-pyrrolidone, dimethyl sulfoxide or dimethylformamide; vegetable oils or epoxidized vegetable oils, such as, for example, epoxidized coconut oil or soybean oil; and water. In a particular aspect, the solvent is a volatile solvent, such as methanol and ethanol.

Non-limiting examples of solid carriers include, but are not limited to, natural mineral fillers, such as, for example, calcite, talcum, kaolin, montmorillonite or attapulgite; highly dispersed silicic acid or highly dispersed absorbent polymers; pumice, broken brick, sepiolite or bentonite; calcite or sand; dolomite or pulverized plant residues.

In certain embodiments, the compositions may comprise one or more surfactant, such as an anionic, non-ionic, amphoteric, or cationic surfactant, or a combination thereof, such as without limitation Triton X-100, non-ionic surfactant that has a hydrophilic polyethylene oxide chain (such as on average 9.5 ethylene oxide units) and an aromatic hydrocarbon hydrophobic group, 4-(1,1,3,3-tetramethylbutyl)-phenyl); a polysorbate-type non-ionic surfactant such as polyoxyethylene (20) sorbitan monolaurate, polyoxyethylene (20) sorbitan monopalmitate; and/or a non-ionic organosilicone surfactant such as SILWET® L-77 (3-(2-methoxyethoxy)propyl-methyl-bis(trimethylsilyloxy)silane).

In certain embodiments, the compositions may comprise one or more compounds miscible in organic solvents as well as water and having a weak acidity, such as without limitation dimethyl sulfoxide (DMSO) that enable the emulsification of lignin oil in water.

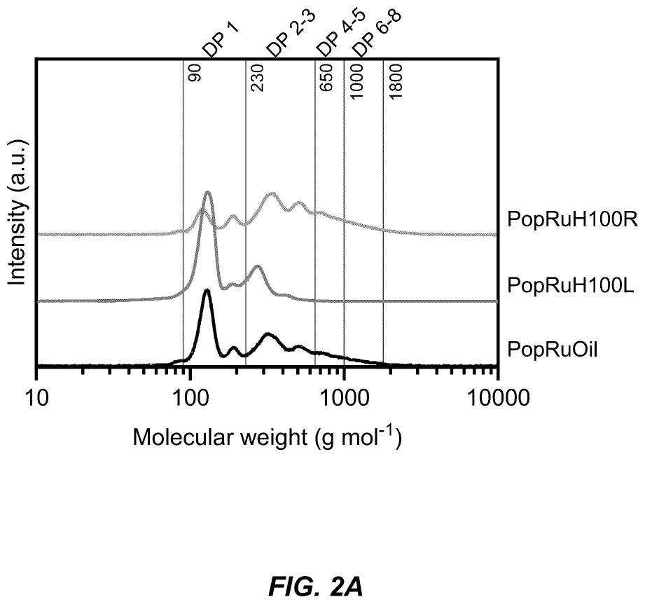

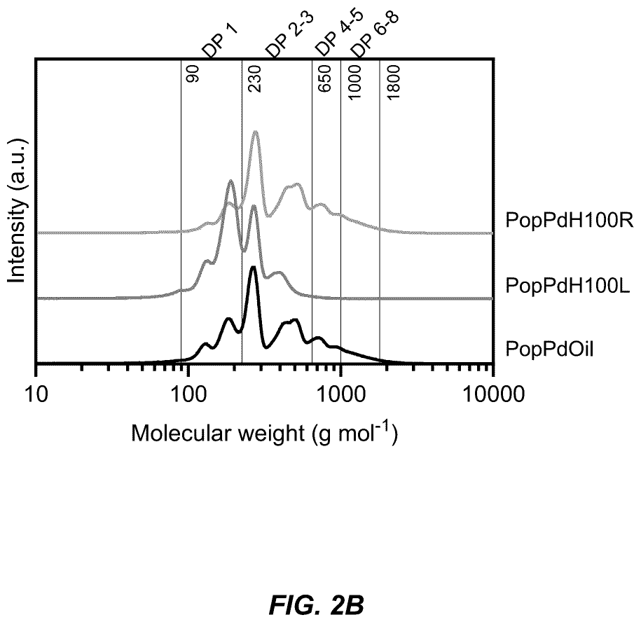

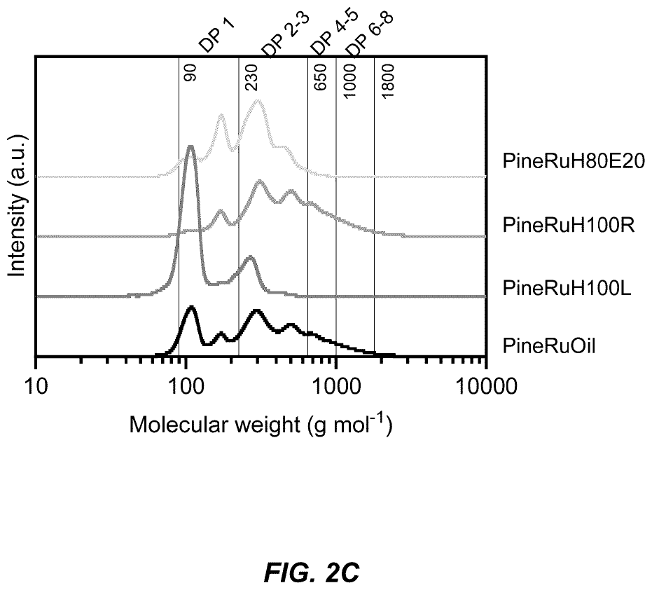

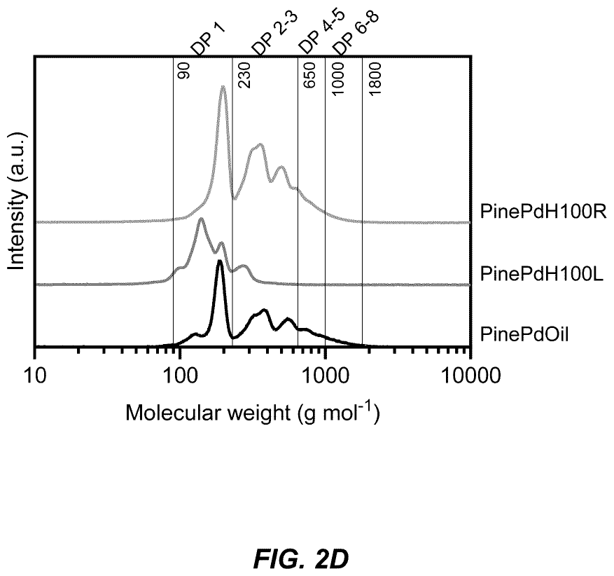

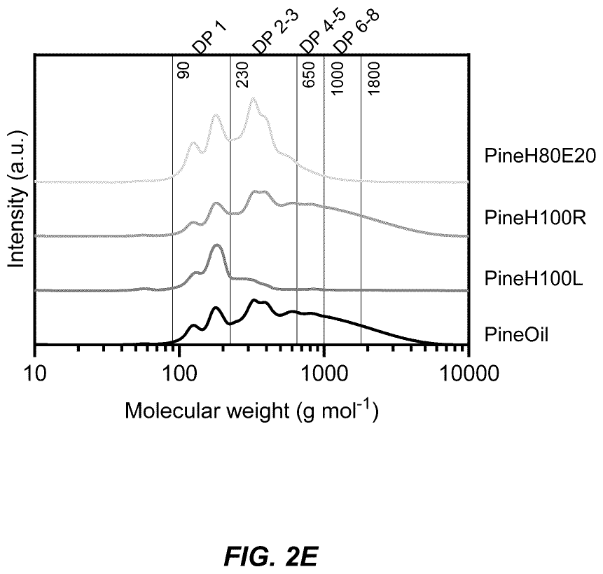

An embodiment of the disclosure is a) a plant defense elicitor characterized in that it comprises one or more a plant defense elicitor comprising, consisting of or essentially consisting of as active ingredient one or more aromatic compounds, with a DP of 2 to 8 (preferably 2-4) or one or more aromatic compounds, with a molecular mass 180 g/mol to 1000 g/mol, and yet more preferably between 230 g/mol to 650 g/mol (FIGS. 2A-2D), and/or wherein I) the aromatic compounds comprise at least one aromatic compound selected from the formulae

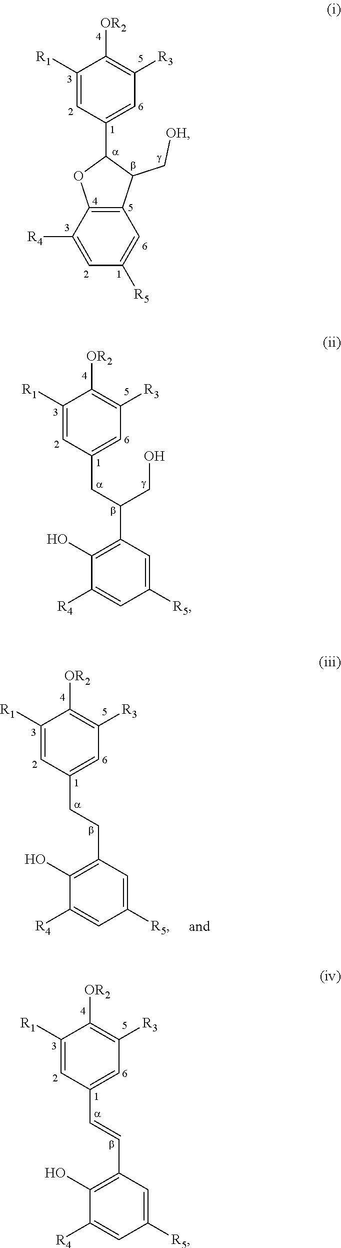

and
wherein each R₁, R₃, and R₄ is independently chosen from —H, —OH, O—CH3, a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or aromatic oligomer, or a carbon-oxygen linkage to an aromatic monomer or aromatic oligomer, and
wherein R₂ is —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer, or a carbon-oxygen linkage to an aromatic monomer or aromatic oligomer, and
wherein R₅ is selected from —H, a β-O-4 linkage to an aromatic monomer or aromatic oligomer, a β-5 linkage to an aromatic monomer or aromatic oligomer, a β-β linkage to an aromatic monomer or aromatic oligomer, a β-1 linkage to an aromatic monomer or aromatic oligomer, an end-unit selected from CH₃, —CH₂CH₃, —(CH₂)₂CH₃, —CH₂CH═CH₂, —CH═CHCH₃, —(CH₂)₂CH₂OH, —(CH₂)₂CHO, —CH═CHCH₂OH, —(CH₂)₂CH₂OCH₃, —CH═CHCH₂OCH₃, —(CH₂)₂CH₂OCH₂CH₃, —CH═CHCH₂OCH₂CH₃, —(CH₂)₂CH₂O(CH₂)₂CH₃, —CH═CHCH₂O(CH₂)₂CH₃, —(CH₂)₂CH₂OCH(CH₃)₂, —CH═CHCH₂OCH(CH₃)₂, —(CH₂)₂CH₂O(CH₂)₃CH₃, —CH═CHCH₂O(CH₂)₃CH₃, or a carbon linkage to an aromatic monomer or aromatic oligomer; and/or
the aromatic compounds comprise at least one aromatic compound selected from the formulae

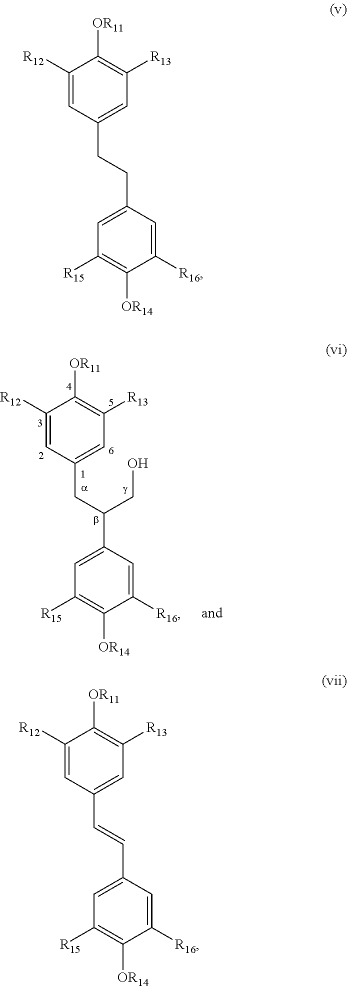

and
wherein each R₁₂, R₁₃, R₁₅ and R₁₆ is independently chosen from —H, —OH, O—CH₃, a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or aromatic oligomer, or a carbon-oxygen linkage to an aromatic monomer or aromatic oligomer,
wherein R₁₁ and R₁₄ is independently chosen from —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer or a carbon-oxygen linkage to an aromatic monomer or aromatic oligomer, and/or III) wherein at the aromatic compounds comprise at least one aromatic compound selected from the formula

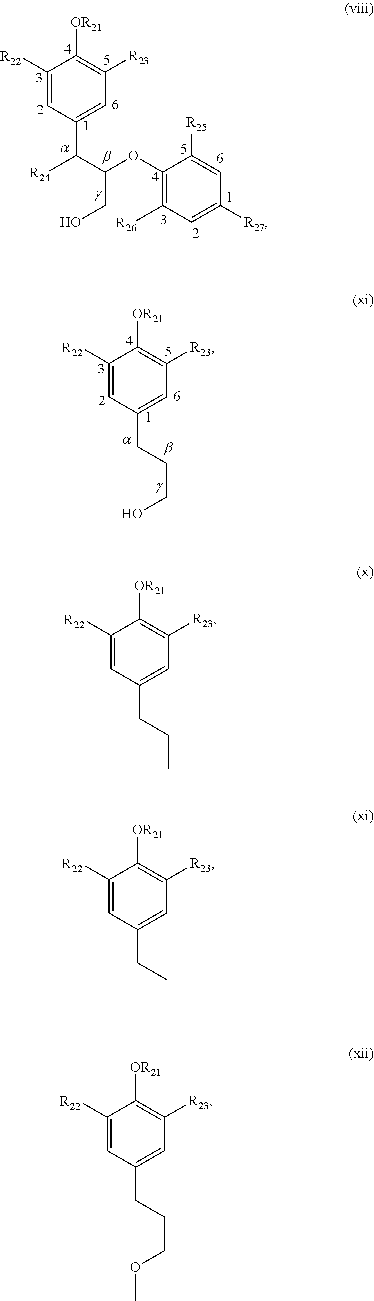

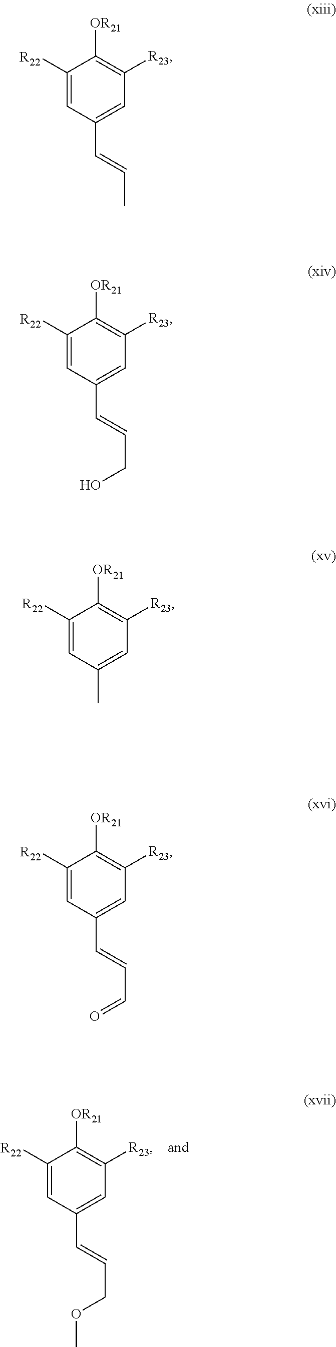

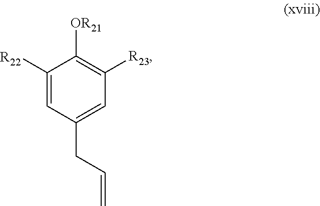

each with at least one linkage to an aromatic monomer or aromatic oligomer, and
wherein each R₂₂, R₂₃, R₂₅ and R₂₆ is independently chosen from —H, —OH, O—CH₃, a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or an aromatic oligomer, or a carbon-oxygen linkage to an aromatic monomer or aromatic oligomer,
wherein R₂₁ is independently chosen from —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer or a carbon-oxygen linkage to an aromatic monomer or aromatic oligomer,
wherein R₂₄ is independently chosen from —H, OH, or —O-Alkyl wherein the alkyl group is derived from the alcohol solvent of the process,
wherein R₂₇ is independently chosen from —H, a β-O-4 linkage to an aromatic monomer or aromatic oligomer, a β-5 linkage to an aromatic monomer or aromatic oligomer, a β-β linkage to an aromatic monomer or aromatic oligomer, a β-1 linkage to an aromatic monomer or aromatic oligomer, end-unit selected from CH₃, —CH₂CH₃, —(CH₂)₂CH₃, —CH₂CH═CH₂, —CH═CHCH₃, (CH₂)₂CH₂OH, —(CH₂)₂CHO, —CH═CHCH₂OH, —(CH₂)₂CH₂OCH₃, —CH═CHCH₂OCH₃, —(CH₂)₂CH₂OCH₂CH₃, —CH═CHCH₂OCH₂CH₃, —(CH₂)₂CH₂O(CH₂)₂CH₃, —CH═CHCH₂O(CH₂)₂CH₃, —(CH₂)₂CH₂OCH(CH₃)₂, —CH═CHCH₂OCH(CH₃)₂, —(CH₂)₂CH₂O(CH₂)₃CH₃, —CH═CHCH₂O(CH₂)₃CH₃, a carbon linkage to an aromatic monomer or an aromatic oligomer, and a co-formulant selected from the group comprising: surfactants, anti-freeze agents (including urea, ethylene glycol, propylene glycol or glycerol), preservative agents (including potassium sorbate, paraben and its derivates, 1,2-benzisothiazolin-3(2H)— at least one essential oil), absorbent agents (including raids of corn or sawdust), thickeners (including clays or xanthan gum), buffers, sticker agents (including latex, silicon or alkoxylated alkyl), diluents (including rapeseed methyl ester) or any standard inert ingredient conventionally used in agricultural compositions, or a mixture thereof, preferably, the co-formulant is a surfactant selected among a detergent, an emulsifier (including alkyl polyglucosides, glycerol ester or polyoxyethylene (20) sorbitan monolaurate (polysorbate 20)), a dispersing agent (including sodium chloride, potassium chloride, potassium nitrate, calcium chloride or starch of corn), a foaming agent (including derivates of tartric acid, malic acid or alcohols), a penetration enhancer, a humectant (including ammonium sulfate, glycerin or urea) or a wetting agent of ionic or non-ionic type or a mixture thereof, more preferably, the surfactant comprises one or more of the following components: castor oil ethoxylate, rapeseed methyl ester, alkyl phosphates, tributyl phosphate, tripropyl phosphate, naphthalenesulphonic acid salts, organic sulfonate/2-methylpentane-2,4-diol, alkylpolyglucoside, siloxanes derivates, alkylsulfonates, polycarboxylates, lignosulfonates, alkoxylated triglycerides, fatty amines polymers, dioctylsulfosuccinates or polyoxyethylene (20) sorbitan monolaurate, most preferably, the surfactant is C18-castor-oil-ethoxylate (DEHSCOFIX®), organic sulfonate/2-methylpentane-2,4-diol (Tensiofix Dp40™) or polyoxyethylene (20) sorbitan monolaurate. In another embodiment, the compositions of the disclosure also comprise one or more other active compounds selected from the group comprising: herbicides, insecticides, plant growth regulators or other plant immune system elicitors.

In a further embodiment, this composition of the disclosure can further comprise a further plant immune system elicitor chosen among silica, copper, sulfur, aluminum, vanadium, cobalt, nickel, iron, silver, salicylic acid and its derivates (including acetyl-salicylic acid, isonicotinic acid, acibenzolar-S-methyl), jasmonic acid and its derivates (including methyl jasmonate), ethylene and its derivates, polysaccharides (including glucans, xyloglucans, cellodextrins in particular with modified structure containing oxygen-containing functional groups at the C1 and C4 positions, chitin, chitosans, fucans, galactofucans, xylans, galactans, alginates, galacturonans, apiogalacturonans, fructans including inulin, mannans, xylomannans, galactomannans, glucomannans and galactomannans), algae extracts (green algae extracts including ulvans, brown algae extracts including laminarin, and red algae extracts including carragenans), oligosaccharides (including trehalose), peptides (including systemin, 13-pep, flg-22, glutathion), amino acids, proteins (including harpin and flagellin), peptone, beef extract, essential oils (including cumin, anise, mint, cinnamon, thyme, basil, cardamom, coriander, oregano, manzanilla, clove, jojoba and tea tree oils), lipids (including ergosterol, amphotericin, sphingolipids, cerebrosides), glycolipids (including syringolids), glycoproteins (including cryptogeins), lipopeptides, lipoproteins (including volicitin), yeast extracts (including extracts from Saccharomyces, Candida, Pichia, Aureobasidium and more particularly Saccharomyces cerevisiae, Candida famata, Candida oleophila, Pichia guilliermondii, Aureobasidium pullulans), fungal extracts (including extracts from Trichoderma, Megasperma, Pyricularia, Alternaria, Pythium, Puccinia, Colletotrichum, Verticillium, Magna porthe), bacterial extracts (including extracts from Escherichia, Rhyzobia, Pseudomonas), BABA, probenazole, isothianil, phosphorous acid and its derivates (including aluminum, sodium and potassium fosetyl), horsetail extracts, potassium iodide and potassium thiocyanate, citrus extracts, yucca extracts salix extracts and plant decoctions (including nettle decoction). Preferably, the further plant immune system elicitor contains laminarin (a linear β(1→3)-glucan with (1→6)-linkages) such as, for example, VACCIPLANT FRUIT®.

Yet another embodiment of disclosure is a) a plant defense elicitor characterized in that it comprises one or more a plant defense elicitor comprising, consisting of or essentially consisting of as active ingredient one or more aromatic compounds, with a DP of 2 to 8 (preferably 2-4) or one or more aromatic compounds, with a molecular mass 180 g/mol to 1000 g/mol (preferably between 230 g/mol to 650 g/mol) and wherein and/or I) the aromatic compounds comprise at least one aromatic compound selected from the formulae

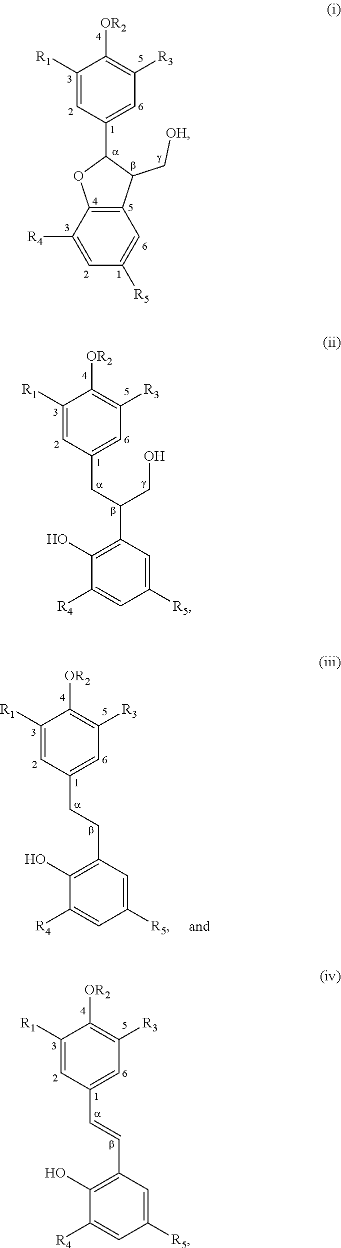

and
wherein each R₁, R₃, and R₄ is independently chosen from —H, —OH, O—CH₃, a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or aromatic oligomer, or a carbon-oxygen linkage to an aromatic monomer or aromatic oligomer, and
wherein R₂ is —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer, or a carbon-oxygen linkage to an aromatic monomer or aromatic oligomer, and
wherein R₅ is selected from —H, a β-O-4 linkage to an aromatic monomer or aromatic oligomer, a β-5 linkage to an aromatic monomer or aromatic oligomer, a β-β linkage to an aromatic monomer or aromatic oligomer, a β-1 linkage to an aromatic monomer or aromatic oligomer, an end-unit selected from CH₃, —CH₂CH₃, —(CH₂)₂CH₃, —CH₂CH═CH₂, —CH═CHCH₃, —(CH₂)₂CH₂OH, —(CH₂)₂CHO, —CH═CHCH₂OH, —(CH₂)₂CH₂OCH₃, —CH═CHCH₂OCH₃, —(CH₂)₂CH₂OCH₂CH₃, —CH═CHCH₂OCH₂CH₃, —(CH₂)₂CH₂O(CH₂)₂CH₃, —CH═CHCH₂O(CH₂)₂CH₃, —(CH₂)₂CH₂OCH(CH₃)₂, —CH═CHCH₂OCH(CH₃)₂, —(CH₂)₂CH₂O(CH₂)₃CH₃, —CH═CHCH₂O(CH₂)₃CH₃, or a carbon linkage to an aromatic monomer or aromatic oligomer; and/or
the aromatic compounds comprise at least one aromatic compound selected from the formulae

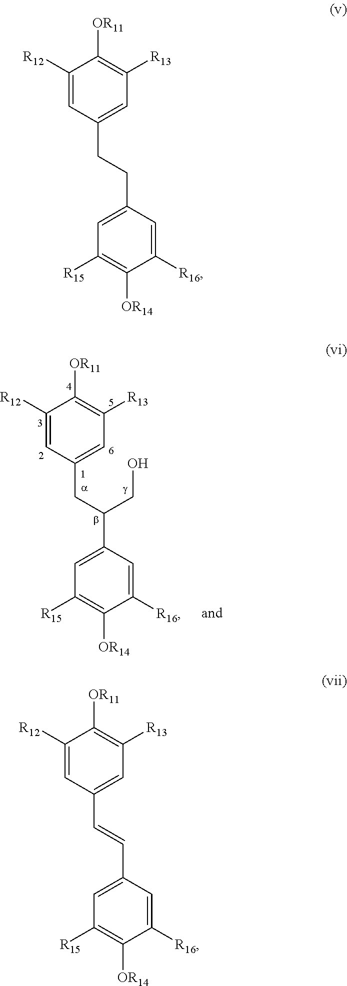

and
wherein each R₁₂, R₁₃, R₁₅ and R₁₆ is independently chosen from —H, —OH, O—CH₃, a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or aromatic oligomer, or a carbon-oxygen linkage to an aromatic monomer or aromatic oligomer,
wherein R₁₁ and R₁₄ is independently chosen from —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer or a carbon-oxygen linkage to an aromatic monomer or aromatic oligomer, and/or III) wherein at the aromatic compounds comprise at least one aromatic compound selected from the formula

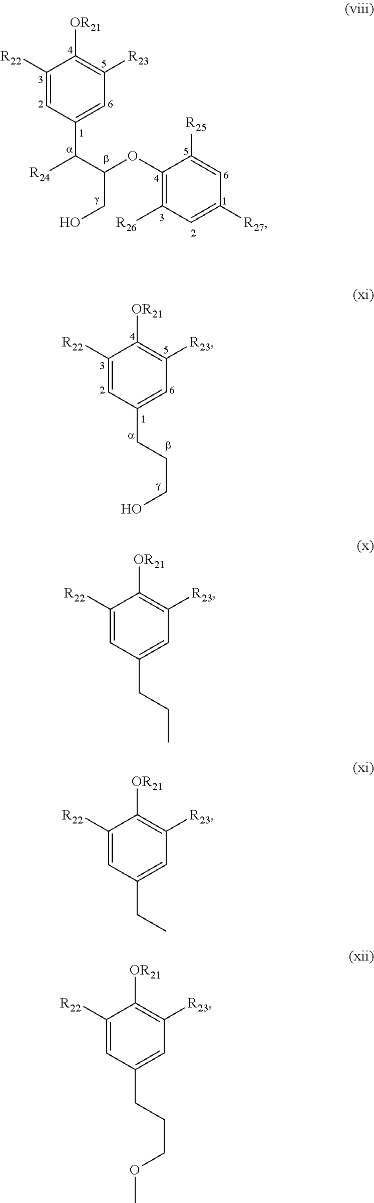

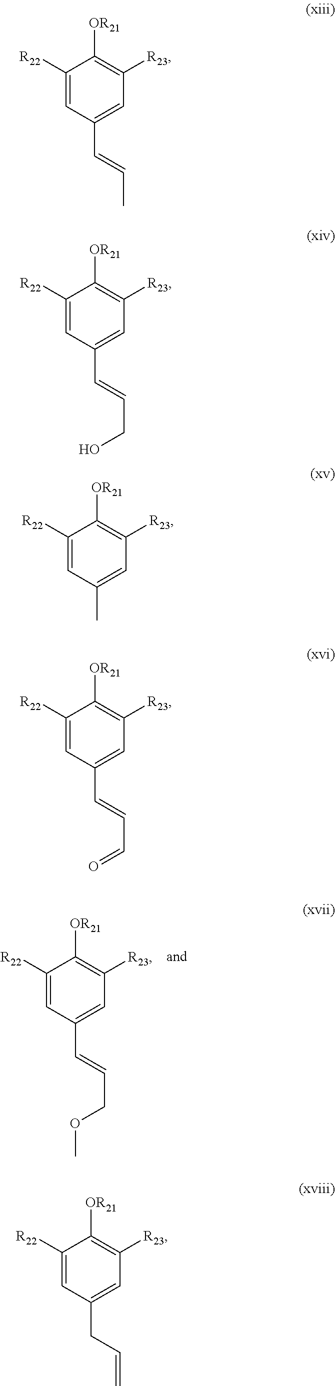

each with at least one linkage to an aromatic monomer or aromatic oligomer, and
wherein each R₂₂, R₂₃, R₂₅ and R₂₆ is independently chosen from —H, —OH, O—CH3, a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or an aromatic oligomer, or a carbon-oxygen linkage to an aromatic monomer or aromatic oligomer,
wherein R₂₁ is independently chosen from —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer or a carbon-oxygen linkage to an aromatic monomer or aromatic oligomer,
wherein R₂₄ is independently chosen from —H, OH, or —O-Alkyl wherein the alkyl group is derived from the alcohol solvent of the process,
wherein R₂₇ is independently chosen from —H, a β-O-4 linkage to an aromatic monomer or aromatic oligomer, a β-5 linkage to an aromatic monomer or aromatic oligomer, a 3-3 linkage to an aromatic monomer or aromatic oligomer, a β-1 linkage to an aromatic monomer or aromatic oligomer, end-unit selected from CH₃, —CH₂CH₃, —(CH₂)₂CH₃, —CH₂CH═CH₂, —CH═CHCH₃, (CH₂)₂CH₂OH, —(CH₂)₂CHO, —CH═CHCH₂OH, —(CH₂)₂CH₂OCH₃, —CH═CHCH₂OCH₃, —(CH₂)₂CH₂OCH₂CH₃, —CH═CHCH₂OCH₂CH₃, —(CH₂)₂CH₂O(CH₂)₂CH₃, —CH═CHCH₂O(CH₂)₂CH₃, —(CH₂)₂CH₂OCH(CH₃)₂, —CH═CHCH₂OCH(CH₃)₂, —(CH₂)₂CH₂O(CH₂)₃CH₃, —CH═CHCH₂O(CH₂)₃CH₃, a carbon linkage to an aromatic monomer or an aromatic oligomer, and a second plant defense elicitor, preferably a second plant defense elicitor chosen among silica, copper, sulfur, aluminum, vanadium, cobalt, nickel, iron, silver, salicylic acid and its derivates (including acetyl-salicylic acid, isonicotinic acid, acibenzolar-S-methyl), jasmonic acid and its derivates (including methyl jasmonate), ethylene and its derivates, polysaccharides (including glucans, xyloglucans, chitin, chitosans, fucans, galactofucans, xylans, galactans, alginates, galacturonans, apiogalacturonans, fructans including inulin, mannans, xylomannans, galactomannans, glucomannans and galactomannans), polyols, algae extracts (green algae extracts including ulvans, brown algae extracts including laminarin, and red algae extracts including carragenans), oligosaccharides (including trehalose), peptides (including systemin, 13-pep, flg-22, glutathion), amino acids, proteins (including harpin and flagellin), peptone, beef extract, essential oils (including cumin, anise, mint, cinnamon, thyme, basil, cardamom, coriander, oregano, manzanilla, clove, jojoba and tea tree oils), lipids (including ergosterol, amphotericin, sphingolipids, cerebrosides), glycolipids (including syringolids), glycoproteins (including cryptogeins), lipopeptides, lipoproteins (including volicitin), yeast extracts (including extracts from Saccharomyces, Candida, Pichia, Aureobasidium and more particularly S. cerevisiae, C. famata, C. oleophila, P. guilliermondii, A. pullulans), fungal extracts (including extracts from Trichoderma, Megasperma, Pyricularia, Alternaria, Pythium, Puccinia, Colletotrichum, Verticillium, Magnaporthe), bacterial extracts (including extracts from Escherichia, Rhyzobia, Pseudomonas), BABA, probenazole, isothianil, phosphorous acid and its derivates (including aluminum, sodium and potassium fosetyl), horsetail extracts, potassium iodide and potassium thiocyanate, citrus extracts, yucca extracts salix extracts and plant decoctions (including nettle decoction), more preferably, the second plant defense elicitor contains i-3(1-6)glucane (laminarin). In another preferred embodiment, the compositions of the disclosure further comprise a further plant immune system elicitor that contains silicon or silicium (Si), such as, for example, silica (SiO2) or silicates, including sodium silicate (Na₂SiO₃). Preferably, the further plant immune system elicitor is a silicate (e.g., sodium silicate.

The disclosure also discloses a composition comprising:
a plant defense elicitor characterized in that it comprises one or more a plant defense elicitor comprising, consisting of, or essentially consisting of as active ingredient one or more aromatic compounds, with a DP of 2 to 8 (preferably 2-4) or one or more aromatic compounds, with a molecular mass 180 g/mol to 1000 g/mol, and yet more preferably between 230 g/mol to 650 g/mol (FIGS. 2A-2D), and wherein and/or I) the aromatic compounds comprise at least one aromatic compound selected from the formulae

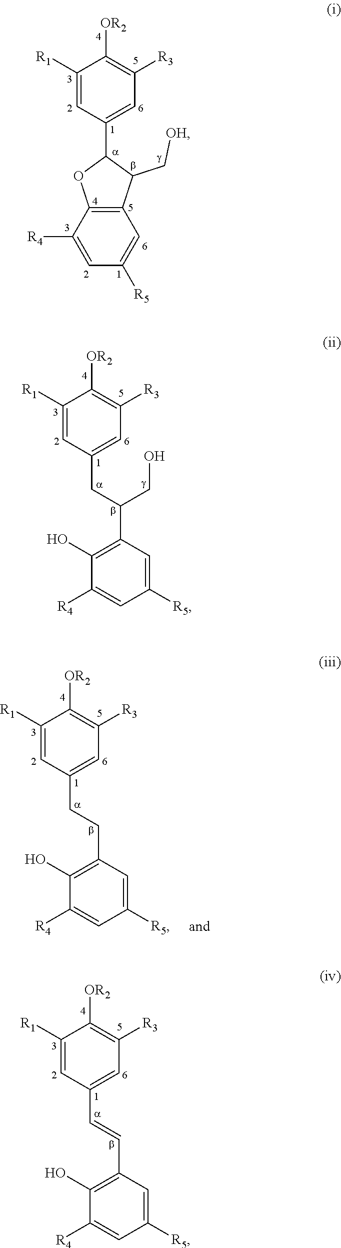

and
wherein each R₁, R₃, and R₄ is independently chosen from —H, —OH, O—CH3, a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or aromatic oligomer, or a carbon-oxygen linkage to an aromatic monomer or aromatic oligomer, and
wherein R₂ is —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer, or a carbon-oxygen linkage to an aromatic monomer or aromatic oligomer, and
wherein R₅ is selected from —H, a β-O-4 linkage to an aromatic monomer or aromatic oligomer, a β-5 linkage to an aromatic monomer or aromatic oligomer, a β-β linkage to an aromatic monomer or aromatic oligomer, a β-1 linkage to an aromatic monomer or aromatic oligomer, an end-unit selected from CH₃, —CH₂CH₃, —(CH₂)₂CH₃, —CH₂CH═CH₂, —CH═CHCH₃, —(CH₂)₂CH₂OH, —(CH₂)₂CHO, —CH═CHCH₂OH, —(CH₂)₂CH₂OCH₃, —CH═CHCH₂OCH₃, —(CH₂)₂CH₂OCH₂CH₃, —CH═CHCH₂OCH₂CH₃, —(CH₂)₂CH₂O(CH₂)₂CH₃, —CH═CHCH₂O(CH₂)₂CH₃, —(CH₂)₂CH₂OCH(CH₃)₂, —CH═CHCH₂OCH(CH₃)₂, —(CH₂)₂CH₂O(CH₂)₃CH₃, —CH═CHCH₂O(CH₂)₃CH₃, or a carbon linkage to an aromatic monomer or aromatic oligomer; and/or
the aromatic compounds comprise at least one aromatic compound selected from the formulae

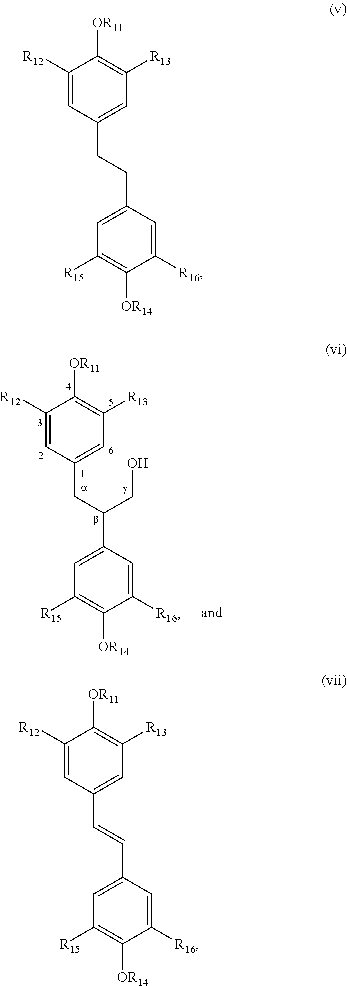

and
wherein each R₁₂, R₁₃, R₁₅ and R₁₆ is independently chosen from —H, —OH, O—CH₃, a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or aromatic oligomer, or a carbon-oxygen linkage to an aromatic monomer or aromatic oligomer,
wherein R₁₁ and R₁₄ is independently chosen from —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer or a carbon-oxygen linkage to an aromatic monomer or aromatic oligomer, and/or III) wherein at the aromatic compounds comprise at least one aromatic compound selected from the formula

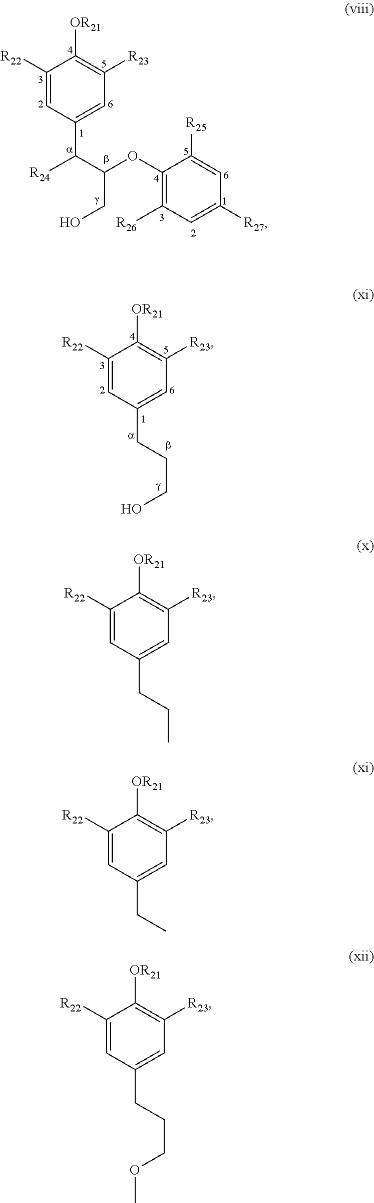

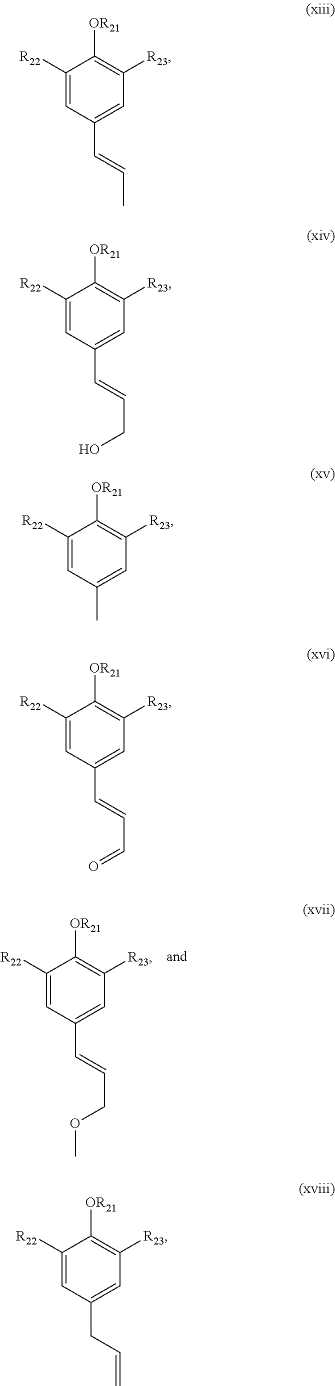

each with at least one linkage to an aromatic monomer or aromatic oligomer and
wherein each R₂₂, R₂₃, R₂₅ and R₂₆ is independently chosen from —H, —OH, O—CH₃, a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or an aromatic oligomer, or a carbon-oxygen linkage to an aromatic monomer or aromatic oligomer,
wherein R₂₁ is independently chosen from —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer or a carbon-oxygen linkage to an aromatic monomer or aromatic oligomer,
wherein R₂₄ is independently chosen from —H, OH, or —O-Alkyl wherein the alkyl group is derived from the alcohol solvent of the process,
wherein R₂₇ is independently chosen from —H, a β-O-4 linkage to an aromatic monomer or aromatic oligomer, a β-5 linkage to an aromatic monomer or aromatic oligomer, a β-β linkage to an aromatic monomer or aromatic oligomer, a β-1 linkage to an aromatic monomer or aromatic oligomer, end-unit selected from CH₃, —CH₂CH₃, —(CH₂)₂CH₃, —CH₂CH═CH₂, —CH═CHCH₃, (CH₂)₂CH₂OH, —(CH₂)₂CHO, —CH═CHCH₂OH, —(CH₂)₂CH₂OCH₃, —CH═CHCH₂OCH₃, —(CH₂)₂CH₂OCH₂CH₃, —CH═CHCH₂OCH₂CH₃, —(CH₂)₂CH₂O(CH₂)₂CH₃, —CH═CHCH₂O(CH₂)₂CH₃, —(CH₂)₂CH₂OCH(CH₃)₂, —CH═CHCH₂OCH(CH₃)₂, —(CH₂)₂CH₂O(CH₂)₃CH₃, —CH═CHCH₂O(CH₂)₃CH₃, a carbon linkage to an aromatic monomer or an aromatic oligomer, the first plant elicitor in proportions ranging from 1:50 to 50:1, preferably from 1:40 to 40:1, more preferably from 1:30 to 30:1, even more preferably from 1:20 to 20:1, most preferably from 1:10 to 10:1 and, e.g., is 1:9, 1:8, 1:7, 1:6, 1:5, 1:4, 1:3, 1:2, 1:1, 2:1, 3:1, 4:1, 5:1, 6:1, 7:1, 8:1 or 9:1, and
a second plant defense elicitor, preferably a second plant defense elicitor that contains silicon, more preferably, the second plant defense elicitor is a silicate, even more preferably, the second plant defense elicitor is sodium silicate. In a preferred embodiment, the compositions of the disclosure comprising a plant defense elicitor and a second plant defense elicitor further comprise an adjuvant, preferably a surfactant, more preferably a surfactant comprising polyoxyethylene (20) sorbitan monolaurate, a biosurfactant or a plant lecithin.

The compositions of the disclosure encompass not only compositions which are ready to be applied to the plant by means of a suitable device, such as a spraying device, but also the commercial concentrated compositions which have to be diluted before application to the plant.

Such compositions are themselves in quite diverse, solid or liquid forms. Solid composition forms include powders for dusting and granules, in particular, those obtained by extrusion, by compacting, by impregnation of a granulated support or by granulation from a powder, tablets or effervescent lozenges. Liquid composition forms or forms intended to constitute liquid compositions when applied include solutions, in particular, water-soluble concentrates, emulsions, concentrated suspensions, dispersions, aerosols and wettable granules and powders (or powders for spraying), pastes, gels and water-soluble packaging. In another aspect, the disclosure relates to the use of the compositions of the disclosure in agricultural applications, more particularly for protecting plants against (infection by) plant pathogens.

The disclosure not only provides for the simultaneous use of different components of the compositions, i.e., the use of the compositions, but also provides in the sequential use of the different components of the compositions. For instance, it is herein described that the sequential use of the plant defense elicitor from a lignin depolymerization process and fractioning in compositions comprising oligophenolics with a DP of 2 to 5, preferably of 2 to 3, from depolymerized lignin or decomposed lignin (FIGS. 2A-2D) or from structural identical oligophenolic and a fungicide also results in enhanced efficacy of the fungicide.

Further, it is herein described that the sequential use of the plant defense elicitor characterized in that it comprises one or more plant defense elicitors comprising, consisting of or essentially consisting of as active ingredient one or more aromatic compounds, with a DP of 2 to 8 (preferably 2-4) or one or more aromatic compounds, with a molecular mass 180 g/mol to 1000 g/mol (preferably between 230 to 650 g/mol) (FIGS. 2A-2D), and wherein and/or I) the aromatic I compounds comprise at least one aromatic compound selected from the formulae

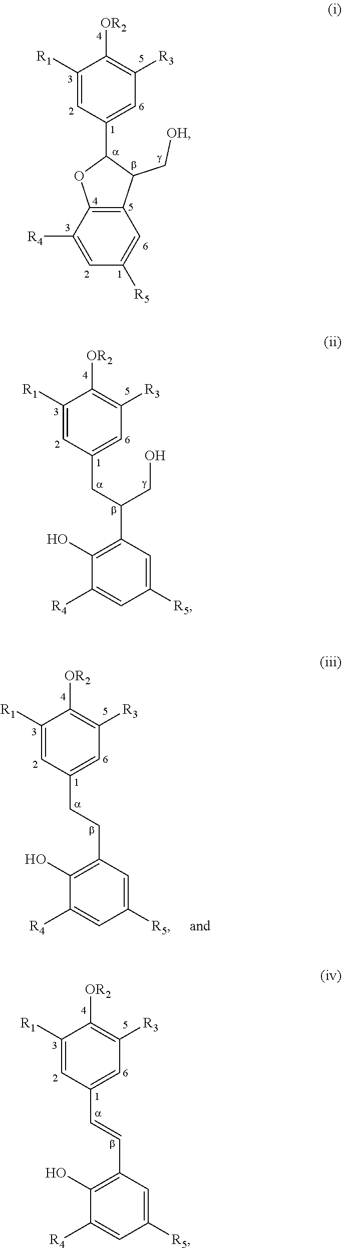

and
wherein each R₁, R₃, and R₄ is independently chosen from —H, —OH, O—CH3, a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or aromatic oligomer, or a carbon-oxygen linkage to an aromatic monomer or aromatic oligomer, and
wherein R₂ is —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer, or a carbon-oxygen linkage to an aromatic monomer or aromatic oligomer, and
wherein R₅ is selected from —H, a β-O-4 linkage to an aromatic monomer or aromatic oligomer, a β-5 linkage to an aromatic monomer or aromatic oligomer, a β-β linkage to an aromatic monomer or aromatic oligomer, a β-1 linkage to an aromatic monomer or aromatic oligomer, an end-unit selected from CH₃, —CH₂CH₃, —(CH₂)₂CH₃, —CH₂CH═CH₂, —CH═CHCH₃, —(CH₂)₂CH₂OH, —(CH₂)₂CHO, —CH═CHCH₂OH, —(CH₂)₂CH₂OCH₃, —CH═CHCH₂OCH₃, —(CH₂)₂CH₂OCH₂CH₃, —CH═CHCH₂OCH₂CH₃, —(CH₂)₂CH₂O(CH₂)₂CH₃, —CH═CHCH₂O(CH₂)₂CH₃, —(CH₂)₂CH₂OCH(CH₃)₂, —CH═CHCH₂OCH(CH₃)₂, —(CH₂)₂CH₂O(CH₂)₃CH₃, —CH═CHCH₂O(CH₂)₃CH₃, or a carbon linkage to an aromatic monomer or aromatic oligomer; and/or
the aromatic compounds comprise at least one aromatic compound selected from the formulae

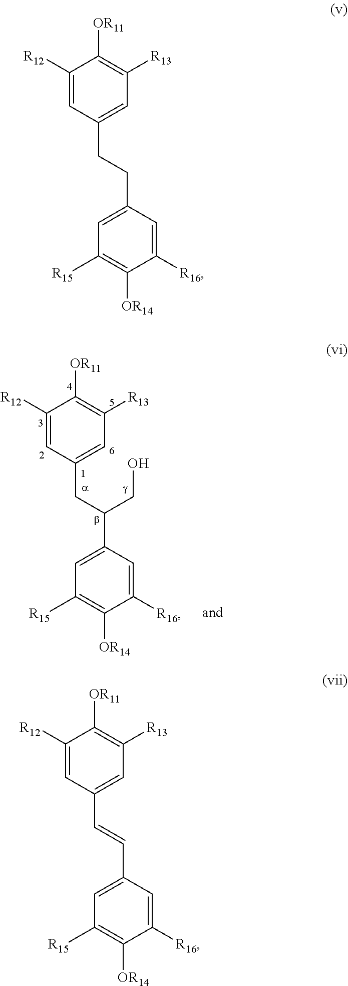

and
wherein each R₁₂, R₁₃, R₁₅ and R₁₆ is independently chosen from —H, —OH, O—CH₃, a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or aromatic oligomer, or a carbon-oxygen linkage to an aromatic monomer or aromatic oligomer,
wherein R₁₁ and R₁₄ is independently chosen from —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer or a carbon-oxygen linkage to an aromatic monomer or aromatic oligomer, and/or III) wherein at the aromatic compounds comprise at least one aromatic compound selected from the formula

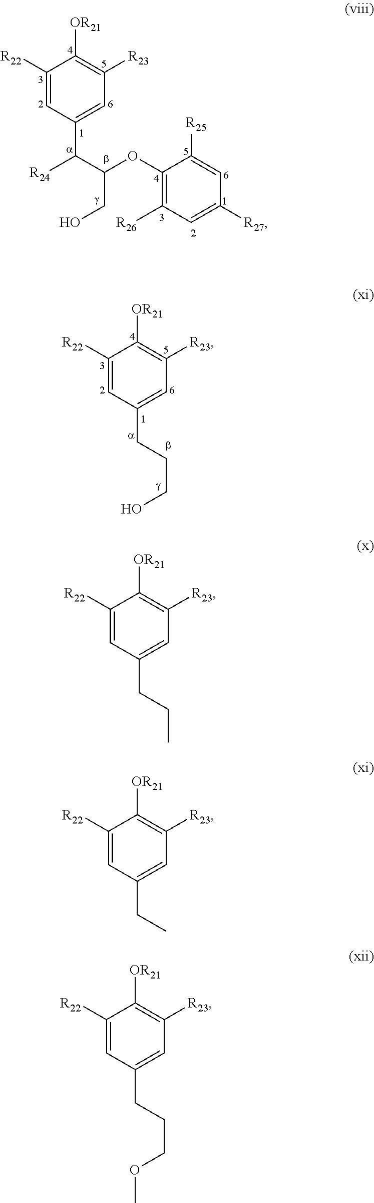

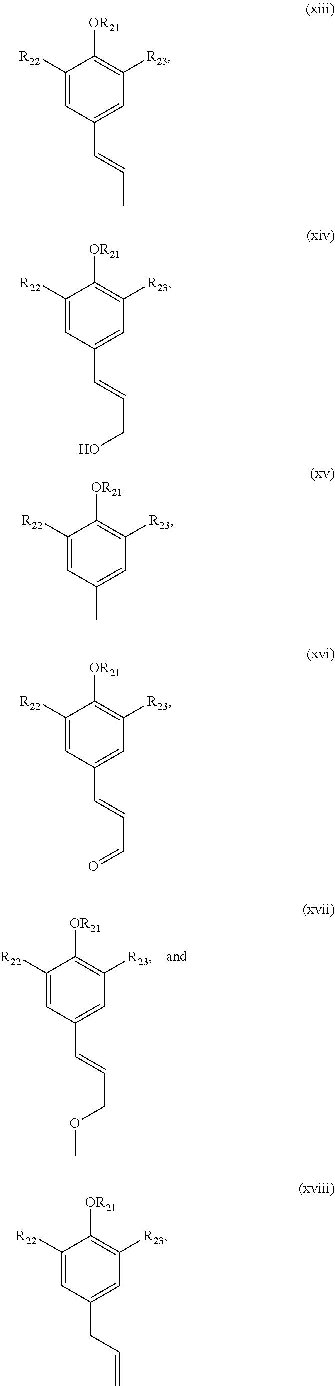

each with at least one linkage to an aromatic monomer or aromatic oligomer, and
wherein each R₂₂, R₂₃, R₂₅ and R₂₆ is independently chosen from —H, —OH, O—CH₃, a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or an aromatic oligomer, or a carbon-oxygen linkage to an aromatic monomer or aromatic oligomer,
wherein R₂₁ is independently chosen from —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer or a carbon-oxygen linkage to an aromatic monomer or aromatic oligomer,
wherein R₂₄ is independently chosen from —H, OH, or —O-Alkyl wherein the alkyl group is derived from the alcohol solvent of the process,
wherein R₂₇ is independently chosen from —H, a β-O-4 linkage to an aromatic monomer or aromatic oligomer, a β-5 linkage to an aromatic monomer or aromatic oligomer, a β-β linkage to an aromatic monomer or aromatic oligomer, a β-1 linkage to an aromatic monomer or aromatic oligomer, end-unit selected from CH₃, —CH₂CH₃, —(CH₂)₂CH₃, —CH₂CH═CH₂, —CH═CHCH₃, (CH₂)₂CH₂OH, —(CH₂)₂CHO, —CH═CHCH₂OH, —(CH₂)₂CH₂OCH₃, —CH═CHCH₂OCH₃, —(CH₂)₂CH₂OCH₂CH₃, —CH═CHCH₂OCH₂CH₃, —(CH₂)₂CH₂O(CH₂)₂CH₃, —CH═CHCH₂O(CH₂)₂CH₃, —(CH₂)₂CH₂OCH(CH₃)₂, —CH═CHCH₂OCH(CH₃)₂, —(CH₂)₂CH₂O(CH₂)₃CH₃, —CH═CHCH₂O(CH₂)₃CH₃, a carbon linkage to an aromatic monomer or an aromatic oligomer, and a fungicide also results in enhanced efficacy of the fungicide.

As used herein, “sequential use” means that first the oligosaccharide plant defense elicitor is added and subsequently the fungicide, adjuvant, surfactant or other plant defense elicitor is applied to the plant, or vice versa. “Plant pathogens” refer to organisms that cause infectious diseases in plants and include fungi, oomycetes, bacteria, viruses, viroids, virus-like organisms, phytoplasmas, protozoa, nematodes and parasitic plants. In a preferred embodiment, the plant pathogens are fungi, oomycetes, bacteria, viruses, nematodes and insects.

The majority of phytopathogenic fungi belong to the Ascomycetes and the Basidiomycetes, reproducing both sexually and asexually via the production of spores that can be spread through air (wind) or water, or can be soil borne such as zoospores that are able to live saprotrophically, carrying out the first part of their lifecycle in the soil. Deuteromycetes (Fungi imperfecti) are fungi from which only the asexual form of reproduction is known, meaning that this group of fungus produces their spores asexually. The oomycetes are not true fungi but are fungal-like organisms that use the same mechanisms as fungi to infect plants.

Non-limiting examples of phytopathogenic fungi and fungal-like organisms include Pyricularia oryzae (Magnaporthe grisea) on rice and wheat and other Pyricularia spp. on other hosts; Puccinia spp. e.g., Puccinia sorghi, Puccinia graminis fsp. tritici, Puccinia asparagi, Puccinia recondita or Puccinia arachidis, Puccinia triticina (or recondita), Puccinia striiformis and other rusts on wheat, Puccinia hordei, Puccinia striiformis and other rusts on barley, and rusts on other hosts (e.g., turf, rye, coffee, pears, apples, peanuts, sugar beet, vegetables and ornamental plants); Erysiphe cichoracearum on cucurbits (e.g., melon); Erysiphe necator (Uncinula necator) on grape, Blumeria (or Erysiphe) graminis (powdery mildew) on barley, wheat, rye and turf and other powdery mildews on various hosts, such as Sphaerotheca macularis on hops, Sphaerotheca fusca (Sphaerotheca fuliginea) on cucurbits (e.g., cucumber), Leveillula taurica on tomatoes, aubergine and green pepper, Podosphaera leucotricha on apples; Cochliobolus spp., Helminthosporium spp. (e.g., Helminthosporium turcicum, Helminthosporium carbonum, Helminthosporium mavdis or Helminthosporium sigmoideum), Drechslera spp. (Pyrenophora spp., e.g., Pyrenophora tritici-repentens or Pyrenophora teres), Rhynchosporium spp., Mycosphaerella gramninicola (Septoria tritici) and Phaeosphaeria nodorum (Stagonospora nodoruni or Septoria nodorum), Pseudocercosporella herpotrichoides and Gaeumannomyces graminis on cereals (e.g., wheat, barley, rye), turf and other hosts (e.g., Septoria lycopersici, Septoria glycines, Septoria); Cercospora arachidicola and Cercosporidium personatum on peanuts and other Cercospora spp. (e.g., Cercospora kikuchii or Cercospora zaea-maydis) on other hosts, for example, sugar beet, bananas, soya beans and rice; Botrytis spp. (e.g., Botrytis cinerea or Botryotinia fuckeliana), Botrytis cinerea (grey mold) on tomatoes, strawberries, vegetables, vines and other hosts and other Botrytis spp. on other hosts; Alternaria spp. (e.g., Alternaria brassicola or Alternaria solani) on vegetables (e.g., carrots), oil-seed rape, apples, tomatoes, potatoes, cereals (e.g., wheat) and other hosts; Venturia spp. (including Venturia inaequalis (scab) or Venturia pirina) on apples, pears, stone fruit, tree nuts and other hosts; Cladosporium spp. (e.g., Cladosporium fulvum) on a range of hosts including cereals (e.g., wheat) and tomatoes; Monilinia spp. on stone fruit, tree nuts and other hosts; Didymella spp. on tomatoes, turf, wheat, cucurbits and other hosts; Phoma spp. (e.g., Phoma betae on sugar beet and Phoma lingam on oil-seed rape), on turf, rice, potatoes, wheat and other hosts; Aspergillus spp. and Aureobasidium spp. on wheat, lumber and other hosts; Ascochyta spp. (e.g., Ascochyta pisi) on peas, wheat, barley and other hosts; Stemphylium spp. (Pleospora spp.) on apples, pears, onions and other hosts; summer diseases (e.g., bitter rot (Glomerella cingulata), black rot or frogeye leaf spot (Botryosphaeria obtusa), Brooks fruit spot (Mycosphaerellapomi), Cedar apple rust (Gymnosporangiumjuniperi-virginianae), sooty blotch (Gloeodespomigena), flyspeck (Schizothyrium pomi) and white rot (Botryosphaeria dothidea)) on apples and pears; Plasmopara viticola on vines; other downy mildews, such as Bremia lactucae on lettuce, Peronospora spp. (e.g., Peronospora manshurica or Peronospora tabacina) on soybeans, tobacco, onions and other hosts, Pseudoperonospora humuli on hops and Pseudoperonospora cubensis on cucurbits; Pythium spp. (including Pythium ultimum) on turf and other hosts (e.g., Pythium aphanidermatum); Phytophthora infestans on potatoes and tomatoes and other Phytophthora spp. on vegetables, strawberries, avocado, pepper, ornamentals, tobacco, cocoa and other hosts (e.g., Phytophthora cinnamomi, Phytophthora cactorum, Phytophthora phaseoli, Phytophthora parasitica, Phytophthora porri, Phytophthora citrophthora, Phytophthora megasperma f.sp. soiae or Phytophthora infestans); Thanatephorus cucumeris on rice and turf and other Rhizoctonia spp. on various hosts such as wheat and barley, peanuts, vegetables, cotton and turf, Sclerotinia spp. on turf, peanuts, potatoes, oil-seed rape and other hosts (e.g., Sclerotinia sclerotiorum); Sclerotium spp. on turf, peanuts and other hosts; Gibberellafujikuroi on rice; Colletotrichum spp. (e.g., Colletotrichum lindemuthianum) on a range of hosts including turf, coffee and vegetables; Laetisaria fuciformis on turf, Mycosphaerella spp. on bananas, peanuts, citrus, pecans, papaya and other hosts; Diaporthe spp. on citrus, soybean, melon, pears, lupin and other hosts; Elsinoe spp on citrus, vines, olives, pecans, roses and other hosts; Verticillium spp. (e.g., Verticillium dahliae or Verticillium albo-atrum) on a range of hosts including hops, potatoes and tomatoes; Pyrenopeziza spp. on oil-seed rape and other hosts; Oncobasidium theobromae on cocoa causing vascular streak dieback; Fusarium spp. (e.g., Fusarium nivale, Fusarium sporotrichioides, Fusarium oxysporum, Fusarium graminearum, Fusarium germinearum, Fusarium culmorum, Fusarium solani, Fusarium monilforme or Fusarium roseum), Typhula spp., Microdochium nivale, Ustilago spp., e.g., Ustilago maydis (e.g., corn smut), Urocystis spp., Tilletia spp. and Clavicepspurpurea on a variety of hosts but particularly wheat, barley, turf and maize; Ramularia spp. on sugar beet, barley and other hosts; post-harvest diseases particularly of fruit (e.g., Penicillium expansum, Penicilliumn digitatum, Penicillium italicum and Trichoderma viride on oranges, Colletotrichum musae and Gloeosporium musarum on bananas and Botrytis cinerea on grapes); other pathogens on vines, notably Eutypa lata, Guignardia bidwelhii, Phellinus igniarus, Phomopsis viticola, Pseudopeziza tracheiphila and Stereum hirsutum; other pathogens on trees (e.g., Lophodermiunm seditiosum) or lumber, notably Cephaloascusfragrans, Ceratocystis spp., Ophiostoma piceae, Penicillium spp., Trichoderma pseudokoningii, Trichoderma viride, Trichoderma harzianum, Aspergillus niger, Leptographium liindbergi and Aureobasidium pullulans; and fungal vectors of viral diseases (e.g., Polymyxa graminis on cereals as the vector of barley yellow mosaic virus (BYMV) and Polymyxa betae on sugar beet as the vector of rhizomania), Acremoniella spp., Allomyces spp., Amorphothec spp., Aspergillius spp., Blastocladiella spp., Candida spp., Chaetomium spp., Coccidioides spp., Conidiobolus spp., Coprinopsis spp., Corynascus spp., Cryphonectria spp., Cryptococcus spp., Cunninghamella spp., Curvularia spp., Debarymyces spp., Diplodia spp. (e.g., Diplodia maydis), Emericella ssp., Encephalitozoon spp., Eremothecium spp., Gaeumanomyces spp. (e.g., Gaeumanomyces graminis fsp. tritici), Geomyces spp., Gibberella spp. (e.g., Gibberella zeae), Gloeophyllum spp., Glomus spp., Hypocrea spp., Kluyveromyces spp., Lentinula spp., Leptosphaeria salvinii, Leucosporidium spp., Macrophomina spp. (e.g., Macrophomina phaseolina), Magnaportha spp. (e.g., Magnaporthe oryzae), Metharhizium spp., Mucor spp., Neurospora spp., Nectria spp. (e.g., Nectria heamatococca), Paracocidioides spp., Phaeopsheria spp., Phanerochaete spp., Phakopsora spp. (e.g., Phakopsora pachyrhizi), Phymatotrichum spp. (e.g., Phymatotrichum omnivorum), Pneumocystis spp., Pyronema spp., Rhincosporium secalis, Rhizoctonia spp. (e.g., Rhizoctonia solani, Rhizoctonia oryzae or Rhizoctonia cerealis), Rhizopus spp. (e.g., Rhizopus chinensid), Saccharomyces spp., Scerotium spp. (e.g., Scerotium rolfsii), Spizellomyces spp., Thermomyces spp., Thielaviopsis spp. (e.g., Thielaviopsis basicola), Tra metes spp., Trichophyton spp., or Yarrwia spp.

Plant diseases caused by fungi including yeasts, rusts, smuts, mildews, molds, mushrooms and toadstools that can be treated using the plant defense elicitor compositions of the disclosure are, for example: “Rust” is a fungal disease in plants, which produces reddish-brown discoloration of the stems and leaves. “Black Rot” is characterized by the darkening and decaying of leaves of fruit and vegetable plants. “Black Spot” is one of the many fungal diseases in plants. It is named “black spot” because it produces small black spots on plants. “Bottom Rot” is a fungal disease found on lettuce plants. The characteristic of this fungus is that it first affects the leaves on the lower part of the plant and then moves upward to affect the upper part. “Canker” affects the roots and bark, is found on woody trees and is notorious for causing localized damage to the bark of trees. “Cotton Ball” is notorious for attacking cranberry plants. “Crown Wart” like canker attacks on the barks of woody trees, this fungus attacks the stem of the alfalfa plants. It forms white protrusions at the base of the stem of the plant. Potato Wart” is a fungal disease that causes dark, warty, spongy excrescences in the eyes of potato tubers, similar to the crown wart in alfalfa plants. Damping Off” causes excessive moisture conditions of the seedlings. “Dry Rot” causes the drying and crumbling of timber, bulbs, potatoes or fruits. “Rhizoctinia Disease” is caused by fungi called Pellicularia and Corticium. It is often seen to affect small potatoes. “Root Rot” infects the roots causing root decay, eventually causing the plant to die. “Sclerotium Rot” is caused by Fungus of the genus Sclerotium causing the formation of sclerotia on plants. “Dutch Elm Disease” is a fungal disease affecting Elms. It spreads from one plant to another through root grafts or by the elm beetles that feed on small twigs. “Pinkroot” attacks onion plants and makes them unsuitable for consumption. “Soft Rot” is a slimy, mushy decay caused by fungi. “Yellow Spot” is characterized by a yellow spotting on the leaves of plants. “Powdery Mildews” is often specific to the host that it invades. It is normally seen on roses, lilac, English oak, zinnias, etc. “Plant Wilting” gets its name because it causes the plant it infects to wilt. The fungus invasion starts in the roots and then slowly makes its way into the stem and plugs the vascular system of the plant. “Decay” is decomposition of wood that is caused by fungi. When it attacks living plant tissue, it kills the plants.

The pine family are conifers or shrubs including the commercial important cedars, firs, hemlocks, pinons, larches, pines and spruces. In practice they are referred to as “softwood lumber” a broad industry term that refers to all commercial timber derived from gymnosperms, specifically members of the pine family (Pinaceae) and a few other coniferous families. The lignin composition among members of the Pine family (Pinaceae) is highly similar, as they are all gymnosperms (softwoods) that produce predominantly guaiacyl (G-type) lignin. While guaiacyl (G-type) lignin is the dominant lignin in gymnosperms (softwoods) like wood of the pine family, Syringyl-Guaiacyl (SG-type) lignin is typical in hardwoods like wood from birch family, beech family and willow family. The disclosure demonstrates that such lignin are useful to produce the plant defense elicitor.

Non-limiting examples of phytopathogenic bacteria include the genii Erwinia (including Erwinia amylovora, causing fire blight on pears), Pseudomonas (including Pseudomonas syringae), Xanthomonas (including Xanthomonas orizae, Xanthomonas citri, Xanthomonas fuscans (citrus cancer) and Xanthomonas fragariae) and Ralstonia.

Non-limiting examples of phytopathogenic viruses include Cucumber Mosaic Virus, Barley Yellow Mosaic Virus, Strawberry Mild Yellow Edge Virus, Strawberry Latent Ringspot Virus, Beet Necrotic Yellow Vein Virus and Potato Virus Y.

Phytopathogenic insects that can be targeted by application of the compositions of the disclosure include aphids, beetles, bugs, hoppers, locusts, mites, ants, ticks, trips, whiteflies, rootworms, maggots, weevils, (stem)borers, caterpillars, butterflies, leaf-rollers, leaf-miners, etc.

“Plant protection” as used herein refers to the activation of mechanisms aimed at controlling or reducing the pathogens and/or to minimize their effects on the plant. Plant protection can be achieved by killing the pathogens, by delaying their growth and/or reproduction, by reducing sporulation, etc. According to another aspect of the disclosure, there is provided a method for protecting plants against (infection by) plant pathogens, characterized in that an effective and substantially non-phytotoxic amount of a composition hereof is applied to the plants. The expression “effective and non-phytotoxic amount” means an amount of plant defense elicitor composition hereof that is sufficient to induce control or destruction of the plant pathogens present or liable to appear on the plants, and that does not entail any appreciable symptom of phytotoxicity for the plants. Such an amount can vary within a wide range depending on the plant pathogen to be controlled, the type of plant, the climatic conditions and the compounds included in the composition hereof. This amount can be determined by systematic field trials that are within the capabilities of a person skilled in the art.

In a particularly preferred embodiment, the fungicide in the composition of the disclosure is applied at a reduced rate. Preferably, the rate of the fungicide is reduced by at least a factor 2, 3, 4, 5, 6, 7, 8, 9, 10, 15, 25, 40, 50, 60, 70, 80, 90, or 100 when compared to the recommended rate, or is reduced by 10, 20, 30, 40, 50, 60, 70, 80, 90, or even 95% or more of the recommended dosage for the plant and/or conditions. More preferably, the rate of the fungicide is reduced by 50% to 90%, 60% to 90%, 70% to 90%, 80% to 90%, 60% to 80%, or 60% to 70% of the recommended rate for the plant and/or conditions.

Application of the composition hereof can be carried out in accordance with techniques well known to persons skilled in the art. The composition hereof can be applied to the whole plant, or to leaves, flowers, fruits, seeds and/or roots of the plant, as well as to the soil or inert substrate wherein the plant is growing or in which it is desired to grow (e.g., inorganic substrates like sand, rockwool, glasswool; expanded minerals like perlite, vermiculite, zeolite or expanded clay), pumice, pyroclastic materials or stuff, synthetic organic substrates (e.g., polyurethane), organic substrates (e.g., peat, composts, tree waste products like coir, wood fiber or chips, tree bark) or to a liquid substrate (e.g., floating hydroponic systems, Nutrient Film Technique, Aeroponics). The application can be done by spraying, drenching, soaking, dipping, injection, etc., or via fertigation systems.

It can also be useful to apply the compositions of the disclosure to propagation material such as tubers or rhizomes, but also seeds, seedlings or seedlings pricking out and plants or plants pricking out. The compositions of the disclosure can also be applied post-harvest to control decay. Among the plants that can be protected by the method according to the disclosure, mention can be made of cotton; flax; vine; fruit or vegetable crops such as rosaceae sp. (for instance, pip fruit such as apples and pears, but also stone fruit such as apricots, almonds and peaches), ribesioidae sp., juglandaceae sp., betulaceae sp., anacardiaceae sp., fagaceae sp., moraceae sp., oleaceae sp., actinidaceae sp., lauraceae sp., musaceae sp. (for instance, banana trees and plantins), rubiaceae sp., theaceae sp., sterculiceae sp., vitaceae sp., rutaceae sp. (for instance, lemons, oranges and grapefruit); solanaceae sp. (for instance, tomatoes), liliaceae sp., asteraceae sp. (for instance, lettuces), umbelliferae sp., cruciferae sp., chenopodiaceae sp., cucurbitaceae sp., papilionaceae sp. (for instance, peas), rosaceae sp. (for instance, strawberries); major crops such as graminae sp. (for instance, maize, lawn or cereals such as wheat, rice, barley and triticale), asteraceae sp. (for instance, sunflower), brassicaceae sp. (for instance, rapeseed and colza), fabacae sp. (for instance, peanuts), papilionaceae sp. (for instance, soybean), solanaceae sp. (for instance, tomatoes and potatoes), chenopodiaceae sp. (for instance, beetroots); horticultural and forest crops; as well as genetically modified homologues of these crops.

As used herein, the term “monophenolic compounds” means molecules with one phenolic group. If the molecules result from the chemical modification of lignin, they are referred to as “lignin-derived monophenolics,” “lignin-derived monomers,” “lignin monomers,” or “phenolic monomers.” These terms are used interchangeably. Chemical modification herein means depolymerization and/or partial reduction. The lignin-derived monophenolics comprise compounds having the formulae:

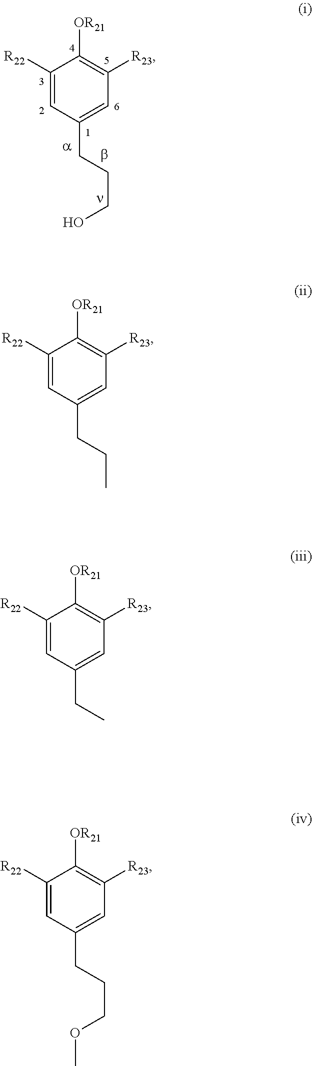

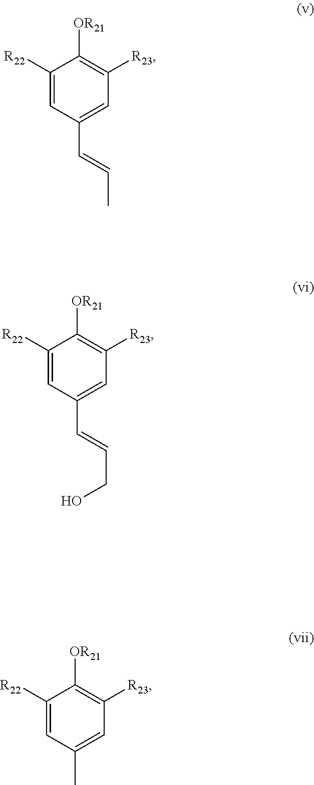

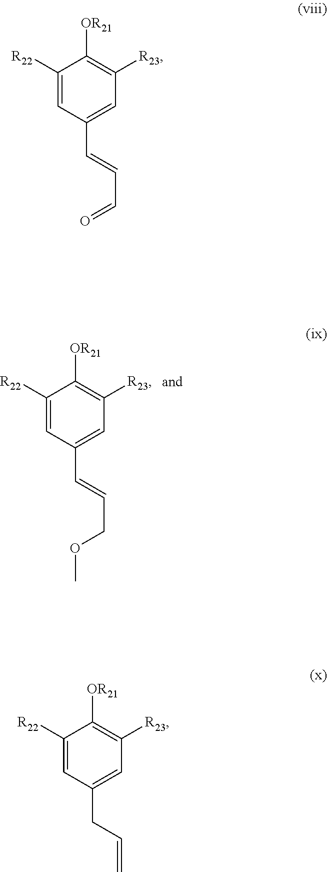

each with at least one linkage to an aromatic monomer or aromatic oligomer and
wherein each R₂₂, R₂₃, R₂₅ and R₂₆ is independently chosen from —H, —OH or O—CH₃,
wherein R₂₁ is —H
wherein R₂₄ is independently chosen from —H or OH,
wherein R₂₇ is independently chosen from —H or an end-unit selected from CH₃, —CH₂CH₃, —(CH₂)₂CH₃, —CH₂CH═CH₂, —CH═CHCH₃, (CH₂)₂CH₂OH, —(CH₂)₂CHO, —CH═CHCH₂OH, —(CH₂)₂CH₂OCH₃, —CH═CHCH₂OCH₃, —(CH₂)₂CH₂OCH₂CH₃, —CH═CHCH₂OCH₂CH₃, —(CH₂)₂CH₂O(CH₂)₂CH₃, —CH═CHCH₂O(CH₂)₂CH₃, —(CH₂)₂CH₂OCH(CH₃)₂, —CH═CHCH₂OCH(CH₃)₂, —(CH₂)₂CH₂O(CH₂)₃CH₃, —CH═CHCH₂O(CH₂)₃CH₃

Specific examples of the phenolic monomers are:

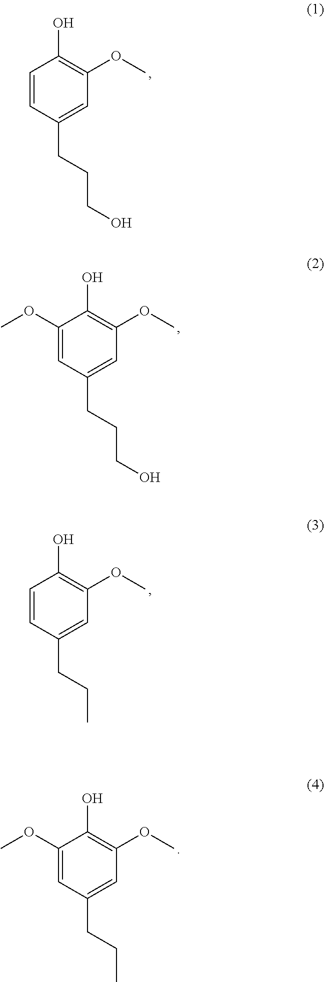

(1) is referred to as “4-(3-hydroxy-propyl)-guaiacol,” “propanol-guaiacol,” or simply “propanol-G”;
(2) is referred to as “4-(3-hydroxy-propyl)-syringol,” “propanol-syringol,” or simply “propanol-S”;
(3) is referred to as “4-n-propyl-guaiacol,” “propyl-guaiacol,” or simply “propyl-G”; and
(4) is referred to as “4-n-propyl-syringol,” “propyl-syringol,” or simply “propyl-S.”

As used herein, the term “diphenolic compounds” means molecules with two phenolic centers chemically linked to each other. Thus, having a DP of 2 and two phenol molecules. If the molecules result from the chemical modification of lignin, they are referred to as “lignin-derived diphenolics,” “lignin-derived dimers,” or “phenolic dimers.” These terms are used interchangeably. Chemical modification herein means depolymerization and/or partial reduction.

As used herein, the term “triphenolic compounds” or trimers means molecules with three phenolic centers chemically linked to each other. Thus, having a DP of 3 and three phenol molecules. If the molecules result from the chemical modification of lignin, they are referred to as “lignin-derived triphenolics,” “lignin-derived trimers,” or “phenolic trimers.”

As used herein, molecules with three phenolic centers chemically linked to each other resulting from the chemical modification of lignin are referred to as “lignin-derived triphenolics,” “lignin-derived trimers,” or “phenolic trimers.” These terms are used interchangeably.

As used herein, the term “tetraphenolic compounds” or tetramers means molecules with four phenolic centers chemically linked to each other. Thus, having a DP of 4 and four phenol molecules. If the molecules result from the chemical modification of lignin, they are referred to as “lignin-derived tetraphenolics,” “lignin-derived tetramers,” or “phenolic tetramers.”

As used herein, molecules with four phenolic centers chemically linked to each other resulting from the chemical modification of lignin are referred to as “lignin-derived tetraphenolics,” “lignin-derived tetramers,” or “phenolic tetramers.” These terms are used interchangeably.

As used herein, the term “polyphenolic compounds” means molecules resulting from the chemical modification of lignin. Hence, they are referred to as “lignin-derived polyphenolics,” “lignin-derived oligomers,” or “phenolic oligomers.” These terms are used interchangeably. Chemical modification herein means depolymerization and/or partial reduction.

As used herein, the term “phenolic compounds” and “phenolic products mixture” are used interchangeably to indicate the mixture comprising monophenolic compounds, diphenolic compounds, triphenolic compounds, tetraphenolic compounds and other phenolic oligomers.

As used herein, the term “hemicellulose-derived polyols” or simply “polyols” means aliphatic alcohols comprising at least two hydroxyl groups. As used herein, the term “hemicellulose-derived polyols” or “polyols” does not include monosaccharides or oligosaccharides. Hemicellulose-derived polyols include sugar alcohols derived from hydrogenation of monosaccharides. The polyols result from the chemical modification of hemicellulose. Chemical modification herein means hydrolysis and hydrogenation. Hemicellulose-derived polyols primarily include xylitol, Arabitol, dulcitol, mannitol, sorbitol, ethylene glycol, glycerol. The term “C5 polyols” is used to indicate the group of polyols comprising 5 carbon atoms, such as xylitol and Arabitol. The term “C6 polyols” is used to indicate the group of polyols comprising 6 carbon atoms, such as dulcitol, mannitol, and sorbitol. Likewise, the term “C5 sugars” is used to indicate the group of sugars comprising 5 carbon atoms, such as xylose and arabinose. The term “C6 sugars” is used to indicate the group of sugars comprising 6 carbon atoms, such as glucose, mannose, and galactose.

The term “hemicellulose-derived oligosaccharides,” “hemicellulose oligomers” or simply “oligosaccharides” is used to indicate molecules comprising two or more saccharide monomers or saccharide-derived monomers, linked to each other by a glycosidic bond.

The term “oligosaccharides” is used to denote saccharide oligomers with a non-reduced terminal saccharide group as well as molecules with a reduced terminal saccharide group.

Examples of such oligosaccharides include, but are not limited to.

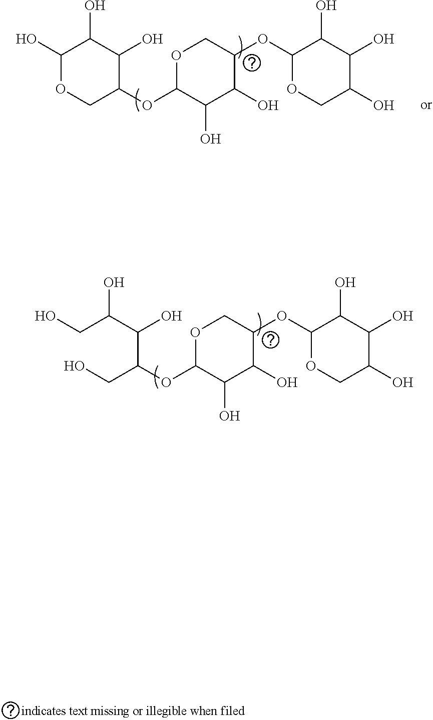

with n between 0-7, for example, 0-3.

The term “unstable compounds” is used to refer to compounds that are unstable under the reaction conditions of the lignin depolymerization process or in the formulation of the ISR product, and that cause unwanted side-reactions, such as recondensation. Unstable compounds typically bear a C═O or C═C functional group. Examples of such unstable compounds derived from carbohydrates include, but are not limited to, xylose, glucose, furfural, and hydroxymethylfurfural. Examples of unstable compounds derived from lignin include, but are not limited to, coniferyl alcohol, sinapyl alcohol, phenolic compounds with C2-aldehyde substituents, and so-called Hibbert's ketones. These unstable compounds can be transformed to “stable compounds” by transforming the C═O and/or C═C functional groups, e.g., through hydrogenation. The term “stable compounds” is used to refer to compounds that are stable under the reaction conditions or in the formulation of the ISR product, and that do not cause unwanted side-reactions, such as recondensation. Examples of stable compounds derived from carbohydrates include, but are not limited to, xylitol, Arabitol, dulcitol, mannitol, sorbitol, ethylene glycol, glycerol. Examples of stable compounds derived from lignin include, but are not limited to, 4-n-propanolsyringol, 4-n-propanolguaiacol, 4-n-propylsyringol, 4-n-propylguaiacol.

Non-limiting examples of “lignin” sources are from woody plants (vascular plants (tracheophytes) which includes most trees, shrubs) of the group of softwoods (softwood lumber) and hardwoods (oak, maple, birch), non-woody plants such as grasses (wheat straw, rice straw, switchgrass), herbaceous plants (bamboo, sugar cane), seed coats (nuts, legume pulses, beans), flax stems and hemp stem, jute and bagasse (sugarcane residue).

Crops subjected to the plant defense elicitor of the disclosure are not particularly limited and any general cultivated plants can be subjected. Examples thereof include the Poaceae plants (such as rice, barley, wheat, corn, oat or lawn grass), the Solanaceae plants (such as tomato, eggplant or potato), the Cucurbitaceae plants (such as cucumber, melon or pumpkin), the Leguminosae plants (such as pea, soybean, kidney bean, alfalfa, peanut, fava bean), the Brassicaceae plants (such as daikon radish, Chinese cabbage, cabbage, komatsuna, rape blossoms, bok choy or A. thaliana), the Rosaceae plants (such as strawberry, apple or pear), the Moraceae (such as mulberry), the Malvaceae (such as cotton), the Umbelliferae (such as carrot, parsley or celery), the Liliaceae (such as green onion, onion or asparagus), the Compositae (such as burdock, sunflower, chrysanthemum, crown daisy, safflower, lettuce) and the Vitaceae (such as grape).

Since the reaction that gives rise to plant disease resistance is generally nonspecific to pathogens, all the plant diseases caused by fungus, bacteria and viruses are included as subject diseases. Examples thereof include diseases caused by Magnaporthe grisea, Cochliobolus miyabeanus, Pseudomonas syringae pv. maculicola, Spongospora subterranea, Phytophthora infestans, Peronospora manshurica, Eryshiphe graminis f. sp. hordei, Eryshiphe graminis f. sp. tritici, Gibberella zeae, Mycosphaerella pinodes, Sclerotinia borealis, Puccinia recondita, Ustilago maydis, Ceratobasidium gramineum, Rhizoctonia solani, Rhizoctonia solani, Alternaria solani, Cercospora kikuchii, Fusarium oxysporum f. sp. batatas, Fusarium oxysporum f. sp. melonis, Fusarium oxysporum f. sp. lactucae, Fusarium oxysporum f. sp. lycopersici, Fusarium oxysporum f. sp. spinaciae, Verticillium dahliae, Plasmodiophora brassicae, Pythium debaryanum, Botrytis cinerea, Colletotrichum phomoides, Hordeum vulgare, Pseudomas syringae pv. syringae, Erwiniasubsp. atroseptica, Xanthomonas campestris pv. oryzae, Streptomyces scabies, Soil-borne wheat mosaic virus, Soybean mosaic virus, Alfalfa mosaic virus and Potato leafroll virus.

The plant defense elicitor hereof can be used on plants in any form, such as solution, powder, granule, emulsion, wettable powder, oil, aerosol, flowable by mixing the lignin derived dimers with appropriate additives such as zinc and/or copper, bicarbonates, carbocation scavenger or polyhydric alcohols. Optionally, the pH thereof can be adjusted by adding buffer, and properties such as penetration properties to plants or spreading properties can be modified by adding a spreading agent, surfactant such as plant lecithins, such as lyso-lecithin, or the like and amino acids such as branched-chain amino acids, proline, glutamic acid, aspartic acid, and histidine.

In view of the foregoing discussion, the present application also provides aspects and embodiments as set forth in the following statements (1′ to 27′):

Statement 1′ A phytopharmaceutical or agrochemical composition, having an effective dose of plant defense elicitor compound(s) that are lignin-derived phenolic oligomers with a DP of 2 to 8 (preferably 2-4), or synthesized structural similar compounds.

Statement 2′ The composition of statement 1′, wherein the composition has a pH in the range of 4 to 10, preferably in the range of 5 to 8.

Statement 3′ The composition of any one of the statements 1′ to 2′, wherein the lignin-derived phenolic oligomers have a pH in the range of 4.0 to 6.0 in origin.

Statement 4′ The composition of any one of the statements 1′ to 3′, wherein defense elicitor compounds are 0.5 to 30 wt % by dry weight of aromatic compounds, preferably 1 to 20 wt % by dry weight of aromatic compounds, more preferably 2 to 10 wt % of the composition in dry state.

Statement 5′ The composition of any one of the statements 1′ to 4′, wherein defense elicitor compounds are of the group of lignin-derived dimerics (diphenolic) compounds, lignin-derived trimeric (triphenolic) compounds, lignin-derived tetrameric (tetraphenolic) compound or a combination thereof (FIGS. 2A-2D).

Statement 6′ The composition of any one of the statements 1′ to 5′, wherein lignin-derived phenolic oligomers are from depolymerized lignin or decomposed lignin.

Statement 7′ The composition of any one of the statements 1′ to 6′, wherein lignin-derived phenolic oligomers are from a lignin that is depolymerized or decomposed by non-catalytic thermosolvolytic depolymerization (FIG. 1B), reductive catalytic fractionation (RCF) (FIG. 1A) of lignocellulose.

Statement 8′ The composition of any one of the statements 1′ to 7′, wherein the lignin is from a lignocellulose biomass, e.g., flax shives, wood chips, pine, spruce or poplar sawdust.

Statement 9′ The composition of any one of the statements 1′ to 8′, further comprising an ingredient selected from the group consisting of a surfactant, a biosurfactant, a penetration enhancer, a dispersing agent, an emulsifier and a carrier or a combination thereof.

Statement 10′ The composition of any one of the statements 1′ to 9′, further comprising a second plant defense elicitor, e.g., of the group consisting of alginate, hexokinase, laminarin, sodium silicate, silicon, oligo-galacturonan, cellodextrin and/or chito-oligosaccharide or a plant defense elicitor selected from the group consisting of pectin fragment, oligogalacturonide, cellobiose, xyloglucan, non-branched P-1,3-glucan, chitin fragment, arabinose, arabinan, rhamnose, homogalacturonan, rhamnogalacturonan I and II, xylogalacturonan, starch, and combinations thereof.

Statement 11′ The composition of any one of the statements 1′ to 10′, further comprising a fungicide, antimicrobial, an insecticidal, or an antiviral.

Statement 12′ The composition of any one of the statements 1′ to 11′, further comprising a fungicide selected from the group consisting of phosphonates, benzamides, carbamates, dithiocarbamates, phthalimides, triazoles, quinolines, sulfur, and cyanoimidazoles or a fungicide selected from the group consisting of 2,6-dichloro-N-[3-chloro-5-(trifluoromethyl)-2-pyridinylmethyl]benzamide; propyl 3-(dimethylamino)propylcarbamate hydrochloride; (2RS,3SR)-1-[3-(2-chlorophenyl)-2,3-epoxy-2-(4-fluorophenyl)propyl]-1H-1,2,4-triazole; 5,7-dichloro-4-quinolyl 4-fluorophenyl ether; sulfur; 4-chloro-2-cyano-N,N-dimethyl-5-(4-methylphenyl)-1H-imidazole-1-sulfonamide; N-(trichloromethylthio)phthalimide; manganese ethylenebis(dithiocarbamate) (polymeric) complex with zinc salt and methyl (E)-methoxyimino-{(E)-α-[1-(α,α,α-trifluoro-m-tolyl)ethylideneaminooxy]-o-tolyl}acetate; or a combination thereof.

Statement 13′ The composition of any one of the statements 1′ to 12′, further comprising a polymerization inhibitor of the group of a carbocation scavenger, aromatic scavengers and a polyhydric alcohol or a combination thereof.

Statement 14′ The composition of any one of the statements 1 to 12, further comprising a polymerization inhibitor wherein the polymerization inhibitor is a compound of the group consisting of citric acid, salicylic acid, 2-naphthol, phenolic acids (e.g., vanillic acid, syringic acid), ethylene glycol, glycerol, mannitol (C₆H₁₄O₆), sorbitol (C₆H₁₄O₆), xylitol (C₅H₁₂O₅), erythritol, and maltitol (C₁₂H₂₄O₁₁).

Statement 15′ The composition of any one of the statements 1′ to 14′, further comprising a stabilizing agents of the group consisting of 1,4-butanediol, 2-hydroxy-1-naphthoic acid, 2-naphthol, 2-naphthol-7-sulfonat, 2-naththol, 3-hydroxy-2-naphthoic acid, 4-hydroxybenzoic acid, 6-hydroxy-2-naphthoic acid, ascorbic acid, bovine serum albumin, citric acid, salicylic acid, cysteine, dimethylphloroglucinol, histidine, mannitol, o-dihydroxybenzene, p-benzenediol, phloroglucinol, resorcinol, soy protein isolate, syringic acid and vanillic acid so to prevent repolymerization.

Statement 16′ The composition of any one of the statements 1 to 15, wherein the phenolic oligomers comprise one or more aromatic compounds, with a DP of 2 to 8 (preferably 2-4) or one or more aromatic compounds, with a molecular mass of between 180 g/mol to 1000 g/mol (preferably between 230 g/mol to 650 g/mol), and wherein the aromatic compounds comprise and/or I) at least one aromatic compound selected from the formulae

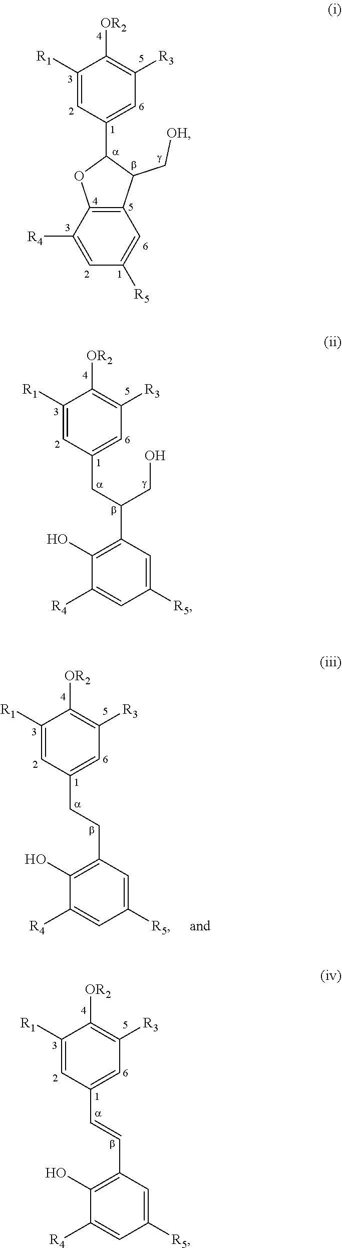

and
wherein each R₁, R₃, and R₄ is independently chosen from —H, —OH, O—CH₃, a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or aromatic oligomer, or a carbon-oxygen linkage to an aromatic monomer or aromatic oligomer,
wherein R₂ is —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer, or a carbon-oxygen linkage to an aromatic monomer or aromatic oligomer,
wherein R₅ is selected from —H, a β-O-4 linkage to an aromatic monomer or aromatic oligomer, a β-5 linkage to an aromatic monomer or aromatic oligomer, a β-β linkage to an aromatic monomer or aromatic oligomer, a β-1 linkage to an aromatic monomer or aromatic oligomer, an end-unit selected from CH₃, —CH₂CH₃, —(CH₂)₂CH₃, —CH₂CH═CH₂, —CH═CHCH₃, —(CH₂)₂CH₂OH, —(CH₂)₂CHO, —CH═CHCH₂OH, —(CH₂)₂CH₂OCH₃, —CH═CHCH₂OCH₃, —(CH₂)₂CH₂OCH₂CH₃, —CH═CHCH₂OCH₂CH₃, —(CH₂)₂CH₂O(CH₂)₂CH₃, —CH═CHCH₂O(CH₂)₂CH₃, —(CH₂)₂CH₂OCH(CH₃)₂, —CH═CHCH₂OCH(CH₃)₂, —(CH₂)₂CH₂O(CH₂)₃CH₃, —CH═CHCH₂O(CH₂)₃CH₃, a carbon linkage to an aromatic monomer or aromatic oligomer; and/or
II) at least one aromatic compound selected from the formulae

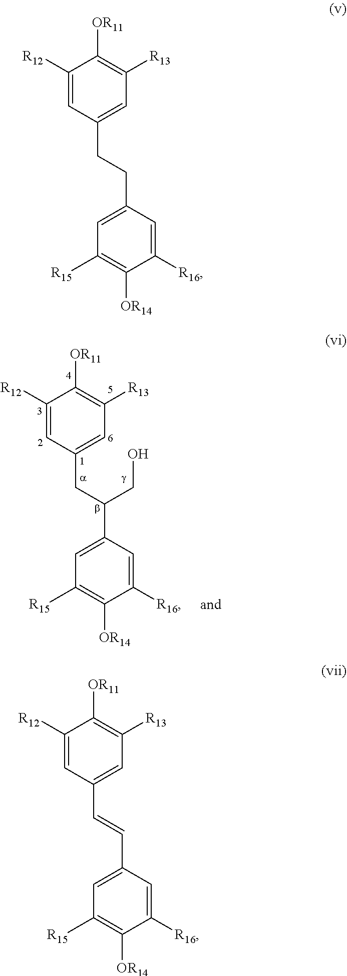

and
wherein each R₁₂, R₁₃, R₁₅ and R₁₆ is independently chosen from —H, —OH, O—CH₃, a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or aromatic oligomer, or a carbon-oxygen linkage to an aromatic monomer or aromatic oligomer,
wherein R₁₁ and R₁₄ is independently chosen from —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer or a carbon-oxygen linkage to an aromatic monomer or aromatic oligomer, and/or III) wherein at the aromatic compounds comprise at least one aromatic compound selected from the formula

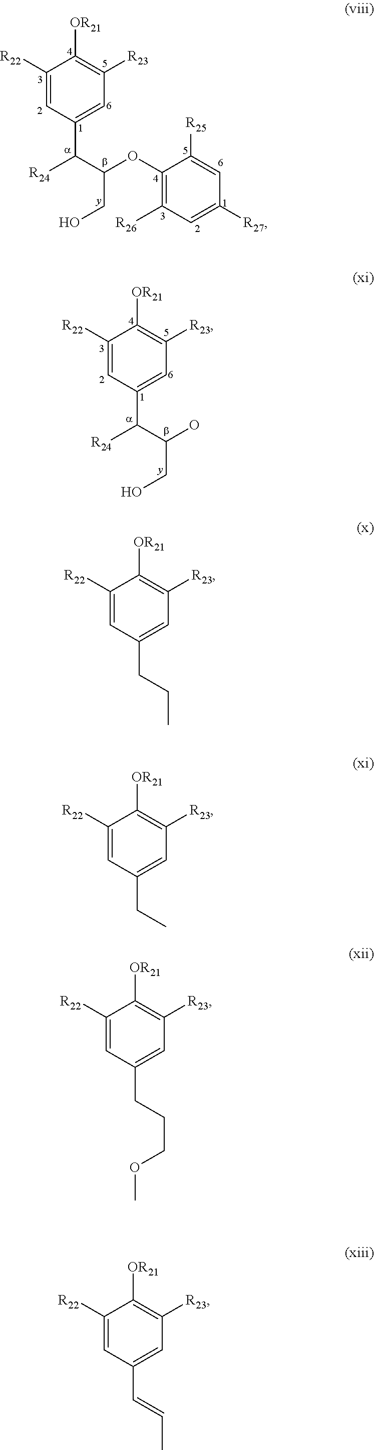

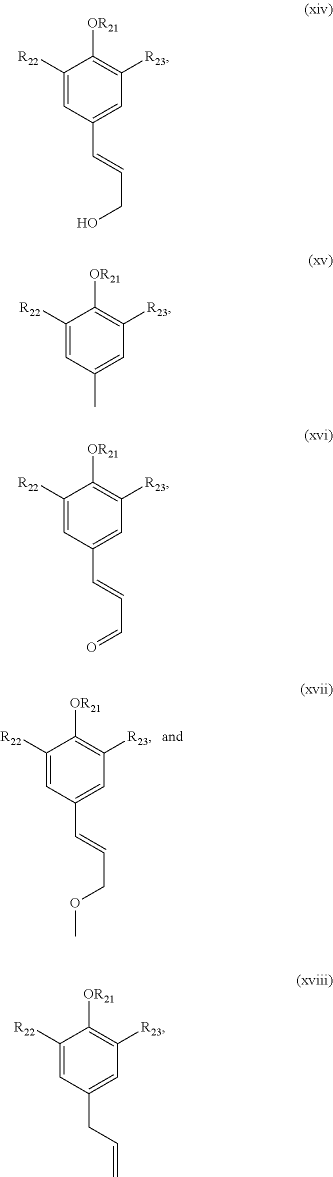

each with at least one linkage to an aromatic monomer or aromatic oligomer and
wherein each R₂₂, R₂₃, R₂₅ and R₂₆ is independently chosen from —H, —OH, O—CH3, a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or an aromatic oligomer, or a 4 carbon-oxygen linkage to an aromatic monomer or aromatic oligomer,
wherein R₂₁ is independently chosen from —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer or a carbon-oxygen linkage to an aromatic monomer or aromatic oligomer,
wherein R₂₄ is independently chosen from —H, OH, or —O-Alkyl wherein the alkyl group is derived from the alcohol solvent of the process, wherein R₂₇ is independently chosen from —H, a β-O-4 linkage to an aromatic monomer or aromatic oligomer, a β-5 linkage to an aromatic monomer or aromatic oligomer, a β-β linkage to an aromatic monomer or aromatic oligomer, a β-1 linkage to an aromatic monomer or aromatic oligomer, end-unit selected from CH₃, —CH₂CH₃, —(CH₂)₂CH₃, —CH₂CH═CH₂, —CH═CHCH₃, —(CH₂)₂CH₂OH, —(CH₂)₂CHO, —CH═CHCH₂OH, —(CH₂)₂CH₂OCH₃, —CH═CHCH₂OCH₃, —(CH₂)₂CH₂OCH₂CH₃, —CH═CHCH₂OCH₂CH₃, —(CH₂)₂CH₂O(CH₂)₂CH₃, —CH═CHCH₂O(CH₂)₂CH₃, —(CH₂)₂CH₂OCH(CH₃)₂, —CH═CHCH₂OCH(CH₃)₂, —(CH₂)₂CH₂O(CH₂)₃CH₃, —CH═CHCH₂O(CH₂)₃CH₃, a carbon linkage to an aromatic monomer or an aromatic oligomer.

Statement 17′ The composition of any one of the statements 1 to 16, wherein the phenolic oligomers are diphenolics comprising compounds wherein the aromatic compounds have 2 aromatic groups or a DP of 2.

Statement 18′ The composition of any one of the statements 1′ to 17′, for use in a method of promoting induced systemic resistance in a plant or for inducing latent host defenses in a plant by priming of the intrinsic disease resistance mechanisms of a plant.

Statement 19′ Use of the composition of any one of the statements 1′ to 18′, on a plant or parts thereof wherein the latent host defenses are activated preventive to or in the event of an attack by a phytopathogenic pathogen or pest.

Statement 20′ Use of the composition of any one of the statements 1′ to 18′, on a plant or parts thereof wherein the latent host defenses are activated preventive to or in the event of an attack by a phytopathogenic pathogen or pest wherein the phytopathogenic pathogen or pest is selected from the group of a fungi, a bacterium, and insect.

Statement 21′ Use of the composition of any one of the statements 1′ to 18′, on a plant or parts thereof wherein the latent host defenses are activated preventive to or in the event of an attack by a phytopathogenic of the group consisting of fungi, bacteria, viruses, viroids, mycoplasma-like organisms, protozoa, insects, acari, and nematodes.

Statement 22′ Use of the composition of any one of the statements 1′ to 18′, on a plant or parts thereof wherein the latent host defenses are activated preventive to or in the event of abiotic stress.

Statement 23′ Use of the composition of any one of the statements 1′ to 18′, on a plant or parts thereof to prime the intrinsic resistance mechanisms of a plant for stronger or faster induced plant defense preventive to or in the event of an attack by a phytopathogenic pathogen or pest or of abiotic stress, as compared to control or other plant defense inducers.

Statement 24′ Use of the composition of any one of the statements 1′ to 15′, wherein the composition is a foliar spray agent.

Statement 25′ Use of the composition of any one of the statements 1′ to 21′, wherein the composition is a root drench.

Statement 26′ A method for promoting induced systemic resistance, for inducing latent host defenses, or for priming the intrinsic resistance mechanisms in a plant, comprising applying to a plant or a plant part, the composition of any one of the statements 1′ to 18′.

Statement 27′ A method for promoting induced systemic resistance, for inducing latent host defenses or for priming the intrinsic resistance mechanisms in a plant, comprising by spraying on the plant or contacting the roots of the plant with the composition of any one of the statements 1′ to 21′.

In view of the foregoing discussion, the present application also provides aspects and embodiments as set forth in the following statements (1* to 17*):

Statement 1* A composition for protecting plants against plant pests comprising a plant defense elicitor comprising, consisting of or essentially consisting of as active ingredient one or more aromatic compounds, with a DP of 2 to 8 (preferably 2-4) or one or more aromatic compounds, with a molecular mass between 180 g/mol to 1000 g/mol (preferably between 230 g/mol to 650 g/mol) (FIGS. 2A-2D), and wherein the aromatic compounds comprise and/or I) at least one aromatic compound selected from the formulae

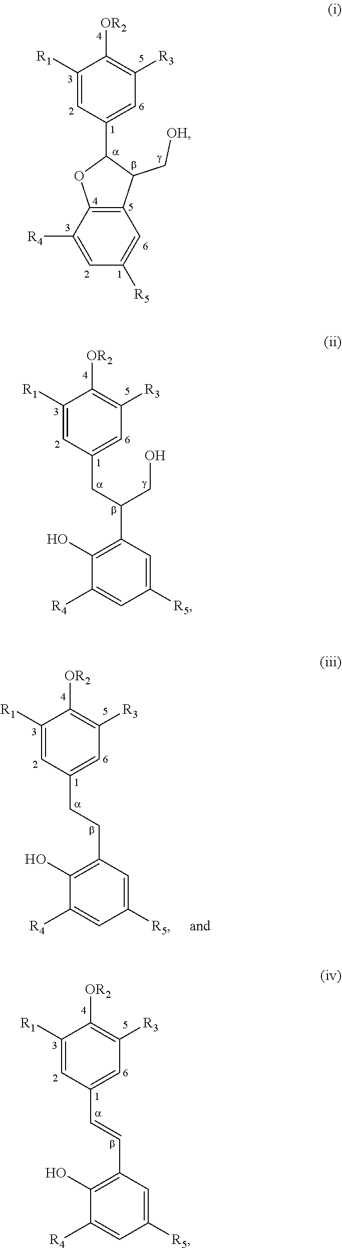

and
wherein each R₁, R₃, and R₄ is independently chosen from —H, —OH, O—CH3, a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or aromatic oligomer, or a carbon-oxygen linkage to an aromatic monomer or aromatic oligomer,
wherein R₂ is —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer, or a carbon-oxygen linkage to an aromatic monomer or aromatic oligomer,
wherein R₅ is selected from —H, a β-O-4 linkage to an aromatic monomer or aromatic oligomer, a β-5 linkage to an aromatic monomer or aromatic oligomer, a β-β linkage to an aromatic monomer or aromatic oligomer, a β-1 linkage to an aromatic monomer or aromatic oligomer, an end-unit selected from CH₃, —CH₂CH₃, —(CH₂)₂CH₃, —CH₂CH═CH₂, —CH═CHCH₃, —(CH₂)₂CH₂OH, —(CH₂)₂CHO, —CH═CHCH₂OH, —(CH₂)₂CH₂OCH₃, —CH═CHCH₂OCH₃, —(CH₂)₂CH₂OCH₂CH₃, —CH═CHCH₂OCH₂CH₃, —(CH₂)₂CH₂O(CH₂)₂CH₃, —CH═CHCH₂O(CH₂)₂CH₃, —(CH₂)₂CH₂OCH(CH₃)₂, —CH═CHCH₂OCH(CH₃)₂, —(CH₂)₂CH₂O(CH₂)₃CH₃, —CH═CHCH₂O(CH₂)₃CH₃,
and/or II) the aromatic compounds comprise at least one aromatic compound selected from the formulae

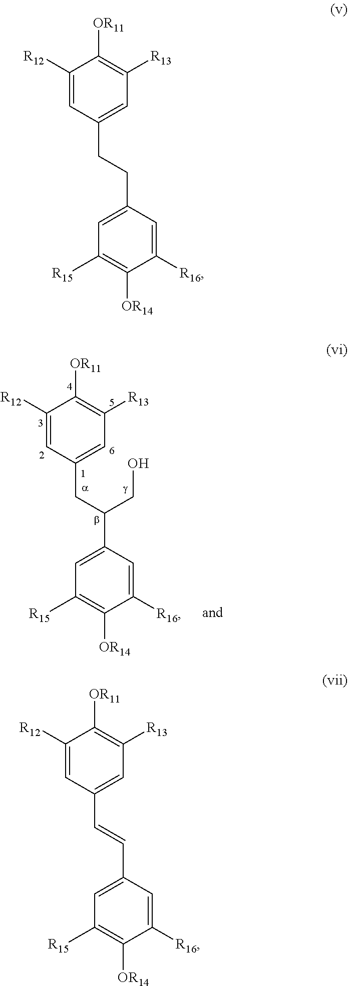

and
wherein each R₁₂, R₁₃, R₁₅ and R₁₆ is independently chosen from —H, —OH, O—CH3, a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or aromatic oligomer, or a carbon-oxygen linkage to an aromatic monomer or aromatic oligomer,
wherein R₁₁, and R₁₄ is independently chosen from —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer or a carbon-oxygen linkage to an aromatic monomer or aromatic oligomer, and/or
wherein at the aromatic compounds comprise at least one aromatic compound selected from the formula

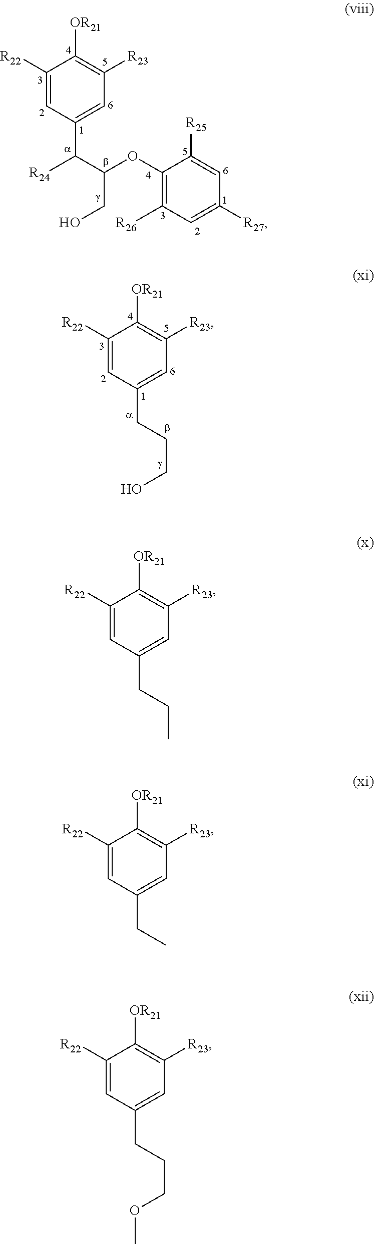

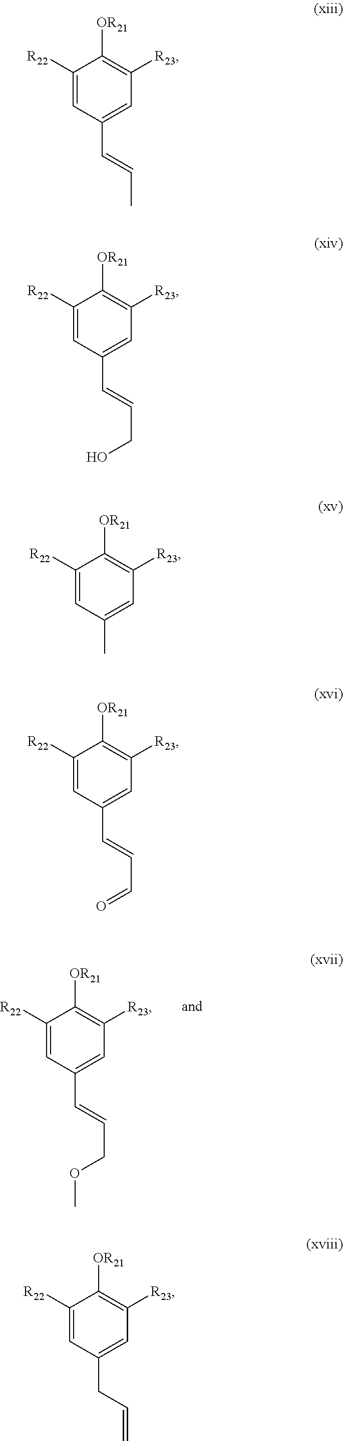

each with at least one linkage to an aromatic monomer or aromatic oligomer, and
wherein each R₂₂, R₂₃, R₂₅ and R₂₆ is independently chosen from —H, —OH, O—CH₃, a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or an aromatic oligomer, or a 4 carbon-oxygen linkage to an aromatic monomer or aromatic oligomer,
wherein R₂₁ is independently chosen from —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer or a carbon-oxygen linkage to an aromatic monomer or aromatic oligomer,
wherein R₂₄ is independently chosen from —H, OH, or —O-Alkyl wherein the alkyl group is derived from the alcohol solvent of the process,
wherein R₂₇ is independently chosen from —H, a β-O-4 linkage to an aromatic monomer or aromatic oligomer, a β-5 linkage to an aromatic monomer or aromatic oligomer, a β-β linkage to an aromatic monomer or aromatic oligomer, a β-1 linkage to an aromatic monomer or aromatic oligomer, end-unit selected from CH₃, —CH₂CH₃, —(CH₂)₂CH₃, —CH₂CH═CH₂, —CH═CHCH₃, —(CH₂)₂CH₂OH, —(CH₂)₂CHO, —CH═CHCH₂OH, —(CH₂)₂CH₂OCH₃, —CH═CHCH₂OCH₃, —(CH₂)₂CH₂OCH₂CH₃, —CH═CHCH₂OCH₂CH₃, —(CH₂)₂CH₂O(CH₂)₂CH₃, —CH═CHCH₂O(CH₂)₂CH₃, —(CH₂)₂CH₂OCH(CH₃)₂, —CH═CHCH₂OCH(CH₃)₂, —(CH₂)₂CH₂O(CH₂)₃CH₃, —CH═CHCH₂O(CH₂)₃CH₃, a carbon linkage to an aromatic monomer or an aromatic oligomer.

Statement 2* The composition of statement 1*, wherein the one or more aromatic compound active ingredient are dimers, trimers and/or tetramers.

Statement 3* The composition of any one of the statements 1* to 2*, wherein the one or more aromatic compound active ingredient are at a concentration of 0.05 to 20 mg/ml, preferably 0.2 to 10 mg/ml, preferably 0.5 to 5 mg/ml, more preferably 0.8 to 1.2 mg/ml and most preferably 1 mg/mL or in case of a dry composition at 0.5 to 30 wt % by dry weight of aromatic compounds, preferably 1 to 20 wt % by dry weight of aromatic compounds, more preferably 2 to 10 wt % by dry weight of aromatic compounds.

Statement 4* The composition of any one of the statements 1* to 3*, further comprising a further plant defense elicitor such as alginate, hexokinase, laminarin, sodium silicate, silicon, oligo-galacturonan, cellodextrin and/or chito-oligosaccharide or a plant defense elicitor selected from the group consisting of pectin fragment, oligogalacturonide, cellobiose, xyloglucan, non-branched P-1,3-glucan, chitin fragment, arabinose, arabinan, rhamnose, homogalacturonan, rhamnogalacturonan I and II, xylogalacturonan, starch, and combinations thereof.

Statement 5* The composition of any one of the statements 1* to 4*, comprising a) The plant defense elicitor of statement 1* and b) a fungicide, e.g., a fungicide selected from the group consisting of selected from the group comprising: phosphonates, benzamides, carbamates, dithiocarbamates, phthalimides, triazoles, quinolines, sulfur, and cyanoimidazoles or a fungicide selected from the group consisting of 2,6-dichloro-N-[3-chloro-5-(trifluoromethyl)-2-pyridinylmethyl]benzamide; propyl 3-(dimethylamino) propylcarbamate hydrochloride; (2RS,3SR)-1-[3-(2-chlorophenyl)-2,3-epoxy-2-(4-fluorophenyl)propyl]-1H-1,2,4-triazole; 5,7-dichloro-4-quinolyl 4-fluorophenyl ether; sulfur; 4-chloro-2-cyano-N,N-dimethyl-5-(4-methylphenyl)-1H-imidazole-1-sulfonamide; N-(trichloromethylthio)phthalimide; manganese ethylenebis(dithiocarbamate) (polymeric) complex with zinc salt and methyl (E)-methoxyimino-{(E)-α-[1-(α,α,α-trifluoro-m-tolyl)ethylideneaminooxy]-o-tolyl}acetate; or a combination thereof.

Statement 6* The composition of any one of the statements 1* to 5*, comprising a) The plant defense elicitor of statement 1* and b) a salt and/or sugar.

Statement 7* The composition of any one of the statements 1* to 6*, further comprising a co-formulant selected from the group comprising: detergents, emulsifiers, dispersing agents, anti-foaming agents, penetration enhancers, humectants, wetting agents of ionic or non-ionic type, anti-freeze agents, preservative agents, absorbent agents, thickeners, buffers, sticker agents, diluents or a mixture thereof, preferably a surfactant selected from the group comprising: detergents, emulsifiers, dispersing agents, anti-foaming agents, penetration enhancers, humectants or wetting agents of ionic or non-ionic type, or a mixture thereof.

Statement 8* The composition of any one of the statements 1* to 7*, further comprising a surfactant comprising one or more of the following components: castor oil ethoxylate, rapeseed methyl ester, alkyl phosphates, tributyl phosphate, tripropyl phosphate, naphthalenesulphonic acid salts, a combination of organic sulfonate and 2-methylpentane-2,4-diol, alkylpolyglucoside, siloxanes derivates, alkylsulfonates, polycarboxylates, lignosulfonates, alkoxylated triglycerides, fatty amines polymers, dioctylsulfosuccinates or polyoxyethylene (20) sorbitan monolaurate, preferably Cis-castor-oil-ethoxylate, a combination of organic sulfonate and 2-methylpentane-2,4-diol, or polyoxyethylene (20) sorbitan monolaurate.

Statement 9* The composition of any one of the statements 1* to 8*, wherein the composition has a pH in the range of 4 to 10, preferably in the range of 5 to 8.

Statement 10* The composition of any one of the statements 1* to 9*, wherein the ingredients have in origin a pH in the range of 4.0 to 6.0.

Statement 11* Use of the composition according to any of statements 1* to 10* in agricultural applications or to protect plants against plant pests.

Statement 12* Use according to statement 11*, wherein the plant pests are selected from the group comprising: phytopestic fungi, oomycetes, bacteria, viruses, nematodes and insects.

Statement 13* Use of the composition according to any of statements 1* to 10*, to enhance the efficacy of the fungicide in the composition, or to stimulate the plant immune system.

Statement 14* A method for protecting plants against plant pests, comprising applying an effective and substantially non-phytotoxic amount of the composition according to any of statements 1* to 10* to the plants.

Statement 15* The method according to statement 12*, wherein the plant pests are selected from the group comprising: fungi, oomycetes, bacteria, viruses, nematodes and insects.

Statement 16* The method according to any of statements 14* to 114*, wherein the composition is applied before harvest or post-harvest to the whole plant, the leaves, the flowers, fruits, seeds, seedlings or seedlings pricking out, propagation material such as tubers or rhizomes, plants pricking out, and/or to the soil or inert substrate wherein the plant is growing or in which it is desired to grow, by spraying, drenching, soaking, dipping, injection or administration through fertilizing or irrigation systems.

Statement 17* The method according to anyone of statements 14* to 16*, wherein the plant is selected from the group comprising: cotton, flax, vine, fruit, vegetable, major horticultural and forest crops such as: rosaceae sp., ribesioidae sp., juglandaceae sp., betulaceae sp., anacardiaceae sp., fagaceae sp., moraceae sp., oleaceae sp., actinidaceae sp., lauraceae sp., musaceae sp., rubiaceae sp., theaceae sp., sterculiceae sp., rutaceae sp., solanaceae sp., vitaceae sp., liliaceae sp., asteraceae sp., umbelliferae sp., cruciferae sp., chenopodiaceae sp., cucurbitaceae sp., papilionaceae sp., such as graminae sp., fabacae sp.

The present application also provides aspects and embodiments as set forth in the following statements (1″-31″):

Statement 1″ A plant defense elicitor, characterized in that, the resistance-inducing or defense-eliciting active ingredient thereof comprises an effective dose of an aromatic compounds with a DP of 2 to 8 (preferably 2-4) or a molecular mass between 180 g/mol to 1800 g/mol, preferably between 230 g/mol to 1000 g/mol, and yet more preferably between 230 g/mol to 650 g/mol (FIG. 2A-2D), and is an aromatic compound and wherein and/or I) the aromatic compounds comprise at least one of the aromatic compounds from the formulae

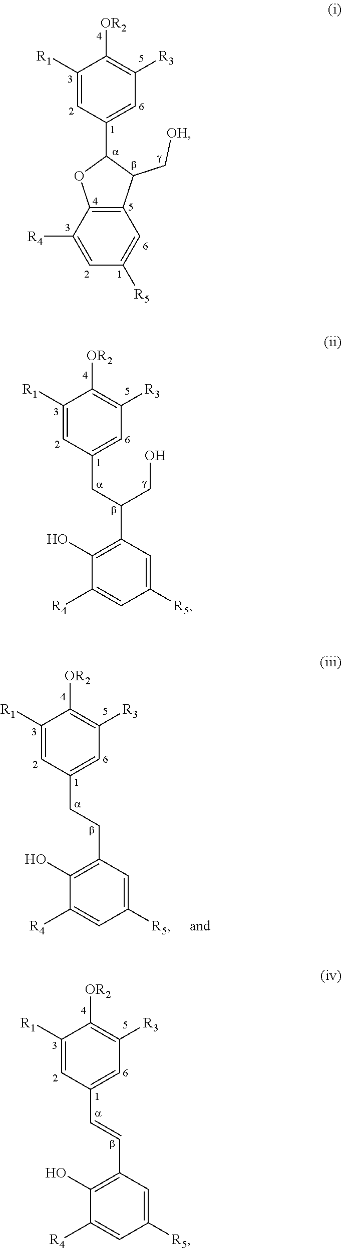

and
wherein each R₁, R₃, and R₄ is independently chosen from —H, —OH, O—CH₃, a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or aromatic oligomer, or a carbon-oxygen linkage to an aromatic monomer or aromatic oligomer,
wherein R₂ is —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer, or a carbon-oxygen linkage to an aromatic monomer or aromatic oligomer,
wherein R₅ is selected from —H, a β-O-4 linkage to an aromatic monomer or aromatic oligomer, a β-5 linkage to an aromatic monomer or aromatic oligomer, a β-β linkage to an aromatic monomer or aromatic oligomer, a β-1 linkage to an aromatic monomer or aromatic oligomer, an end-unit selected from CH₃, —CH₂CH₃, —(CH₂)₂CH₃, —CH₂CH═CH₂, —CH═CHCH₃, —(CH₂)₂CH₂OH, —(CH₂)₂CHO, —CH═CHCH₂OH, —(CH₂)₂CH₂OCH₃, —CH═CHCH₂OCH₃, —(CH₂)₂CH₂OCH₂CH₃, —CH═CHCH₂OCH₂CH₃, —(CH₂)₂CH₂O(CH₂)₂CH₃, —CH═CHCH₂O(CH₂)₂CH₃, —(CH₂)₂CH₂OCH(CH₃)₂, —CH═CHCH₂OCH(CH₃)₂, —(CH₂)₂CH₂O(CH₂)₃CH₃, —CH═CHCH₂O(CH₂)₃CH₃,
and/or II) the aromatic compounds comprise at least one aromatic compound selected from the formulae

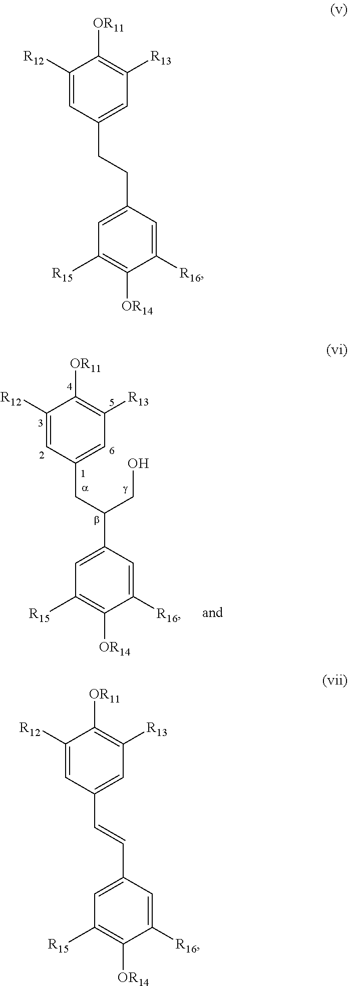

and
wherein each R₁₂, R₁₃, R₁₅ and R₁₆ is independently chosen from —H, —OH, O—CH₃, a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or aromatic oligomer, or a carbon-oxygen linkage to an aromatic monomer or aromatic oligomer,
wherein R₁₁ and R₁₄ is independently chosen from —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer or a carbon-oxygen linkage to an aromatic monomer or aromatic oligomer, and/or III) wherein at the aromatic compounds comprise at least one aromatic compound selected from the formula

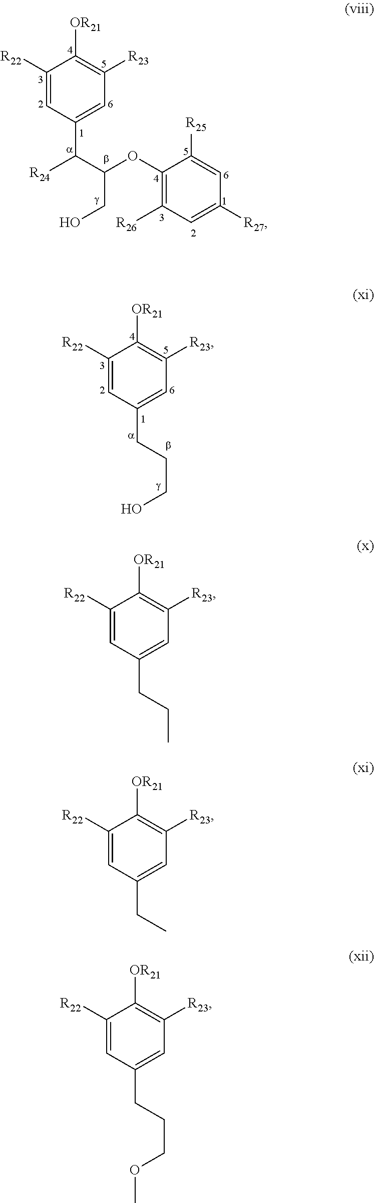

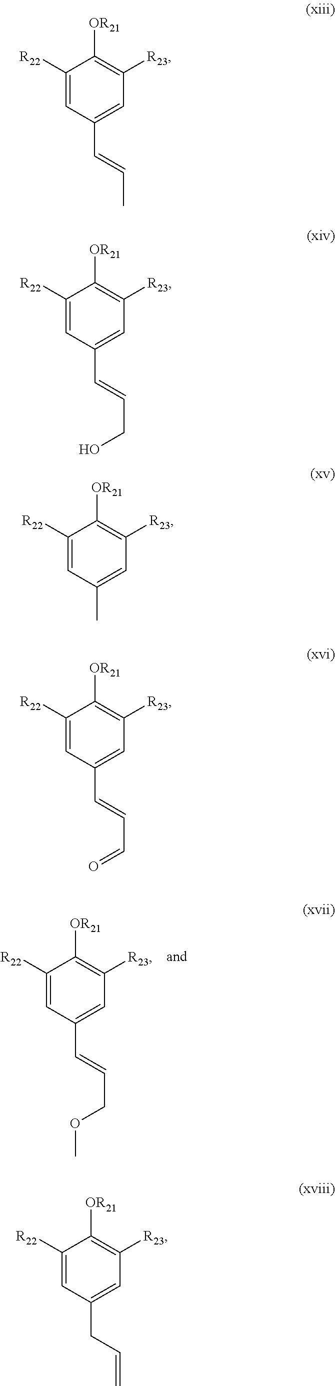

each with at least one linkage to an aromatic monomer or aromatic oligomer and
wherein each R₂₂, R₂₃, R₂₅ and R₂₆ is independently chosen from —H, —OH, O—CH₃, a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or an aromatic oligomer, or a 4 carbon-oxygen linkage to an aromatic monomer or aromatic oligomer,
wherein R₂₁ is independently chosen from —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer or a carbon-oxygen linkage to an aromatic monomer or aromatic oligomer,
wherein R₂₄ is independently chosen from —H, OH, or —O-Alkyl wherein the alkyl group is derived from the alcohol solvent of the process,
wherein R₂₇ is independently chosen from —H, a β-O-4 linkage to an aromatic monomer or aromatic oligomer, a β-5 linkage to an aromatic monomer or aromatic oligomer, a β-β linkage to an aromatic monomer or aromatic oligomer, a β-1 linkage to an aromatic monomer or aromatic oligomer, end-unit selected from CH₃, —CH₂CH₃, —(CH₂)₂CH₃, —CH₂CH═CH₂, —CH═CHCH₃, —(CH₂)₂CH₂OH, —(CH₂)₂CHO, —CH═CHCH₂OH, —(CH₂)₂CH₂OCH₃, —CH═CHCH₂OCH₃, —(CH₂)₂CH₂OCH₂CH₃, —CH═CHCH₂OCH₂CH₃, —(CH₂)₂CH₂O(CH₂)₂CH₃, —CH═CHCH₂O(CH₂)₂CH₃, —(CH₂)₂CH₂OCH(CH₃)₂, —CH═CHCH₂OCH(CH₃)₂, —(CH₂)₂CH₂O(CH₂)₃CH₃, —CH═CHCH₂O(CH₂)₃CH₃, a carbon linkage to an aromatic monomer or an aromatic oligomer.

Statement 2″: The plant defense elicitor of statement 1″, characterized in that, the resistance-inducing or defense-eliciting active ingredient is extracted or derived from depolymerized lignin.

Statement 3″: The plant elicitor according to statement 1″ or 2″, characterized in that the resistance-inducing or defense-eliciting active ingredients are diphenolics, triphenolics and or tetraphenolics comprising compounds wherein the aromatic compounds have two, three or four aromatic groups or a DP of 2, 3 or 4, respectively.

Statement 4″: The plant defense elicitor of any one of the statements 1″ to 3″, wherein the resistance-inducing or defense-eliciting active ingredients are diphenolics comprising compounds wherein the aromatic compounds have 2 aromatic groups or a DP of 2.

Statement 5″ The plant defense elicitor, with in a dry composition a content of at least 0.5% by dry weight of the resistance-inducing or defense-eliciting active ingredient according to any one of the statements 1″ to 4″.

Statement 6″ The plant defense elicitor, with in a dry composition a content of at least 2% by dry weight of the resistance-inducing or defense-eliciting active ingredient according to any one of the statements 1″ to 4″.

Statement 7″ The plant defense elicitor, with in a dry composition a content of at least 5% the resistance-inducing or defense-eliciting active ingredient according to any one of the statements 1″ to 4″.

Statement 8″ The plant pest resistance inducer or plant defense elicitor according to any one of the statements 1″ to 6″, wherein the resistance-inducing or defense-eliciting active ingredient extracted from depolymerized lignin is from a lignocellulose or lignin source.

Statement 9″ The plant defense elicitor of any one of the statements 1″ to 8″, comprising further a repolymerization inhibitor of the group of a carbocation scavenger, aromatic scavengers and a polyhydric alcohol or a combination thereof.

Statement 10″ The plant defense elicitor of any one of the statements 1″ to 9″, comprising a repolymerization inhibitor wherein the repolymerization inhibitor is a compound of the group consisting of citric acid, salicylic acid, 2-Naphthol, phenolic acids (e.g., vanillic acid, syringic acid), ethylene glycol, glycerol, Mannitol (C6H14O6), Sorbitol (C6H14O6), Xylitol (C5H12O5), Erythritol, Maltitol (C12H24O11).

Statement 11″ The plant defense elicitor of any one of the statements 1″ to 9″, with a stabilizing agents of the group consisting of 1,4-butanediol, 2-hydroxy-1-naphthoic acid, 2-naphthol, 2-naphthol-7-sulfonat, 2-naththol, 3-hydroxy-2-naphthoic acid, 4-hydroxybenzoic acid, 6-hydroxy-2-naphthoic acid, ascorbic acid, bovine serum albumin, citric acid, salicylic acid, cysteine, dimethylphloroglucinol, histidine, mannitol, o-dihydroxybenzene, p-benzenediol, phloroglucinol, resorcinol, soy protein isolate, syringic acid and vanillic acid so to prevent repolymerization.

Statement 12″ The plant defense elicitor of any one of the statements 1″ to 11″, further comprising a surfactant.

Statement 13″ The plant defense elicitor of statement 12″, wherein the surfactant is from the group of a lecithin (such as lyso-lecithin), a cyclic lipopeptides biosurfactant (such as surfactin or pseudofactin), a polar aprotic solvent (such as dimethyl sulfoxide, DMSO or a natural polar aprotic solvent of the group consisting of δ-valerolactone (DVL), Cyrene, (dihydrolevoglucosenone); ethyl acetate, methyl lactate, acetone (dimethyl ketone), limonene oxide) and a surface-active agent such as surface active glycolipid or sophorolipid.

Statement 14″ The plant defense elicitor of any one of the statements 1″ to 13″, further comprising a metal such as zinc and/or copper.

Statement 15″ The plant defense elicitor of any one of the statements 1 to 14″, wherein the phenolics that are derived from depolymerized lignin are the reaction product of a reductive catalytic fractionation (RCF) of the lignin source.

Statement 16″ The plant defense elicitor of any one of the statements 1″ to 15″, wherein the phenolics that are derived from depolymerized lignin are the reaction product of a non-catalytic thermosolvolytic de-polymerization and fractioning of the lignin source.

Statement 17″ The plant defense elicitor of any one of the statements 1″ to 15″, wherein the phenolics that are derived from depolymerized lignin and wherein the depolymerized lignin is the reaction product of a solvolytic lignin depolymerization of the lignin source.

Statement 18″ The plant defense elicitor of any one of the statements 1″ to 17″, for promoting induced systemic resistance in a plant or for inducing latent host defenses in a plant by priming of the intrinsic disease resistance mechanisms of a plant.

Statement 19″ The plant defense elicitor of any one of the statements 1″ to 18″, characterized in that it has a pH in the range of 4 to 10, preferably in the range of 5 to 8.

Statement 20″ The plant defense elicitor of any one of the statements 1″ to 18″, wherein the aromatic compounds are in origin an oil with a pH in the range of 4.0 to 6.0 in origin.

Statement 21″ Use of the plant defense elicitor of any one of the statements 1″ to 20″, on a plant or parts thereof wherein the latent host defenses are activated preventive to or in the event of an attack by a phytopathogenic pathogen or pest or by an abiotic stressor.

Statement 22″ Use of the plant defense elicitor of any one of the statements 1″ to 20″, on a plant or parts thereof wherein the latent host defenses are activated preventive to or in the event of an attack by a phytopathogenic pathogen or pest wherein the phytopathogenic pathogen or pest is selected from the group of a fungi, a bacteria, and an insect.

Statement 23″ Use of the plant defense elicitor of any one of the statements 1″ to 20″, on a plant or parts thereof wherein the latent host defenses are activated preventive to or in the event of an attack by a phytopathogenic of the group consisting of fungi, bacteria, viruses, viroids, mycoplasma-like organisms, protozoa, insects, acari, and nematodes.

Statement 24″ Use of the plant defense elicitor of any one of the statements 1″ to 20″, on a plant or parts thereof wherein the latent host defenses are activated preventive to or in the event of abiotic stress.

Statement 25″ Use of the plant defense elicitor of any one of the statements 1″ to 20″, on a plant or parts thereof to prime the intrinsic resistance mechanisms of a plant for stronger or faster induced plant defense preventive to or in the event of an attack by a phytopathogenic pathogen or pest or of abiotic stress, as compared to control or other plant defense inducers.

Statement 26″ Use of the plant defense elicitor of any one of the statements 1″ to 20″, wherein the plant resistance inducer is a foliar spray agent.

Statement 25″ Use of the plant defense elicitor of any one of the statements 1″ to 20″, wherein the plant resistance inducer is a root drench.

Statement 28″ A method for promoting induced systemic resistance in a plant, for inducing latent host defenses of a plant, or for priming the intrinsic resistance mechanisms in a plant, comprising applying to a plant or a plant part, a composition comprising the plant defense elicitor of any one of the statements 1 to 20.

Statement 29″ A method for promoting induced systemic resistance of a plant, for inducing latent host defenses of a plant or for priming the intrinsic resistance mechanisms in a plant, comprising by spraying on the plant or contacting the roots of the plant with the composition comprising the plant defense elicitor of any one of the statements 1″ to 20″.

Statement 30″ Any one of the statements according to 1″ to 29″, wherein lignin-derived diphenolics are the reaction product of the lignin depolymerization or 1) by a reductive catalytic fractionation (RCF) (FIG. 1A) of the lignin source with a heterogenous metal catalyst, including but not limited to Ru, Pd and Ni, on a support in an organic solvent or an organic solvent water mixture in a temperature range of 100° C. to 300° C., preferably 150° C. to 270° C. and most preferably 200° C. to 250° C. and containing a hydrogen donor, including but not limited to H₂ or 2) by a non-catalytic thermosolvolytic de-polymerization (FIG. 1B) of the lignin source in an organic solvent or an organic solvent water mixture in a temperature range of 100° C. to 300° C., preferably 150° C. to 270° C. and most preferably 200° C. to 250° C. and under an inert atmosphere.

Statement 31″ A method for controlling a plant disease comprising treating a plant with a plant defense elicitor according to any one of the statements 1″ to 29″ comprising 0.05 to 20 mg/ml, preferably 0.2 to 10 mg/ml, preferably 0.5 to 5 mg/ml, more preferably 0.8 to 1.2 mg/ml and most preferably 1 mg/mL of active ingredient aromatic compounds or in case of a dry composition 0.5 to 30 wt % by dry weight of aromatic compounds, preferably 1 to 20 wt % by dry weight of aromatic compounds, more preferably 2 to 10 wt % by dry weight of resistance-inducing or defense-eliciting active ingredient.

The disclosure is further described with the aid of the following illustrative EXAMPLES.

### EXAMPLES

### Example 1: Preparation of Depolymerized Lignin Via Reductive Catalytic Fractionation (RCF)

FIG. 1A shows typical input and output of an RCF reaction that can be performed under varying conditions with use of a heterogenous catalyst and under a reductive environment or containing a hydrogen donor as detailed below. The RCF experiment was performed in a 2 L stainless steel batch reactor (Parr Instruments & Co.). 150 g lignocellulose biomass (e.g., pine or spruce, further referred to as “pine” or poplar) was loaded into the reactor, together with 15.0 g Pd/C or 15.0 g Ru/C and 800 mL methanol. Subsequently, the reactor was sealed, flushed three times with N₂ (10 bar) and then pressurized with H₂ (30 bar at room temperature). Next, the reaction mixture was stirred (750 rpm) and simultaneously heated to 235° C. (˜30 minutes heating time). After the reaction time of 3 hours, the reactor was cooled and depressurized at room temperature. The reactor contents were quantitatively collected by washing the reactor with ethanol.

The solid pulp was separated by filtration and washed thoroughly with ethanol. Next, the resulting filtrate was evaporated and a brown oil was obtained, which was subjected to a threefold liquid-liquid extraction using ethyl acetate and water in order to remove extracted sugars. Lastly, the ethyl acetate-extracted phase was dried to obtain the lignin oil. The resulting oils are named with reference to the biomass and catalyst used during the RCF (e.g., pineRuOil).

### Example 2: Preparation of Depolymerized Lignin Via Non-Catalytic Thermosolvolytic Fractioning

FIG. 1B shows typical input and output streams of a non-catalytic thermos-solvolytic fractioning where no catalyst is used. The non-catalytic thermosolvolytic fractioning experiment was performed in an identical set-up to the RCF and biomass feedstock with the difference of excluding the redox catalyst and under inert atmosphere, pressurizing with N2 (to 30 bar at room temperature). All other procedures were kept identical. The resulting lignin oils are named with reference to the biomass used during the non-catalytic thermosolvolytic fractioning (e.g., pine non-catalytic thermosolvolytic fractioning oils or PineOil).

### Example 3: Fractionation by Sequential Liquid-Liquid Solvent Extraction

FIG. 1C shows fractionation of lignin oil through liquid-liquid extraction. An initial fractionation step involved the lignin oil from RCF or non-catalytic thermosolvolytic fractioning and heptane solvent at a 1:5 ratio (g/mL) with threefold extraction at 80° C. for 0.5 hours. After each extraction, the soluble fraction (H100 liquid, HeptFra, H100L) was separated from the residual fraction (H100 residue, HeptRes, H100R), as shown in FIG. 1C, and all solvents of both fractions were removed by rotary evaporator and dried at 80° C. in an oven. FIG. 1C also shows subsequent fractionation steps on the heptane residue, if performed, using a mixture of heptane and ethyl acetate in the v:v ratio of 80% heptane/20% ethyl acetate. Specific fractions are denoted based on the origin of the lignin oil and the solvent composition that was used to obtain the fraction: for example, PineRu H80E20 residue (PineRuH80E20R).

### Example 4: GPC Analysis

FIG. 2 shows the distribution of the molar mass of the refined lignin oils and fractions obtained from various CF reactions with different feedstocks and catalyst (poplar and Ru/C in FIG. 2A, poplar and Pd/C in FIG. 2B, pine and Ru/C in FIG. 2C, pine and Pd/C in FIG. 2D and pine without catalyst in FIG. 2E), as investigated using gel permeation chromatography—size exclusion (GPC-SEC). Therefore, a lignin sample was solubilized in THF (5 mg mL⁻¹) and subsequently filtered with a 0.2 μm PTFE membrane to remove any particulate matter to prevent plugging of the column. GPC-SEC analyses were performed at 40° C. on a Waters E2695 equipped with a PL-Gel 3 μm Mixed-E column with at length of 300 mm, using THE as a solvent with a flow of 1 mL min⁻¹. The detection was UV based at a wavelength of 280 nm. Calibration was based on calibration with commercial polystyrene standards of Agilent.

### Example 5: Elicitor Treatment on A. Thaliana-H. arabidopsidis Pathogen System

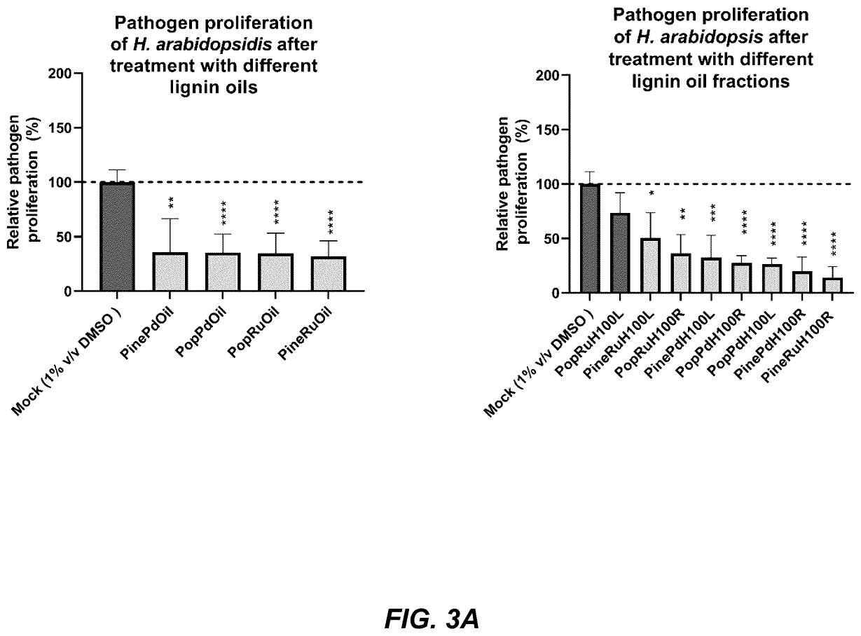

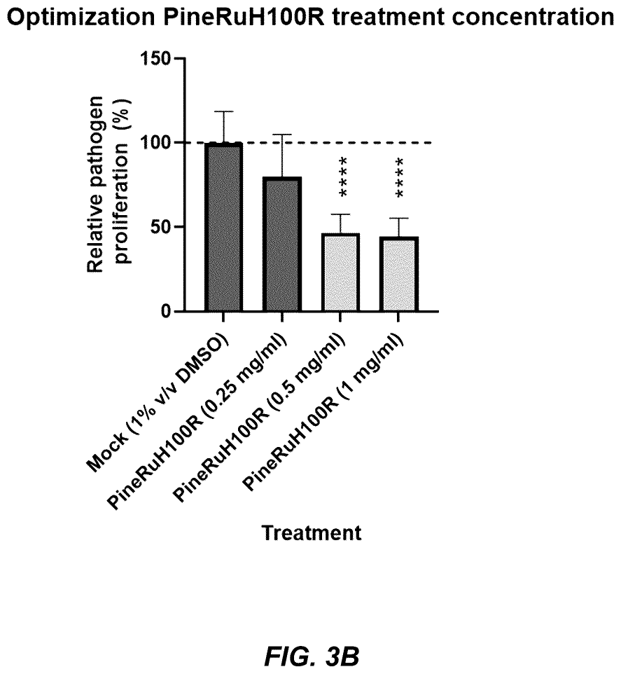

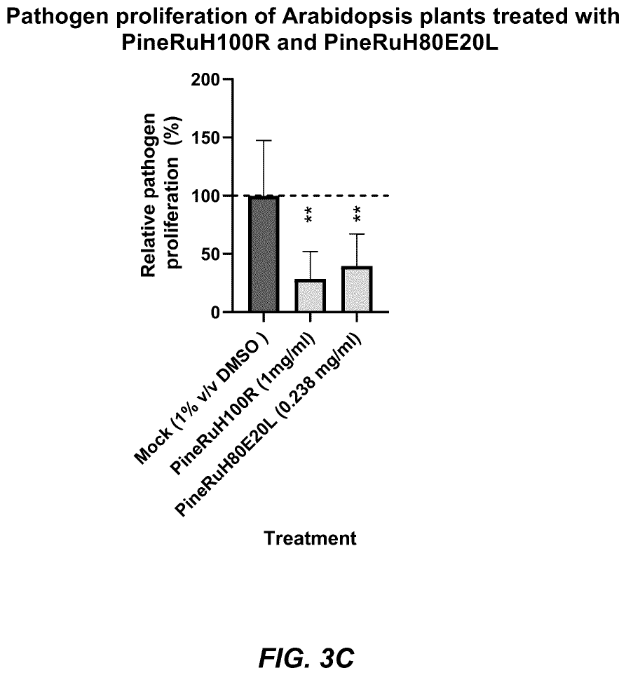

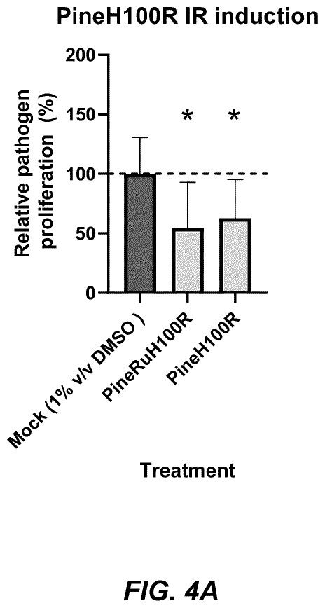

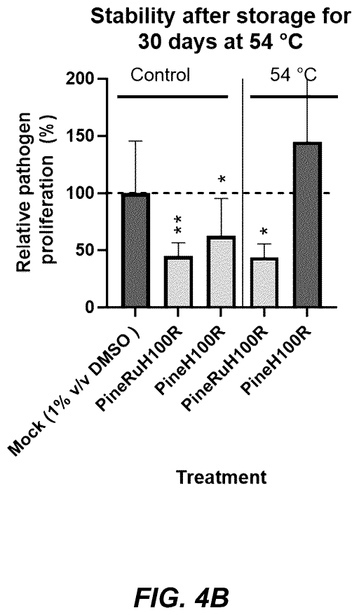

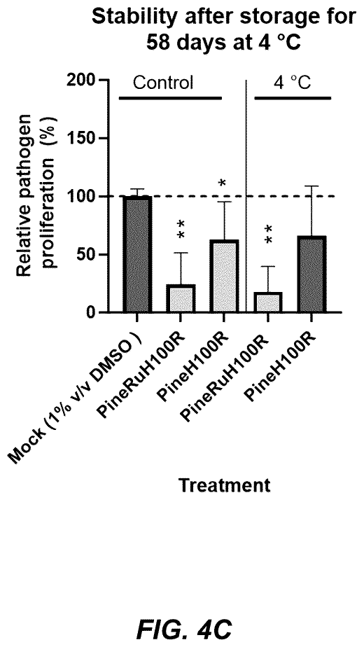

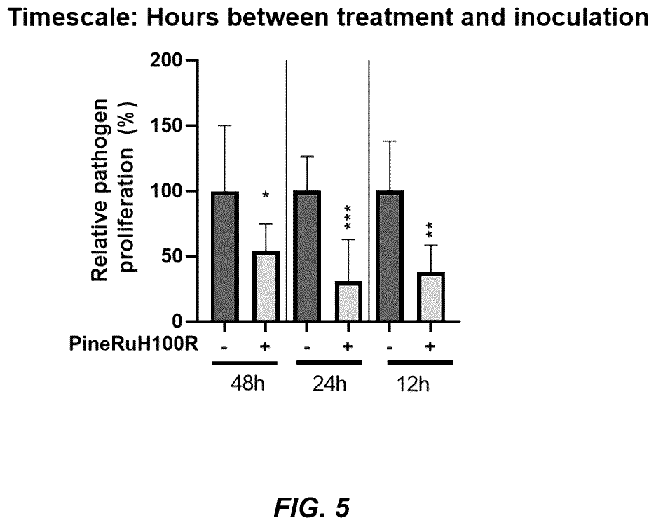

FIG. 3A depicts the A. thaliana-H. arabidopsidis disease assay A. thaliana (ecotype: Columbia-0) plants were seeded in little pots (approximately 40 seeds/pot). Five to seven days after sowing, seedlings were selected so that 20 well-developed, freestanding seedlings were retained in each pot. Eight days after sowing, the plants were treated with mock (1% v/v DMSO), any of the depolymerized lignin fractions (varying concentrations) (1 mg/ml) fraction by spraying the leaves with compound solution until run-off (˜20 ml was used per 8 biological replicates). Twenty-four hours after treatment, the leaves of the plants were inoculated by spraying them with a H. arabidopsidis (per 23 pots, 15 ml of spore solution) Noks1 spore suspension of 4.875-8.125×10⁴ spores/ml in cold dH2O until run-off. Plants were placed in a closed infection box with high humidity and the infection box was placed in a growth chamber under controlled conditions (16° C., 70% humidity, 12-hour day-night cycle, light intensity of approximately 100 μmol/m2s). Three days post-infection, leaves of 15 plants per pot were taken, transferred to an Eppendorf tube containing 250 μl dH₂O and stored at −80° C. and used for genomic DNA extraction with qPCR to determine relative pathogen proliferation.

### Example 6: Genomic DNA Extraction and qPCR

For isolation of plant genomic DNA (gDNA), plant samples were grinded using the Precellys™ 24-tissue homogenizer at 6000 rpm for 10 seconds. After homogenization, 400 μl of Edwards buffer (200 mM Tris-HCl pH 7.5, 250 mM NaCl, 25 mM EDTA, and 0.5% (v/v) SDS) was added to each sample. The samples were vortexed and incubated at 55° C. for 15 minutes. After incubation, samples were centrifuged for 2 minutes at 13000 rpm. Subsequently, 20 μl of the supernatant was transferred to a new, sterile 1.5 ml Eppendorf tube and an equal amount of isopropanol was added to precipitate the gDNA. The mixture was incubated for 10 minutes at RT, after which it was centrifuged for 10 minutes at 13000 rpm. The supernatant was discarded and the pellet was washed with 70% ethanol. After another centrifugation round of 5 minutes at 13000 rpm, the ethanol was removed and the pellet was dried for one hour at 37° C. The pellet, containing the isolated gDNA, was resuspended in 40 μl sterile demi water. The concentration of gDNA was determined using the NanoDrop™ One Microvolume UV-Vis Spectrophotometer (Thermo Fisher Scientific, US) by measuring the absorbance at 260 nm. The gDNA samples were diluted to 10 ng/μl and stored at −20° C.

The protocol for pathogen growth quantification via qPCR was as follows. Samples for qPCR were prepared by mixing 2.5 μl gDNA (=25 ng) with 6.125 μl 2×SYBR® Green Mastermix (Thermo Fisher Scientific, US) and 500 nM of the reverse and forward primer each. The final volume was adjusted to 12.5 μl using sterile MILLI-Q® water. The samples were loaded in an MicroAmp™ Fast Optical 96-Well Reaction Plate (Thermo Fischer Scientific, US) which was kept on ice. Post loading, the plate was shortly centrifuged. The qPCR was performed in triplicate for each biological sample on the StepOnePlus™ Real-Time PCR system (Thermo Fisher Scientific, US). After 10 minutes at 95° C., samples were run for 40 cycles of 15 seconds at 95° C., 15 seconds at 57° C., and 15 seconds at 72° C. After each run, melting curves were acquired to check for amplification specificity by heating the samples from 60° C. to 95° C. The Ct values were determined by the included StepOne™ software. For each biological replicate, the relative amount of pathogen gDNA over plant gDNA was calculated using the formula below (adapted from Livak and Schmittgen (2001)).

Relative amount gDNA pathogen/gDNA plant=2−ΔCt=2−(Ct pathogen−Ct plant)

### Primers Used are as in Table 1

### Example 7: Elicitor Treatment on Solanum lycopersicum-Botrytis cinerea Pathogen System

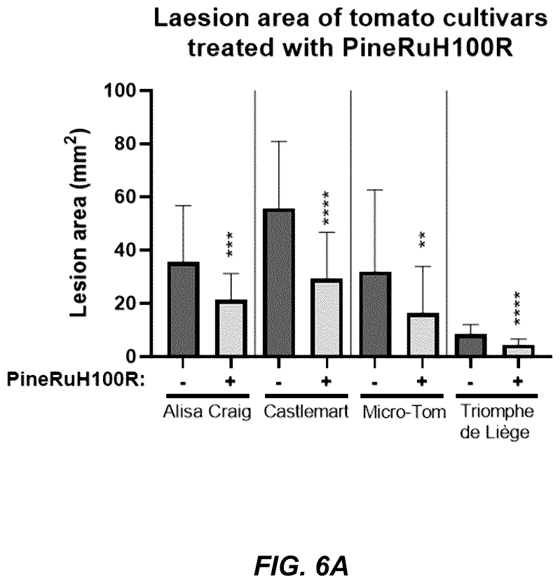

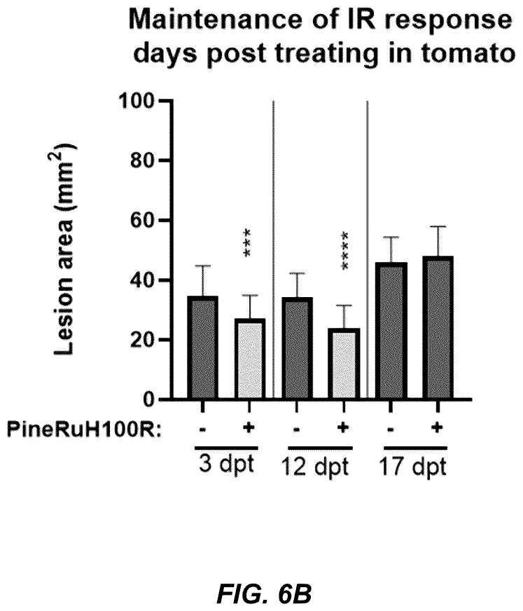

FIG. 6 depicts the hydroponics tomato-B. cinerea assay, with tomato plants grown in a lab-scale hydroponics setup (Araponics Liège, Belgium). The hydroponics tanks were filled with 1.6 1 plant nutrient solution containing the macronutrients MgSO₄·7H₂O (500 mg/l), KH₂PO₄ (270 mg/l), KNO₃ (200 mg/l), K₂SO₄ (100 mg/l), Ca(NO₃)2·4H₂O (500 mg/l), and FeEDTA sodium salt (25 mg/l); and the micronutrients H₃BO₃ (4.1 mg/), MnSO₄·H₂O (3.7 mg/l), CuCl₂·2H₂O (0.2 mg/l), (NH₄)6Mo7O24·4H₂O (0.0825 mg/l with 81.2% MoO3), and ZnSO₄·7H₂O (0.649 mg/l). Tomato cultivar seeds were sown in the hydroponic systems in seed holders (18 seeds/system) containing a solidified 0.65% (w/v) agar in water solution. The hydroponic tanks were covered with plastic lids and placed in the plant growth chamber. One week later, the seeds had germinated so the lids were removed and aeration pumps were installed to aerate the root compartments. After 24 days, 8 plants were selected per system and were treated with plant defense elicitor compounds by spraying the leaves with compound solution until run-off. Treatment with the solvent 1% v/v DMSO was included as mock treatment. Three, twelve and seventeen days after treatment, five leaflets per plant were inoculated with 5 μl droplets of a B. cinerea R16 strain spore suspension of 5×10⁵ spores/ml in ½ potato dextrose broth. The hydroponics tanks were placed inside an infection box, containing a moist mat to obtain high humidity, in the growth chamber. The disease symptoms were quantified by measuring the diameter parallel to the midrib of the developing necrotic lesions at 2 dpi. The lesion area was calculated using the formula below

$$Lesion area = ( Lesion diameter / 2 ) ⋀ 2 ⋆ π$$

### Example 8: Protecting S. lycopersicum Against Insect Feeding—Induction by RCF Lignin of Increased Resistance in Tomato Against Insects

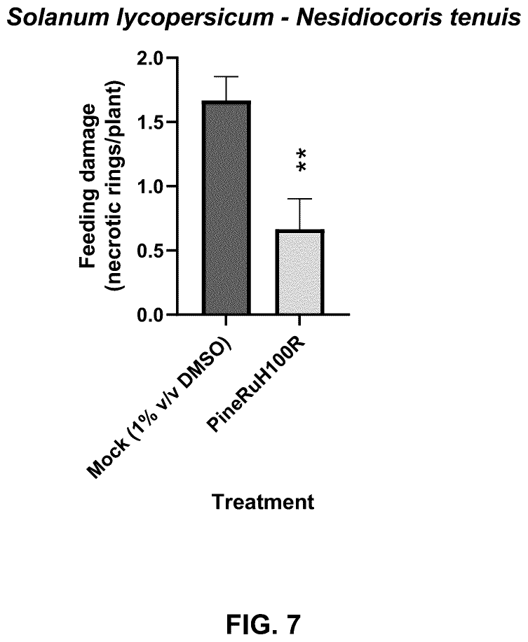

FIG. 7 shows protection of tomato plants (S. lycopersicum) against insect feeding (Nesidiocoris tenuis) by RCF lignin treatment. S. lycopersicum (cultivar: Moneymaker) seeds were disinfected by 5-minute exposure to 20% bleach and subsequently germinated in petri dish covered with moisturized paper. Post germination, seeds were seeded in soil. Thirty-eight days post seeding, plants were treated with mock (1% v/v DMSO) and PineRuH100R (1 mg/ml) by spraying leaves till run-off. Afterwards, plants were placed in air inflated plant cages (60 cm×40 cm×40 cm with mesh size 0.25 mm×0.25 mm, Entomologie-Speciaalzaak Vermandel V.O.F., the Netherlands), which could be accessed by opening the zipper. Three days after initial treatment, the plants received a second treatment 3 hours prior to insect infestation. Plants were infested by introducing one N. tenuis female per plant cage. To force the insects to feed on the plant no additional food or prey were present in the cage. The total number of necrotic rings on leaves and shoots were assessed 7 days after infestation as a measure of feeding damage. Statistical differences were determined by using a student's t test (p<0.05). Experiments were conducted in a fully randomized block design in a climate-controlled greenhouse compartments (T=20° C.+/−4° C., RH=70%, and a 18L:6D photoperiod).

### Example 9: Induction by RCF Lignin of Increased Resistance in Tomato Against Heat Stress

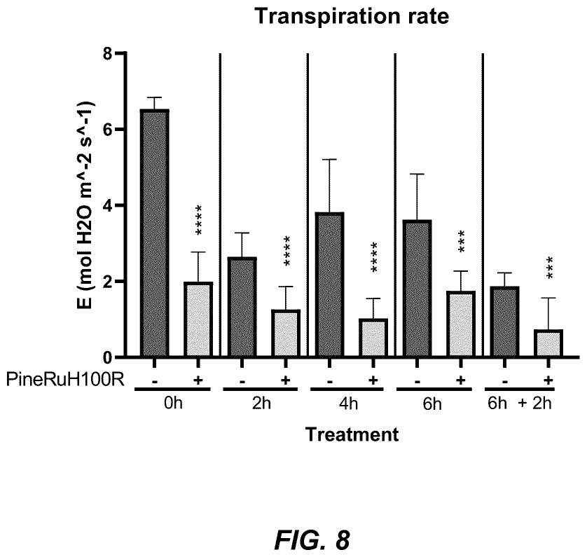

FIG. 8 depicts decreased transpiration rate of tomato plants (S. lycopersicum) by RCF lignin treatment. S. lycopersicum (cultivar: Alisa Craig) were sown in seed holders containing 0.65% (w/v) agar in a water solution placed in a hydroponics tank (Araponics Liege, Belgium) filled with 1.6 L of nutrient solution containing the macronutrients MgSO₄·7H₂O (500 mg/l), KH₂PO₄ (270 mg/l), KNO₃ (200 mg/l), K₂SO₄ (100 mg/l), Ca(NO₃)₂·4H₂O (500 mg/l), and FeEDTA sodium salt (25 mg/l); and the micronutrients H₃BO₃ (4.1 mg/), MnSO₄·H₂O (3.7 mg/l), CuCl₂·2H₂O (0.2 mg/l), (NH₄)6Mo₇O24·4H₂O (0.0825 mg/l with 81.2% MoO₃), and ZnSO₄·7H₂O (0.649 mg/l). After 24 days, S. lycopersicum leaves were sprayed with mock (1% v/v DMSO) or PineRuH100R (1 mg/ml) until run off. Three-days post spraying plants were placed in a heat stress cabinet (38° C.). Determination of transpiration was carried out by clipping the apical side of the youngest fully developed leaf into the LI-COR 600 device at 0 hour, 2 hours, 4 hours, 6 hours and 6 hours+2 hours recovery time post heat-stress initiation.

### Example 10: Improved Survival of Arabidopsis thaliana Under Heat Stress by RCF Lignin Treatment

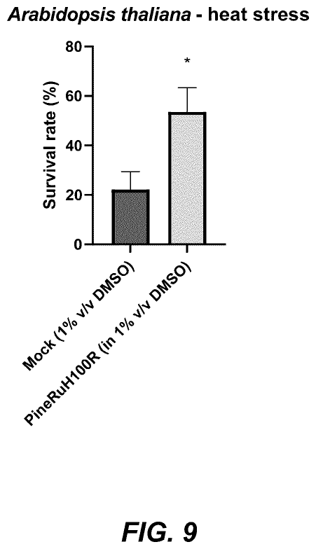

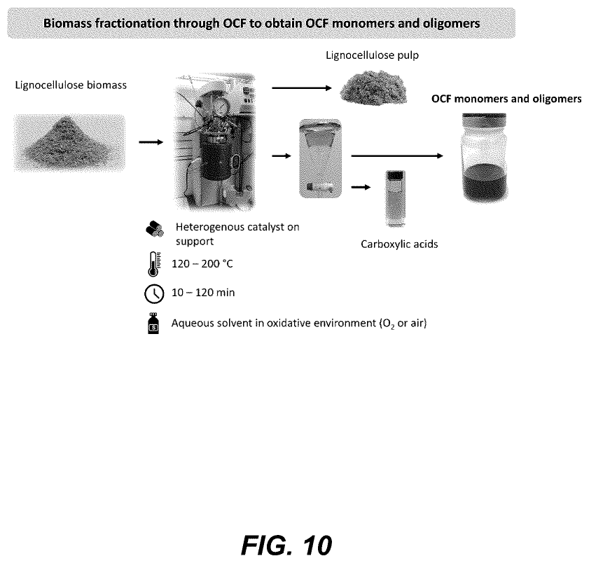

FIG. 9 depicts improved survival rate of A. thaliana under heat stress by RCF lignin treatment. The A. thaliana-heat stress protocol was adapted from Silva-Correi et al. 2014. Briefly, A. thaliana seeds were surface sterilized by 10-minute exposure to 30% bleach. Subsequently, 50 seeds were sown on 1×Murashige and Skoog (MS) plates. Four days post seeding, plants were treated with either mock (1% v/v DMSO) or PineRuH100R (1 mg/ml in 1% v/v DMSO) by submerging seedlings in 5 μl droplets. Three days after, heat stress was imposed by submersion of parafilm-sealed plates into a water bath (45° C.) for 18 min. Following heat treatment, plates were returned to the growth chamber and allowed to recover for six days. Next, seedling survival was assessed by counting seedlings that remained green. Statistical differences were determined by using a student's t test (p<0.05). Plants were grown vertically in climate-controlled growth chambers (T=22° C., RH=70%, and a 12L:12D photoperiod). FIG. 10. demonstrates that the seedling survival is improved in seedlings that were treated with PineRuH100R.

### Example 11: Preparation of Depolymerized Lignin Via Oxidative Catalytic Fractionation (OCF)

FIG. 10 shows typical input and output of an OCF reaction that can be performed under varying conditions with use of a heterogenous catalyst and under oxygen gas (O₂) or air. The OCF experiment was performed in a 50 mL stainless steel batch reactor with an alkali-resisting polyphenylene liner. 0.5 g lignocellulose biomass (birch wood) was loaded into the reactor, together with 1.26 mmol of catalyst (CuO) and 25 mL of NaOH aqueous solution. Subsequently, the reactor was sealed, flushed and then pressurized with O2 (1 MPa at room temperature). Next, the reaction mixture was stirred (150 to 1100 rpm) and simultaneously heated up to 160° C. (˜30 minutes heating time). After the reaction time of maximum 2 hours, the reactor was cooled and depressurized at room temperature.

After reaction, the slurry was centrifuged to separate the solid residue (including the pulp and spent catalyst) and liquid. The obtained liquid was acidified by HCl until pH 2-3. The acidified liquid was extracted with chloroform or ethyl acetate (EtOAc) until the organic phase was colorless. A small amount of NaHCO₃ was then added into the organic phase to neutralize the residual acid. Anhydrous Na₂SO₄ was used to remove water. By evaporation of the organic phase, crude monophenolics and oligomers were obtained, and chloroform/EtOAc can be reused in the extraction step. The water phase was centrifuged to separate acid-insoluble oligomers and the acid (and water)-soluble portion. The acid-insoluble oligomers were washed with deionized water until the pH of the eluent was 7. The acid (and water)-soluble portion was vacuum distilled at 60° C. to remove H₂O and HCl, and solid salts (including NaCl) were obtained. The salts were freeze-dried. The resulting depolymerized lignin mixtures are named OCF 4.1 (OCF monomers) and OCF 4.2 (OCF oligomers).

### Example 12: Elicitor Treatment on A. Thaliana-H. arabidopsidis Pathogen System with OCF Lignin

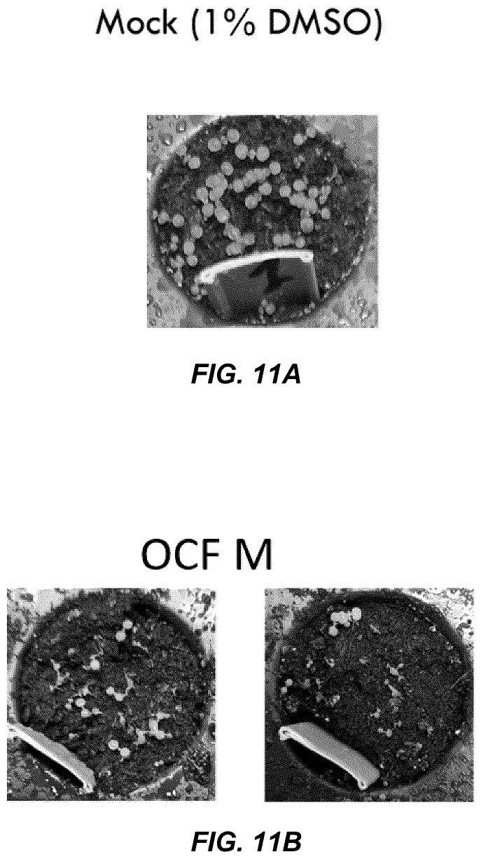

FIG. 11 shows the A. thaliana (ecotype: Columbia-0) plants seedlings in little pots (20 well-developed, freestanding seedlings) treated after eight days after sowing treated with mock (1% v/v DMSO) (FIG. 11A), and depolymerized lignin monomers from OCF (OCF 4.1, 1 mg/ml) (FIG. 11B) fraction by spraying the leaves with compound solution until run-off. The figures clearly show the toxic impact of the monomers from OCF (OCF 4.1) on the plants that resulted in limited growth as compared to the plant seedlings without the treatment.

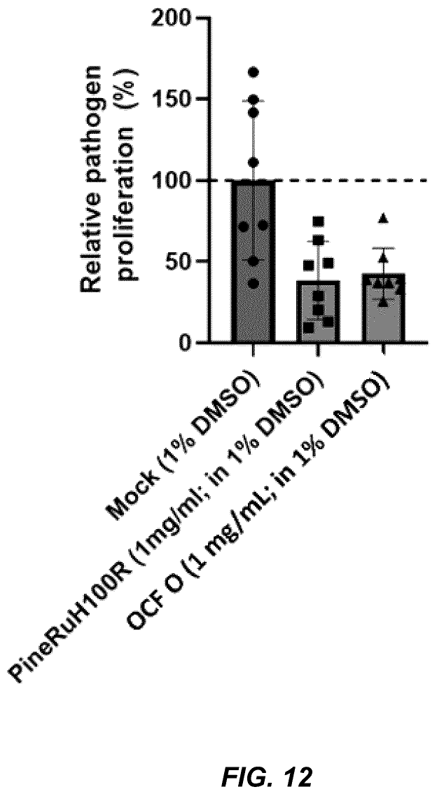

FIG. 12 shows the relative pathogen proliferation of H. arabidopsidis in A. thaliana plants treated with the heptane insoluble fraction from a depolymerized lignin obtained from the catalytic RCF process of pine wood (PineRuH100R) or OCF oligomers (OCF 4.2) obtained from birch wood using a catalytic OCF process demonstrating equal elicitor activity of depolymerized lignin oligomers (DP≥2) from RCF and OCF

TABLE 1
Tables Description

| SEQ ID | Primer | Sequence |
|---|---|---|
| NO: | name | |
| 1 | PFIN760 | 5′-GTG TCG CAC ACT GTA CCC |
| | | ATT TAT-3′ |
| 2 | PFIN761 | 5′-ATC TTC ATC ATG TAG TCG |
| | | GTC AAG T-3′ |
| 3 | PFIN762 | 5′-AAT CAC AGC ACT TGC ACC |
| | | A-3′ |
| 4 | PFIN763 | 5′-GAG GGA AGC AAG AAT GGA |
| | | AC-3′ |

### REFERENCES

Angelova et al. (2006). Elicitation of plants. Biotechnology & Biotechnological Equipment, 20(2), 72-83.
Bektas, Y. (2022). Fytosol, a promising plant defense elicitor, controls early blight (Alternaria solani) disease in the tomato by inducing host resistance-associated gene expression. Horticulturae, 8(6), 484. doi.org/10.3390/horticulturae8060484
Chalal et al. (2015). Sesquiterpene volatile organic compounds (VOCs) are markers of elicitation by sulfated laminarine in grapevine. Frontiers in Plant Science, 6.
Hatami et al. (2019). Nano-elicitation of secondary pharmaceutical metabolites in plant cells: a review. Journal of Medicinal Plants, 3(71), 6-36. doi.org/10.29252/jmp.3.71.6
Maffei, M., Arimura, G., and Mithöfer, A. (2012). Natural elicitors, effectors and modulators of plant responses. Natural Product Reports, 29(11), 1288.
Shinya et al. (2006). Novel β-1,3-, 1,6-oligoglucan elicitor from Alternaria alternata 102 for defense responses in tobacco. Febs Journal, 273(11), 2421-2431.
Sudhamoy, M. (2010). Induction of phenolics, lignin and key defense enzymes in eggplant (Solanum melongena l.) roots in response to elicitors. African Journal of Biotechnology, 9(47), 8038-8047. doi.org/10.5897/ajb10.984.
Wang et al. (2004). Vdnep, an elicitor from Verticillium dahliae, induces cotton plant wilting. Applied and Environmental Microbiology, 70(8), 4989-4995.

What is claimed is:

1. A plant defense elicitor comprising at least one lignin oligomer with a degree of polymerization (DP) of 2 to 8, optionally 2-4.

2. The plant defense elicitor of claim 1, wherein the at least one lignin oligomer has a pH in the range of 5.0 to 8.0.

3. The plant defense elicitor of claim 1, wherein the at least one lignin oligomer comprises:
I) at least one aromatic compound selected from the group of formulae: 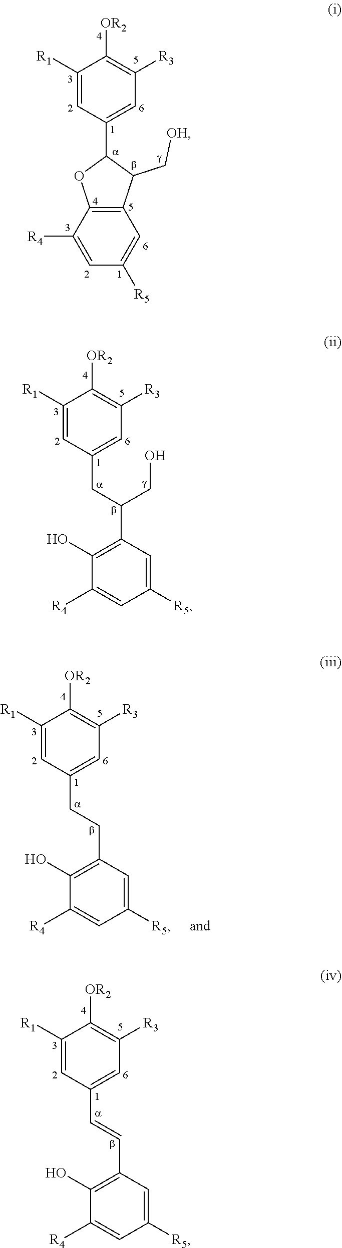
and
wherein each R₁, R₃, and R₄ is independently selected from —H, —OH, O—CH₃, a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or aromatic oligomer, or a carbon-oxygen linkage to an aromatic monomer or aromatic oligomer,
wherein R₂ is —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer, or a carbon-oxygen linkage to an aromatic monomer or aromatic oligomer, and
wherein R₅ is selected from —H, a β-O-4 linkage to an aromatic monomer or aromatic oligomer, a β-5 linkage to an aromatic monomer or aromatic oligomer, a β-β linkage to an aromatic monomer or aromatic oligomer, a β-1 linkage to an aromatic monomer or aromatic oligomer, an end-unit selected from CH₃, —CH₂CH₃, —(CH₂)₂CH₃, —CH₂CH═CH₂, —CH═CHCH₃, —(CH₂)₂CH₂OH, —(CH₂)₂CHO, —CH═CHCH₂OH, —(CH₂)₂CH₂OCH₃, —CH═CHCH₂OCH₃, —(CH₂)₂CH₂OCH₂CH₃, —CH═CHCH₂OCH₂CH₃, —(CH₂)₂CH₂O(CH₂)₂CH₃, —CH═CHCH₂O(CH₂)₂CH₃, —(CH₂)₂CH₂OCH(CH₃)₂, —CH═CHCH₂OCH(CH₃)₂, —(CH₂)₂CH₂O(CH₂)₃CH₃, —CH═CHCH₂O(CH₂)₃CH₃, a carbon linkage to an aromatic monomer or aromatic oligomer;
II) wherein the aromatic compounds comprise at least one aromatic compound selected from the formulae: 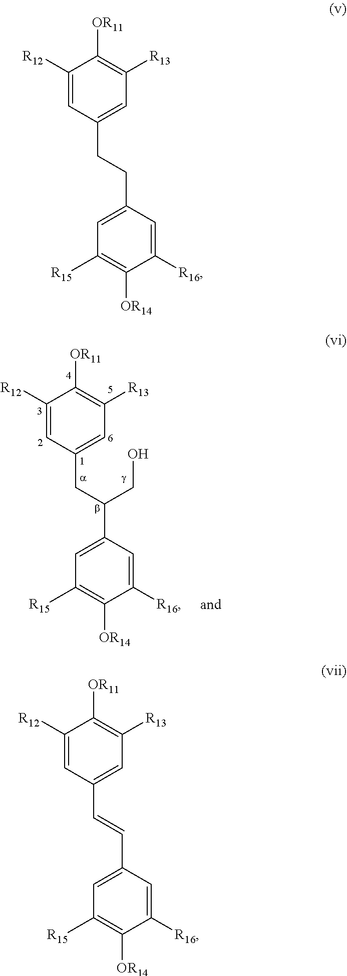
and
wherein each R₁₂, R₁₃, R₁₅ and R₁₆ is independently selected from —H, —OH, O—CH₃, a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or aromatic oligomer, or a carbon-oxygen linkage to an aromatic monomer or aromatic oligomer,
wherein R₁₁ and R₁₄ is independently selected from —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer or a carbon-oxygen linkage to an aromatic monomer or aromatic oligomer;
and/or
III) wherein at the aromatic compounds comprise at least one aromatic compound selected from the group of formula: 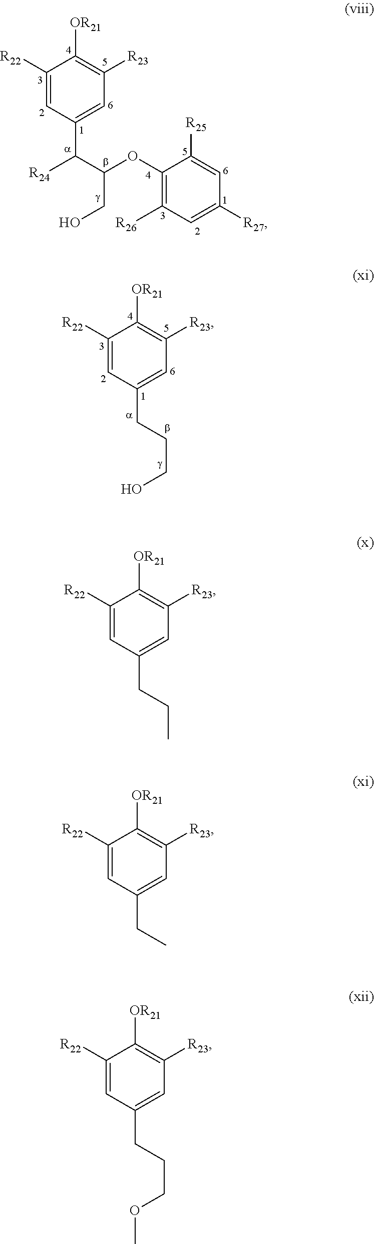 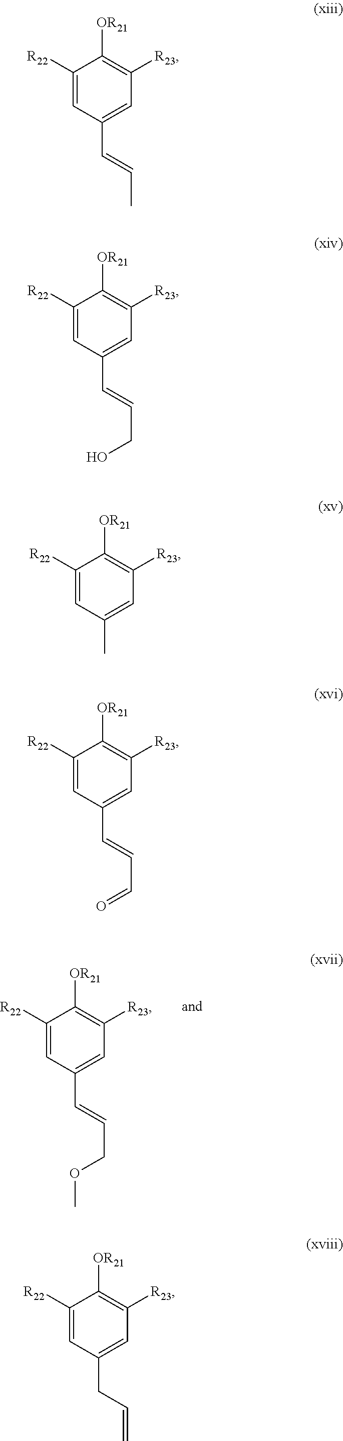
each with at least one linkage to an aromatic monomer or aromatic oligomer, and
wherein R₂₁ is independently selected from —H, a β-O-4 linkage to an aromatic monomer or aromatic oligomer, a 4-O-5 linkage to an aromatic monomer or aromatic oligomer, an α-O-4 linkage to an aromatic monomer or aromatic oligomer or a carbon-oxygen linkage to an aromatic monomer or aromatic oligomer,
wherein each R₂₂ and R₂₃ is dependently selected from —H, —OH, O—CH3 or from a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or an aromatic oligomer, or a carbon-oxygen linkage to an aromatic monomer or aromatic oligomer,
wherein R₂₄ is independently selected from —H, OH, or —O-Alkyl wherein the alkyl group is derived from the alcohol solvent of the process,
wherein each R₂₅ and R₂₆ is independently selected from —H, —OH, O—CH₃, a 4-O-5 linkage to an aromatic monomer or aromatic oligomer, a 5-5 linkage to an aromatic monomer or aromatic oligomer, a β-5 linkage to an aromatic monomer or aromatic oligomer, a carbon linkage to an aromatic monomer or an aromatic oligomer, or a carbon-oxygen linkage to an aromatic monomer or aromatic oligomer,
wherein R₂₇ is independently selected from —H, a β-O-4 linkage to an aromatic monomer or aromatic oligomer, a β-5 linkage to an aromatic monomer or aromatic oligomer, a β-β linkage to an aromatic monomer or aromatic oligomer, a β-1 linkage to an aromatic monomer or aromatic oligomer, end-unit selected from CH₃, —CH₂CH₃, —(CH₂)₂CH₃, —CH₂CH═CH₂, —CH═CHCH₃, —(CH₂)₂CH₂OH, —(CH₂)₂CHO, —CH═CHCH₂OH, —(CH₂)₂CH₂OCH₃, —CH═CHCH₂OCH₃, —(CH₂)₂CH₂OCH₂CH₃, —CH═CHCH₂OCH₂CH₃, —(CH₂)₂CH₂O(CH₂)₂CH₃, —CH═CHCH₂O(CH₂)₂CH₃, —(CH₂)₂CH₂OCH(CH₃)₂, —CH═CHCH₂OCH(CH₃)₂, —(CH₂)₂CH₂O(CH₂)₃CH₃, —CH═CHCH₂O(CH₂)₃CH₃, a carbon linkage to an aromatic monomer or an aromatic oligomer.

4. The plant defense elicitor of claim 1, wherein the at least one lignin oligomers comprises:
I) at least one aromatic compound selected from the formulae: 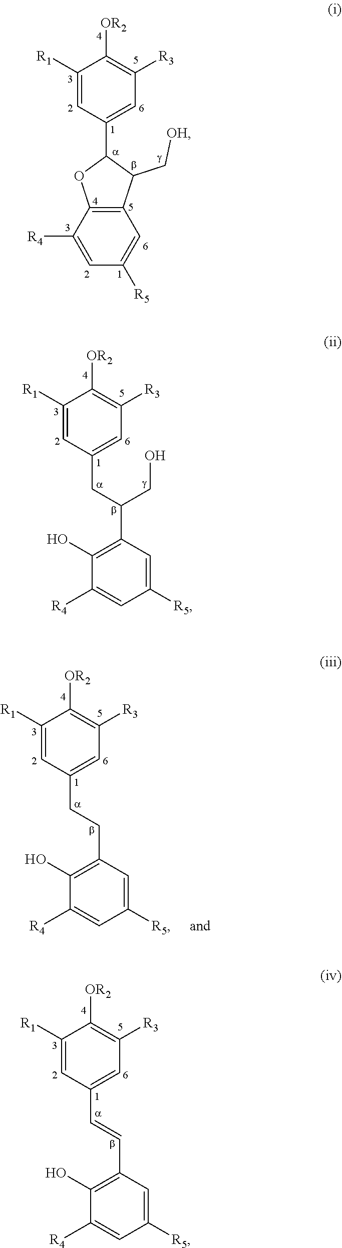
and
wherein each R₁, R₃, and R₄ is independently selected from —H, —OH, O—CH₃,
wherein R₂ is —H,
wherein R₅ is selected from —H, an end-unit selected from CH₃, —CH₂CH₃, —(CH₂)₂CH₃, —CH₂CH═CH₂, —CH═CHCH₃, —(CH₂)₂CH₂OH, —(CH₂)₂CHO, —CH═CHCH₂OH, —(CH₂)₂CH₂OCH₃, —CH═CHCH₂OCH₃, —(CH₂)₂CH₂OCH₂CH₃, —CH═CHCH₂OCH₂CH₃, —(CH₂)₂CH₂O(CH₂)₂CH₃, —CH═CHCH₂O(CH₂)₂CH₃, —(CH₂)₂CH₂OCH(CH₃)₂, —CH═CHCH₂OCH(CH₃)₂, —(CH₂)₂CH₂O(CH₂)₃CH₃, —CH═CHCH₂O(CH₂)₃CH₃;
II) wherein the aromatic compounds comprise at least one aromatic compound selected from the formulae: 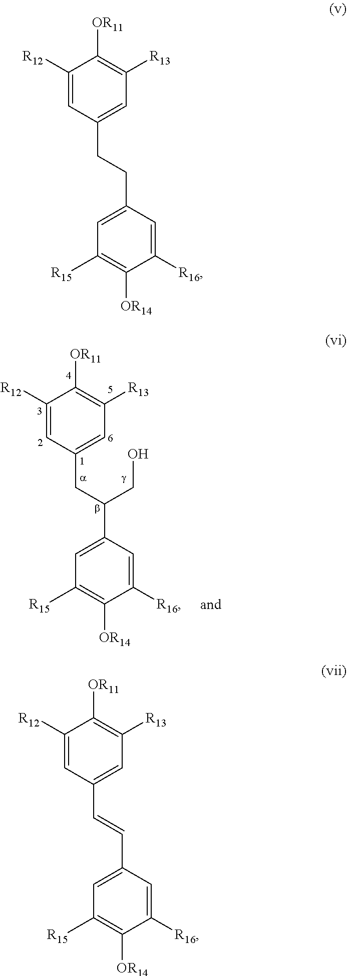
and
wherein each R₁₂, R₁₃, R₁₅ and R₁₆ is independently selected from —H, —OH, O—CH₃,
wherein R₁₁ and R₁₄ is independently selected from —H,
and/or
III) wherein at the aromatic compounds comprise at least one aromatic compound selected from the group of formula: 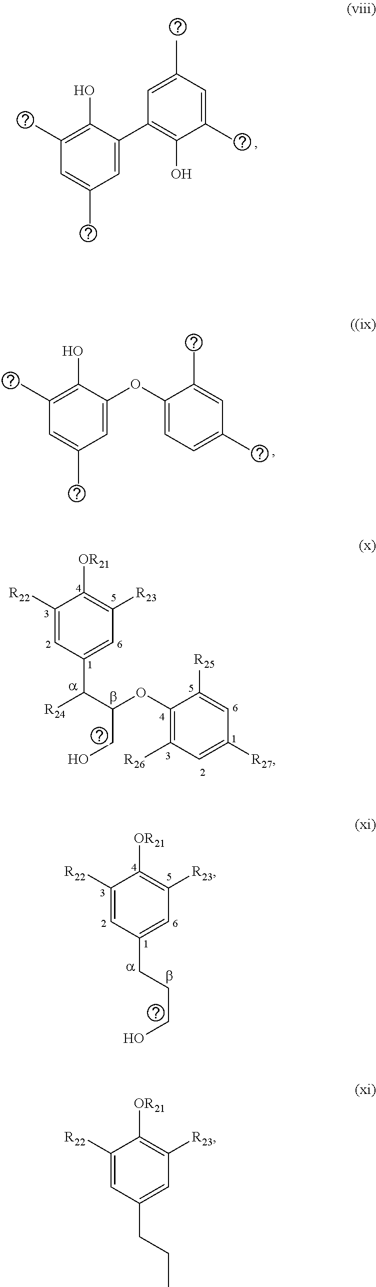 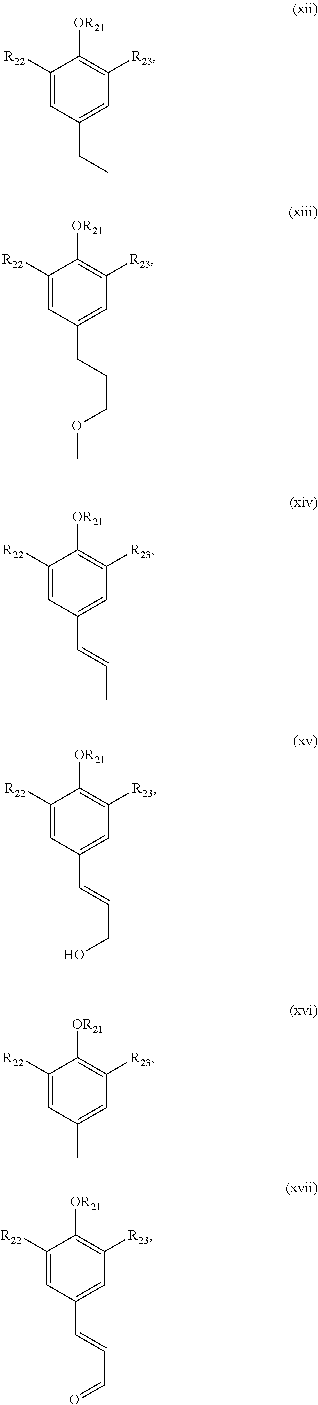 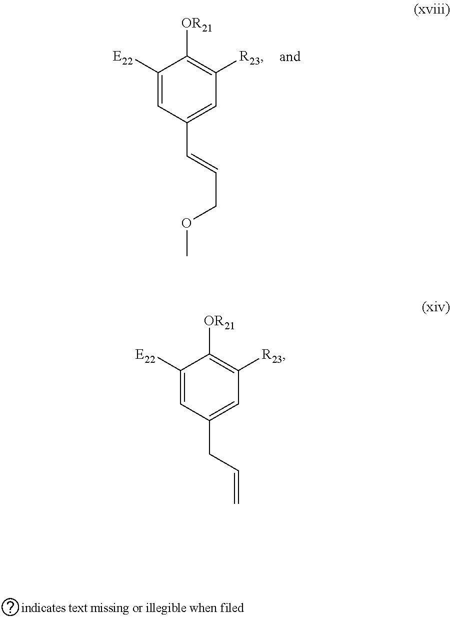
each with at least one linkage to an aromatic monomer or aromatic oligomer and
wherein R₂₁ is independently selected from —H, a β-O-4 linkage to an aromatic monomer, a 4-O-5 linkage to an aromatic monomer, an α-O-4 linkage to an aromatic monomer or a carbon-oxygen linkage to an aromatic monomer,
wherein each R₂₂ and R₂₃ is dependently selected from —H, —OH, O—CH₃ or where at least R₂₂ or R₂₃ is independently selected from a 4-O-5 linkage to an aromatic monomer, a 5-5 linkage to an aromatic monomer, a β-5 linkage to an aromatic monomer, a carbon linkage to an aromatic monomer, or a carbon-oxygen linkage to an aromatic monomer,
wherein R₂₄ is independently selected from —H, OH, or —O-alkyl wherein the alkyl group is derived from the alcohol solvent of the process,
wherein each R₂₅ and R₂₆ is independently selected from —H, —OH, O—CH₃, a 4-O-5 linkage to an aromatic monomer, a 5-5 linkage to an aromatic monomer, a β-5 linkage to an aromatic monomer, a carbon linkage to an aromatic monomer, or a carbon-oxygen linkage to an aromatic monomer,
wherein R₂₇ is independently selected from —H, end-unit selected from CH₃, —CH₂CH₃, —(CH₂)₂CH₃, —CH₂CH═CH₂, —CH═CHCH₃, —(CH₂)₂CH₂OH, —(CH₂)₂CHO, —CH═CHCH₂OH, —(CH₂)₂CH₂OCH₃, —CH═CHCH₂OCH₃, —(CH₂)₂CH₂OCH₂CH₃, —CH═CHCH₂OCH₂CH₃, —(CH₂)₂CH₂O(CH₂)₂CH₃, —CH═CHCH₂O(CH₂)₂CH₃, —(CH₂)₂CH₂OCH(CH₃)₂, —CH═CHCH₂OCH(CH₃)₂, —(CH₂)₂CH₂O(CH₂)₃CH₃, —CH═CHCH₂O(CH₂)₃CH₃.

5. The plant defense elicitor of claim 1, wherein the at least one lignin oligomer comprises at least one aromatic compound selected from the group of formulae: 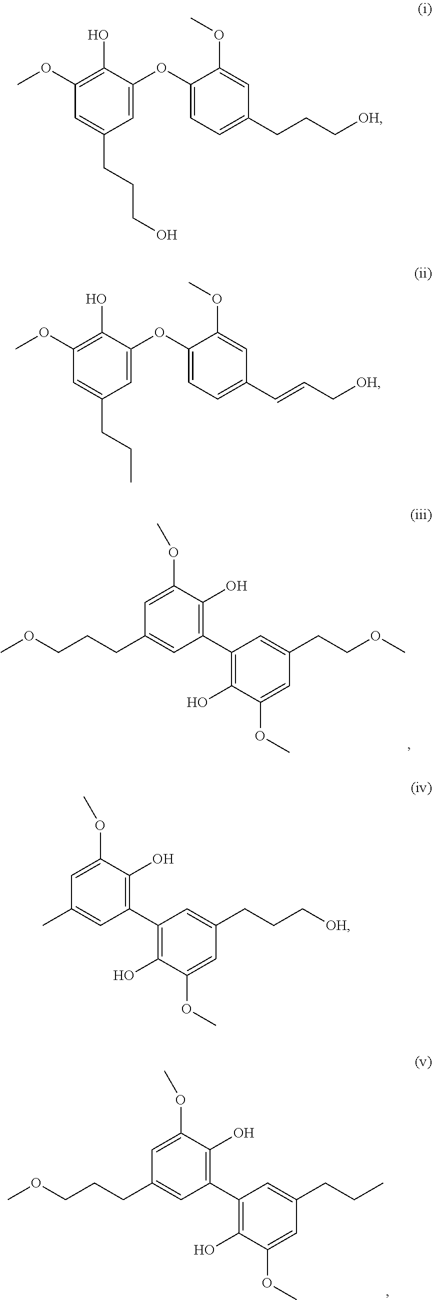 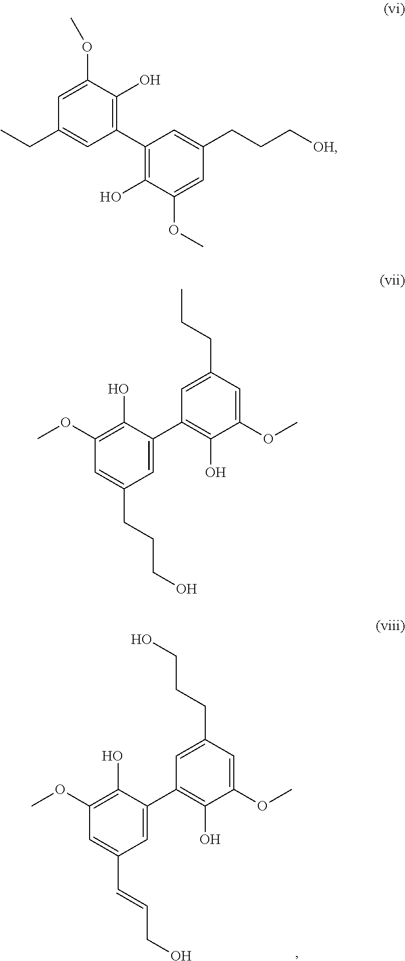
or a combination thereof.

6. The plant defense elicitor of claim 1, wherein the at least one lignin oligomer is a phenolic oligomer comprising two benzene rings directly bridged or bridged with a common bridging group selected from the group consisting of aliphatic chains, alkene groups, carbonyl groups and ether linkages or wherein the phenolic oligomer has two aromatic groups.

7. The plant defense elicitor of claim 1, wherein the at least one lignin oligomer is a phenolic oligomer comprising two benzene rings directly bridged or bridged with a common bridging group selected from the group consisting of —CH₂— groups, —CH═CH—, —C(═O)-and —O—.

8. The plant defense elicitor of claim 1, wherein the one or more lignin oligomers is present at a concentration of 50% to 90% by weight of the plant defense elicitor.

9. The plant defense elicitor of the claim 1, wherein it contains less than 0.1% each of acetic acid, methanol, and ethanol.

10. The plant defense elicitor of claim 1, comprised within a phytopharmaceutical or agrochemical composition, wherein the dry weight of the composition contains between 0.5 to 30 wt % by dry weight of lignin oligomers.

11. The plant defense elicitor of claim 1, comprised within a phytopharmaceutical or agrochemical composition, and further comprising one or more one or more anionic, non-ionic, amphoteric, or cationic surfactant, or a combination thereof.

12. The plant defense elicitor of claim 1, comprised within a phytopharmaceutical or agrochemical composition, and further comprising a lignin oligomer solubilizing agent, preferably one or more polar aprotic solvent.

13. A method for producing the phytopharmaceutical or agrochemical composition of claim 10, the method comprising:
a. subjecting lignin or lignocellulose in a liquid phase to solvolytic lignin depolymerization,
b. fractionating the extract to obtain a purified fraction enriched in lignin oligomers, and
c. formulating the purified fraction into a phytopharmaceutically or agrochemically acceptable dosage form.

14. A method of increasing abiotic and/or biotic stress tolerance in a plant, the method comprising:
contacting the plant or the plant's organ with an effective amount of the plant elicitor of claim 1.

15. A method of increasing abiotic and/or biotic stress tolerance in a plant, the method comprising:
contacting the plant or the plant's organ with an effective amount of the plant elicitor of claim 4.

16. The method according to claim 15, wherein lignin oligomers comprise:
I) at least one aromatic compound selected from the formulae: 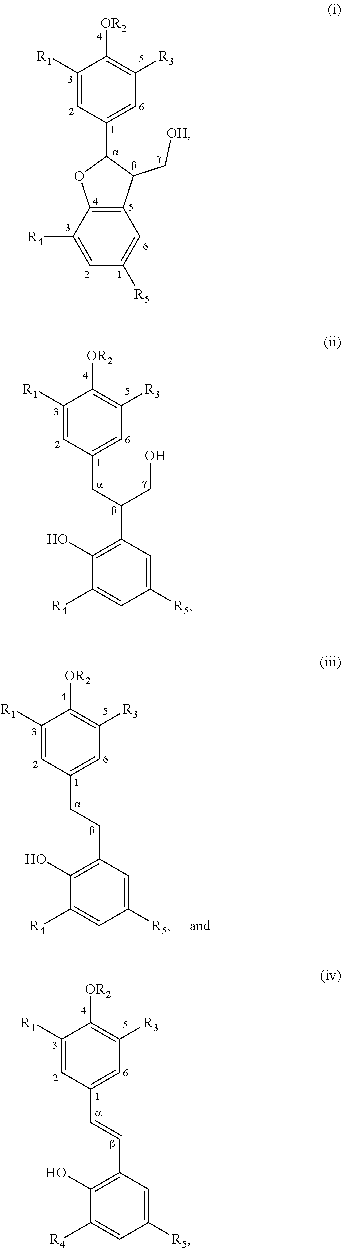
and
wherein each R₁, R₃, and R₄ is independently selected from —H, —OH, O—CH₃,
wherein R₂ is —H,
wherein R₅ is selected from —H, an end-unit selected from CH₃, —CH₂CH₃, —(CH₂)₂CH₃, —CH₂CH═CH₂, —CH═CHCH₃, —(CH₂)₂CH₂OH, —(CH₂)₂CHO, —CH═CHCH₂OH, —(CH₂)₂CH₂OCH₃, —CH═CHCH₂OCH₃, —(CH₂)₂CH₂OCH₂CH₃, —CH═CHCH₂OCH₂CH₃, —(CH₂)₂CH₂O(CH₂)₂CH₃, —CH═CHCH₂O(CH₂)₂CH₃, —(CH₂)₂CH₂OCH(CH₃)₂, —CH═CHCH₂OCH(CH₃)₂, —(CH₂)₂CH₂O(CH₂)₃CH₃, —CH═CHCH₂O(CH₂)₃CH₃;
II) wherein the aromatic compounds comprise at least one aromatic compound selected from the formulae: 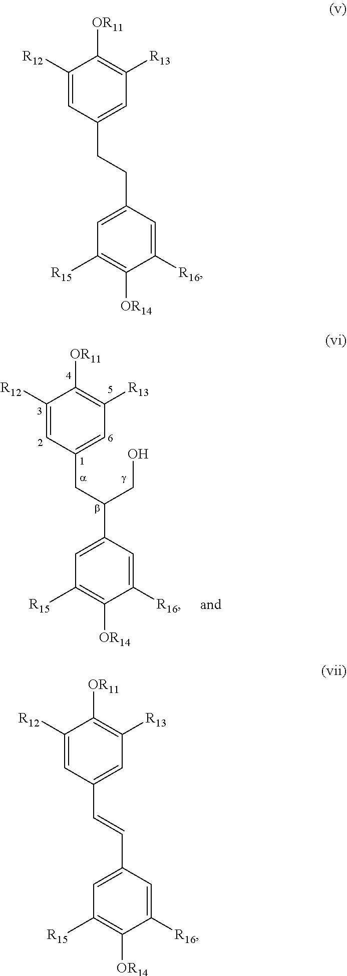
and
wherein each R₁₂, R₁₃, R₁₅ and R₁₆ is independently selected from —H, —OH, O—CH₃,
wherein R₁₁ and R₁₄ is independently selected from —H;
and/or
III) wherein at the aromatic compounds comprise at least one aromatic compound selected from the group of formula: 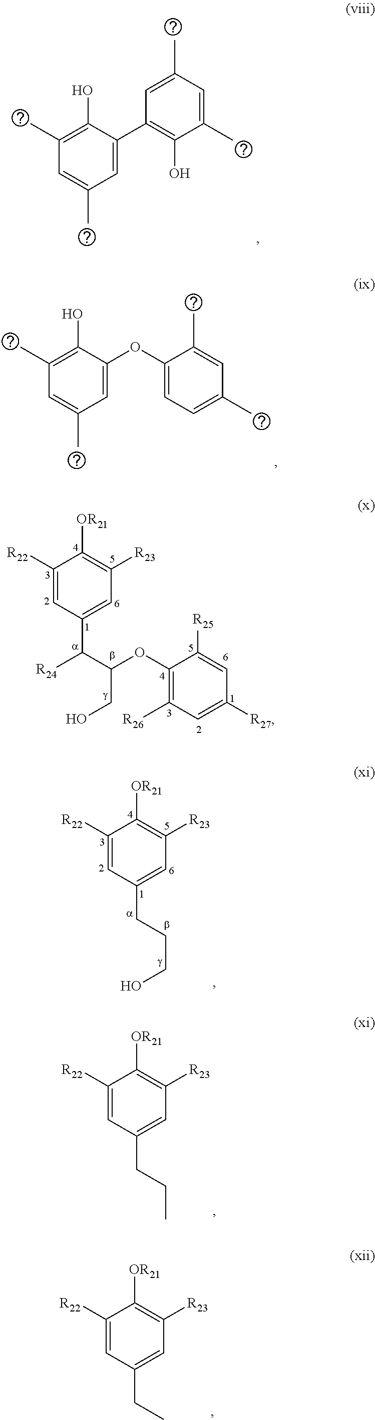 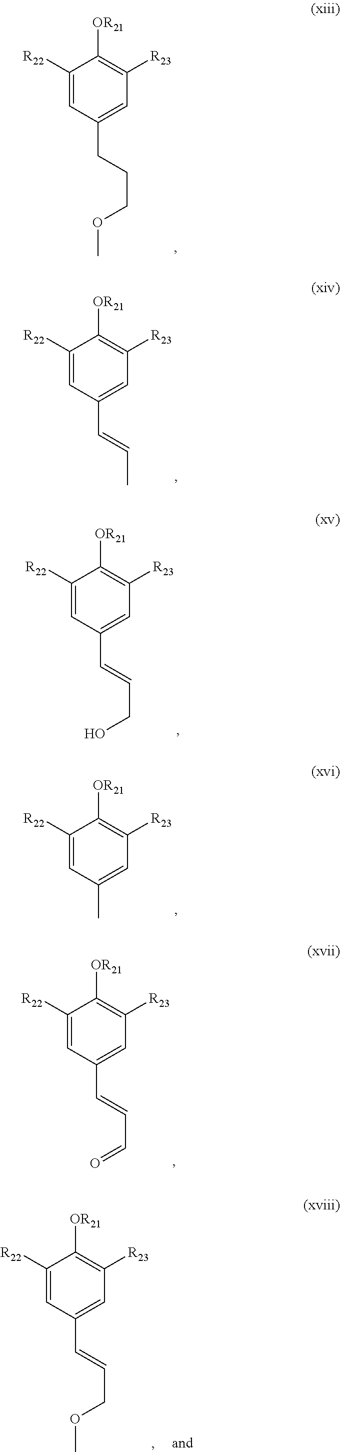 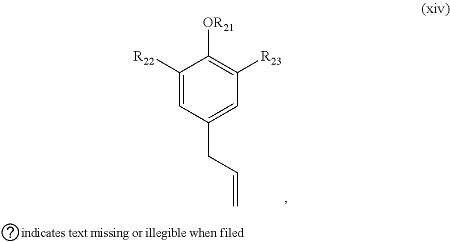
each with at least one linkage to an aromatic monomer or aromatic oligomer and
wherein R₂₁ is independently selected from —H, a β-O-4 linkage to an aromatic monomer, a 4-O-5 linkage to an aromatic monomer, an α-O-4 linkage to an aromatic monomer or a carbon-oxygen linkage to an aromatic monomer,
wherein each R₂₂ and R₂₃ is dependently selected from —H, —OH, O—CH₃ or where at least R22 or R23 is independently selected from a 4-O-5 linkage to an aromatic monomer, a 5-5 linkage to an aromatic monomer, a β-5 linkage to an aromatic monomer, a carbon linkage to an aromatic monomer, or a carbon-oxygen linkage to an aromatic monomer,
wherein R₂₄ is independently selected from —H, OH, or —O-alkyl wherein the alkyl group is derived from the alcohol solvent of the process,
wherein each R₂₅ and R₂₆ is independently selected from —H, —OH, O—CH₃, a 4-O-5 linkage to an aromatic monomer, a 5-5 linkage to an aromatic monomer, a β-5 linkage to an aromatic monomer, a carbon linkage to an aromatic monomer, or a carbon-oxygen linkage to an aromatic monomer,
wherein R₂₇ is independently selected from —H, end-unit selected from CH₃, —CH₂CH₃, —(CH₂)₂CH₃, —CH₂CH═CH₂, —CH═CHCH₃, —(CH₂)₂CH₂OH, —(CH₂)₂CHO, —CH═CHCH₂OH, —(CH₂)₂CH₂OCH₃, —CH═CHCH₂OCH₃, —(CH₂)₂CH₂OCH₂CH₃, —CH═CHCH₂OCH₂CH₃, —(CH₂)₂CH₂O(CH₂)₂CH₃, —CH═CHCH₂O(CH₂)₂CH₃, —(CH₂)₂CH₂OCH(CH₃)₂, —CH═CHCH₂OCH(CH₃)₂, —(CH₂)₂CH₂O(CH₂)₃CH₃, and —CH═CHCH₂O(CH₂)₃CH₃.

17. The method according to claim 14, wherein the composition contains less than 0.1%, each of acetic acid, methanol, and ethanol.

18. The method according to claim 14, wherein in the lignin oligomers are comprised within a composition with a pH in the range of 4 to 10.

19. The method according to claim 14, wherein the lignin oligomers are comprised within a composition that further comprises at least one anionic, non-ionic, amphoteric, or cationic surfactant, or a combination thereof, and at least one polar aprotic solvent.

20. The method according to claim 14, which induces systemic resistance in a plant against a pathogen stressor or against an abiotic stressor.

21. The method according to claim 20, wherein the plant stressor is a pathogen selected from the group consisting of fungi, bacteria, viruses, viroids, mycoplasma-like organisms, protozoa, insects, acari, and nematodes.

22. The method according to claim 20, wherein the plant stressor is an abiotic stress selected from the group consisting of hydric stress, drought and osmotic stress.

23. The method according to claim 1, comprising spraying, drenching, soaking, dipping, injection, and any combination thereof of the plant or a plant's organ with the composition.
